(19) United States
(12) Patent Application Publication
(10) Pub. No.: US 2024/0399118 A1
(43) Pub. Date: Dec. 5, 2024

(54) INTRALUMINAL LOCAL DRUG DELIVERY

(71) Applicant: Isola Therapeutics, Inc., Minneapolis, MN (US)

(72) Inventors: Brian H. CRAIG, Minneapolis, MN (US); Dwight MEGLAN, Westwood, MA (US)

(21) Appl. No.: 18/680,666

(22) Filed: May 31, 2024

Related U.S. Application Data
(60) Provisional application No. 63/505,256, filed on May 31, 2023.
(60) Provisional application No. 63/513,632, filed on Jul. 14, 2023.

(51) Int. Cl. A61M 25/10; A61B 18/00

(52) U.S. Cl. CPC A61M 25/1011; A61B 2018/0022; A61B 2018/00791

(57) ABSTRACT

Devices, systems and methods for localized delivery of a chemotherapy, hormonal therapy or targeted drug/biologic therapy to a target tissue area of an internal body organ of a patient. A catheter forms a sealed treatment chamber in a natural lumen extending through the target tissue area. Air is purged from the chamber, which is then filled with a liquid drug solution for an adequate treatment session time, solution volume and drug concentration to saturate the target tissue area, thereby providing the treatment. The drug concentration in the liquid soaking solution may be formulated to approximate the known maximum plasma concentration achieved during systemic delivery of the same drug. An integrated pressure relief circuit may be included to avoid unsafe pressure levels in the isolated chamber. The chamber is evacuated at the end of the treatment session.

### CROSS-REFERENCE TO RELATED APPLICATION

This application claims priority to U.S. Provisional Application No. 63/513,632, filed Jul. 14, 2023 and U.S. Provisional Patent Application No. 63/505,256 filed May 31, 2023, the entireties of which are incorporated herein by reference.

### TECHNICAL FIELD

The present disclosure relates to methods for delivery of a therapeutic solution to a target tissue area of an internal body organ of a patient and, more particularly, relates to intraluminal catheter systems and methods for treatment of cancer and other diseases by localized delivery of therapeutic solutions, including chemotherapy, hormonal therapy or targeted drug/biologic therapy. In some implementations, the intraluminal catheter systems and methods may be also be directed to performing an endobronchial lavage (EBL) procedure within the lung(s) as a medical countermeasure against inhaled chemical agents, including lung injury associated with phosgene gas exposure and chlorine gas exposure. It may also be directed to performing and EBL procedures after smoke inhalation.

### BACKGROUND

Nearly all chemotherapeutics are systemic, e.g., affecting the entire body, which creates the following limitations:
Toxicity: Systemic toxicity can create issues that result in restricting therapeutic dosing and are associated with a range of adverse effects that are either life threatening, e.g. immunosuppression, neutropenic enterocolitis, gastrointestinal distress, tumor lysis syndrome, organ damage, or are lifestyle limiting, e.g. anemia, fatigue, nausea/vomiting, hair loss, infertility, teratogenicity, peripheral neuropathy, cognitive impairment, potentially making chemotherapy dangerous or at least stressful to the body.
Repeat Dosing: Most chemotherapeutics are delivered intravenously (IV) but some can be delivered orally, which requires they be prepared in a way that allows the drug to survive stomach acid while being able to be absorbed in the intestines. Most require multiple doses, which require ongoing risk to potential adverse events and patient compliance to dosing regimens.

Additionally, a treatment for the lungs exposed to a chemical agent may be advantageous. For example, phosgene (COCl₂) is a colorless gas or liquid with an odor described like musty hay or green corn. Used as a chemical weapon during World War I, phosgene is frequently used today across many industries as a component of common chemicals, pesticides, and pharmaceutical products. It can cause damage to the skin, eyes, nose, throat, and lungs, and be fatal at high concentrations. When an accidental or intentional phosgene gas exposure or release occurs, phosgene can damage the blood-air barrier (also called the alveolar-capillary barrier or membrane) in the lungs, reducing the lungs' ability to deliver oxygen to the bloodstream and remove carbon dioxide. This can also cause fluid to accumulate in the lungs (pulmonary edema), leading to respiratory failure.

### SUMMARY

It is an object of the present disclosure to provide a lung treatment to mitigate pulmonary edema (excess fluid in the lungs) and other acute lung injuries that may occur from exposure to, and/or inhalation of, a chemical agent, for e.g., phosgene gas, chlorine, sulfur mustard, opioids, nerve agents, and/or any other inhaled chemical agent.

Disclosed are methods and apparatus for performing an endobronchial lavage (EBL) procedure within the lung(s) as a medical countermeasure against inhaled chemical agents, including lung injury associated with phosgene gas exposure. In a method for administering a therapeutic substance to a target tissue within a lung of a subject for treating a lung injury due to a chemical exposure, in an aspect of the method a step may be performed of advancing a catheter system into a trachea of the subject to place a tracheal catheter of the catheter within the trachea such that an expanded balloon of the tracheal catheter contacts and seals against a distal portion of the trachea. In an aspect of the method a step may be performed of positioning a first catheter of the catheter system, which slidingly extends through the tracheal catheter, within a first lung of the subject or patient such that an expanded balloon of the first catheter contacts and seals against a main bronchus of the first lung. In an aspect of the method a step may be performed of positioning a second catheter of the catheter system, which slidingly extends through the tracheal catheter, within a second lung of the subject or patient such that an expanded balloon of the second catheter contacts and seals against a main bronchus of the second lung. In an aspect of the method a step may be performed of forming a treatment chamber comprising the target tissue in one of the first and second lungs, wherein the treatment chamber is formed distal of the expanded balloon of one of the first and second catheters. In an aspect of the method a step may be performed of altering the temperature of the liquid lavage solution, in a container, to a treatment temperature that is either higher or lower than body temperature during a treatment session, the liquid lavage solution comprising the therapeutic substance. In an aspect of the method a step may be performed of conducting an endobronchial lavage (EBL) procedure in the lung to be treated, wherein the EBL procedure includes circulating the liquid lavage solution through the treatment chamber in the lung to be treated to continually contact the target tissue with the liquid lavage solution during the treatment session.

### BRIEF DESCRIPTION OF THE DRAWINGS

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

FIG. 1 illustrates a therapeutic treatment of a portion of an intestine using a catheter in accordance with an embodiment of the disclosure.

FIG. 2 shows a distal portion of a catheter in accordance with another embodiment of the disclosure.

FIG. 3 is a graph of an exemplary known relationship of diameter to inflation volume for a sealing balloon in accordance with the disclosure.

FIG. 4 shows several exemplary positions a patient may assume during a treatment procedure in accordance with the disclosure.

FIG. 5 shows a distal portion of a catheter in accordance with another embodiment of the disclosure.

FIG. 6 is a schematic view of a treatment system in accordance with the disclosure.

FIG. 7 illustrates a therapeutic treatment of a portion of a female genital tract using a catheter in accordance with another embodiment of the disclosure.

FIGS. 8-10 illustrate a therapeutic treatment of a portion of a respiratory tract using a catheter in accordance with another embodiment of the disclosure.

FIGS. 11 and 12 illustrate devices for membrane degasification of a liquid drug solution in accordance with another aspect of the disclosure.

FIG. 13 is a semi-schematic illustration of a treatment system including a pressure relief circuit in accordance with another embodiment of the disclosure.

FIG. 14 is an illustration of a holding container in accordance with an embodiment of the disclosure.

FIG. 15 is a schematic illustration of a treatment system in accordance with an embodiment of the disclosure.

FIG. 16 is an illustration of a treatment system in accordance with another embodiment of the disclosure.

FIGS. 17A and 17B are plots of temperature differentials of a holding container as taken in the treatment system of FIG. 16 in accordance with an embodiment of the disclosure.

FIG. 18 is a plot of fluid temperatures measured at different points in the treatment system of FIG. 16 in accordance with an embodiment of the disclosure.

FIG. 19 is a flow diagram that illustrates a method for delivery of a therapeutic solution to a target tissue area of an internal body organ in accordance with an embodiment of the disclosure.

FIG. 20 is a flow diagram that illustrates a method for delivery of a therapeutic solution to a target tissue area of an internal body organ in accordance with another embodiment of the disclosure.

FIG. 21 is a flow diagram that illustrates a method for delivery of a therapeutic solution to a target tissue area of an internal body organ in accordance with another embodiment of the disclosure.

FIG. 22A illustrates an assembled catheter system in accordance with an embodiment of the disclosure.

FIG. 22B illustrates the catheter system of FIG. 22A in a disassembled state.

FIG. 23 depicts the catheter system of FIG. 22A placed in vivo to provide an endobronchial lavage (EBL) procedure within the lungs of a subject or patient.

FIG. 24 illustrates an endobronchial lavage method in accordance with an embodiment of the disclosure.

FIG. 25 shows the concentration of diclofenac in plasma following administration in test animals.

### DETAILED DESCRIPTION

Specific embodiments of the present technology are now described with reference to the figures. The terms “distal” and “proximal” are used in the following description with respect to a position or direction relative to the treating operator. “Distal” or “distally” are a position distant from or in a direction away from the operator. “Proximal” and “proximally” are a position near or in a direction toward the operator. The term “target,” as in “target tissue, target area, target organ, or target region” is used to refer to diseased tissue of a hollow organ and/or tissue of a natural tract or lumen extending therethrough. The following terms are used regarding drug delivery parameters:
TC=Tissue Concentration.
Local=Local non-circulatory delivery route
Systemic=Systemic or circulatory delivery route.
MTC=Maximum Tissue Concentration.
MSSC=Maximum Soaking Solution Concentration.
MPC=Maximum Plasma Concentration

The following detailed description is merely exemplary in nature and is not intended to limit the scope of the present technology or the application and uses of the present technology. Platforms and methods of this disclosure may reduce the limitations of systemic drug delivery.

A highly localized method of delivering therapeutic solutions may reduce complications and increase effectiveness for inductive (curative), neoadjuvant (prior to surgery), or adjuvant (after surgery) drug treatments. Such a treatment may be localized to hollow organ or natural lumens. A selected drug can be delivered in liquid, aerosol/nebulizer, or even sprayed. In some implementations, the selected drug may be heated or cooled. The hollow organ is initially flushed or lavaged with lavage fluid (e.g., water, saline, or other fluid that may or may not contain drug) to remove or extricate any debris, gas, contaminant, or other irritant from the hollow organ. The lavage fluid is removed form the hollow organ (e.g., via a syphon or by natural expression by the hollow organ). Once the lavage fluid is removed, the hollow organ is locally bathed in the drug for an extended period, e.g., about 20 minutes, to achieve drug absorption into the targeted organ tissue. Surprisingly, after bathing the organ, systemic concentrations in the body of the selected drug remain substantially low, e.g., at or below the Food and Drug Administration's (FDA) approved systemic levels for the particular drug for a particular mammal (e.g., a human). Although the description of embodiments hereof is in the context of treatments performed within a variety of natural hollow body lumens or tracts, the present technology may also be used in any other body passageways or in extraluminal locations where it is deemed useful. Furthermore, there is no intention to be bound by any expressed or implied theory presented in the preceding technical field, background, brief summary or the following detailed description.

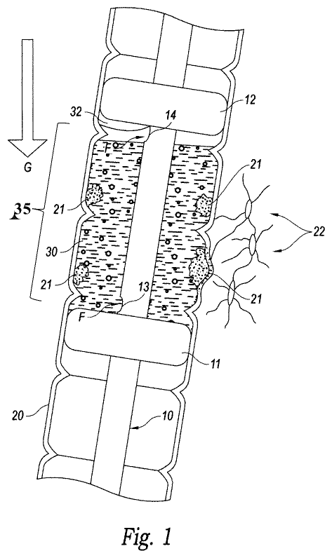

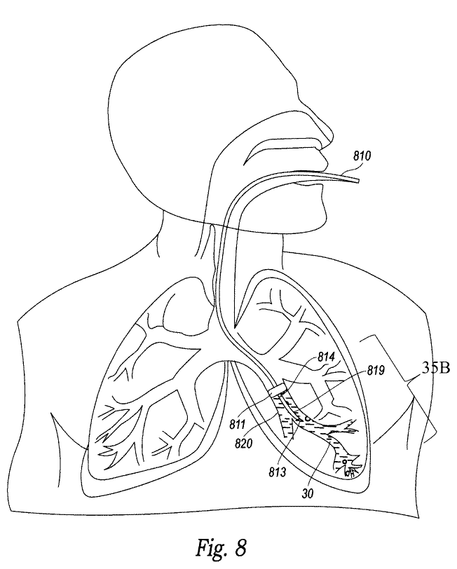

FIG. 1 illustrates a catheter 10 configured in accordance with an embodiment of the present technology. Catheter 10 having an elongate flexible shaft is shown in a deployed configuration within a target region of a natural lumen, which in this example is a portion of a large intestine or colon 20. Expandable members 11, 12 are mounted about a distal region of catheter 10, and are longitudinally spaced apart such that, when expanded into sealing contact with the inner wall of colon 20, a closed treatment chamber (e.g. treatment chamber 35) is defined between expandable members 11, 12 and the intestinal wall. According to embodiments, the treatment chamber 35 may be considered to be an annular chamber because of the annular cylinder formed between the catheter shaft and the natural lumen. Herein, “closed” means the treatment chamber 35 is excluded from fluid communication with other parts of the natural lumen beyond the expandable members. An expandable member for the present technology may be a mechanically operated sealing element or a balloon that is inflatable with a fluid that may be either a gas or a liquid. In the illustrated embodiment, the treatment chamber 35 includes one or more polyps or other cancers 21. Catheter 10 is reversible, meaning that the flexible catheter shaft may be considered to extend proximally either upward or downward in FIG. 1. Ports 13, 14 fluidly communicate the treatment chamber 35 with respective lumens (not shown) that extend proximally through the catheter to terminate at respective connecting ports 15, 16 located at the proximal end thereof, as shown in FIG. 6.

Once catheter 10 has been deployed as shown in FIG. 1, the operator may assess the orientation of the treatment chamber 35 with respect to gravity G, and may reposition the patient, if necessary, to orient the treatment chamber 35 as close to vertical as possible in order to facilitate air evacuation as the treatment chamber 35 is filled with liquid. The avoidance of air bubbles or air pockets may ensure that all of the inner wall of colon 20 in the treatment chamber 35 is bathed, or immersed, in liquid drug solution 30.

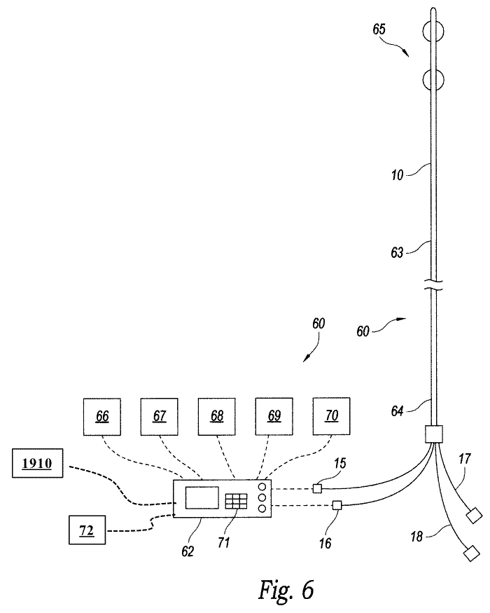

FIG. 4 shows examples of different patient positions that may provide a vertical treatment chamber. In the embodiment illustrated in FIG. 1, the portion of colon 20 enclosing the treatment chamber 35 is nearly vertical with port 14 being located at a high point in the treatment chamber 35. Because catheter 10 is reversible, the patient or the catheter could be positioned such that port 13 is located at a high point in the chamber (not shown). It is the operator's choice how to position or repositioning the patient, based on comfort of the patient and convenience of the operator and as a result, either of ports 13, 14 may become the upper port in the treatment chamber 35. The upper port may be defined as the egress port and the other, lower port may then be defined as the ingress port. Once the treatment chamber 35, catheter lumens, extension lines (if used), and pump are purged of air as described below, the patient may be repositioned or returned to a position that may be more comfortable for the patient and/or more convenient for the operator.

According to one embodiment, after the treatment chamber 35 is oriented with respect to gravity, a liquid drug solution 30 is optionally heated and admitted or pumped into the treatment chamber 35 via the ingress port, i.e., port 13 in FIG. 1. As liquid drug solution 30 fills the treatment chamber 35 from bottom to top, air is purged from the treatment chamber 35 via the egress port, i.e., port 14 until the liquid drug solution reaches port 14. See flow arrows F in FIG. 1. Optionally, a partial vacuum may be applied to egress port 14 to assist or hasten the purging process. In this way, the treatment chamber 35 is filled with liquid drug solution 30, leaving only a small air bubble 32, or preferably no air bubble at all. After the treatment chamber 35 is filled with liquid drug solution 30, air may also be purged from all catheter lumens, extension lines (if used), and a circulating pump, such as pump 67 described below, to form a closed fluid circuit that may be a closed-loop fluid circuit. Optionally, as described in further detail below, liquid drug solution 30 is heated to a treatment temperature prior to delivery, and liquid drug solution 30 is maintained at the treatment temperature during a treatment session as it is circulated throughout the closed fluid circuit.

In an alternative purging method, a supply reservoir, catheter lumens, treatment chamber and a waste container forms a closed fluid circuit. A pressure difference between the elevated supply reservoir and the lower treatment chamber induces a flow through the fluid circuit (e.g., gravity feed without a pump). For example, after the treatment chamber 35 is oriented with respect to gravity, a lavage liquid, such as sterile saline (e.g. lavage fluid), is pushed into the chamber via the ingress port, i.e., port 13 in FIG. 1. As the lavage fluid fills the treatment chamber 35 from bottom to top, air is purged from the chamber via the egress port, i.e., port 14 until the lavage fluid reaches port 14. See flow arrows F in FIG. 1. After the treatment chamber 35 is filled with lavage fluid, air may also be purged from all catheter lumens, extension lines (if used). The lavage fluid in the fluid circuit removes any debris, gas, contaminant, or other irritant from the hollow organ and the saline can then be replaced with liquid drug solution 30. As described herein, a therapeutic substance includes liquid drug solution 30, saline, or any other fluid used for therapeutic value. As described in further detail below, in some implementations, a therapeutic substance can be heated to a treatment temperature prior to or during delivery to the treatment chamber 35. In another instance, the therapeutic substance may further be maintained at the treatment temperature during a treatment session as the therapeutic substance is circulated throughout the closed fluid circuit. In some implementations, the therapeutic substance is not heated, e.g., at room temperature (about 18-24° C.) or below room temperature (about 5-18° C.).

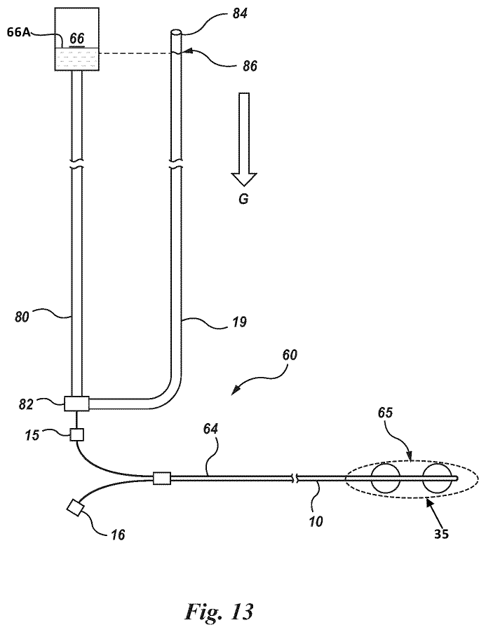

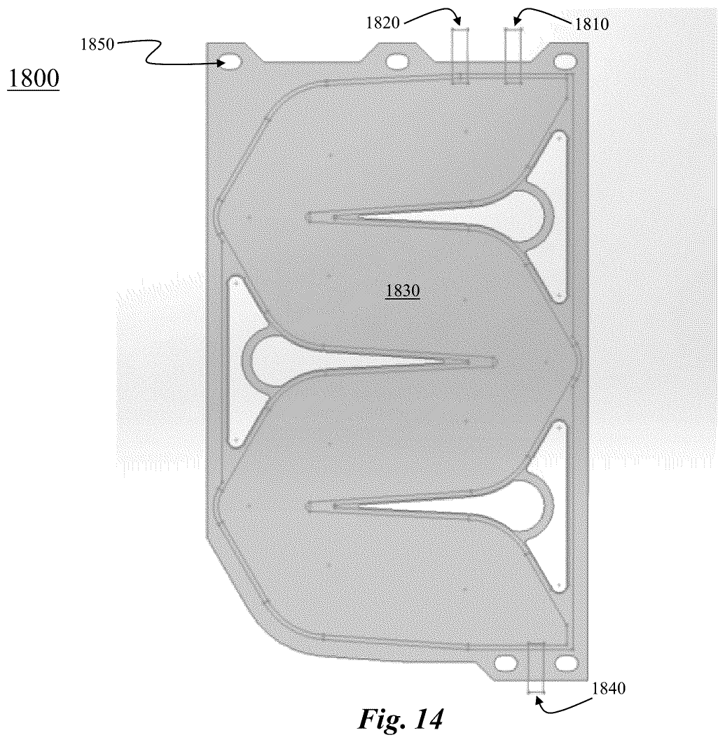

Once the closed fluid circuit is purged of air and contaminants and filled with liquid drug solution 30, a treatment session may then be conducted by circulating the liquid drug through the closed fluid circuit to maintain a homogeneous concentration of the drug throughout the treatment chamber 35. For example, the concentration of the liquid drug solution 30 may be much higher than the FDA's approved systemic concentration for the drug, e.g., ten times (10X) an FDA approved systemic concentration. Herein, “circulating” means causing a fixed volume of liquid drug solution 30 to flow through the closed fluid circuit. According to an embodiment, “circulating” includes causing a fixed volume of liquid drug solution 30 to flow through the closed fluid circuit between first and second external reservoirs (e.g., first and second syringes or containers/bags connected to respective ports 15, 16 shown in FIG. 6 and/or a container elevated about the treatment chamber 35 and connected to the catheter 10 as shown in FIG. 13) or a single external reservoir (e.g., holding container 1800 shown in FIG. 14) without intentional loss of the liquid solution either inside or outside of the patient. The circulating flow of liquid drug solution 30 may be unidirectional during the treatment session or may reverse direction one or more times between the first and second reservoirs. Pushing or filling a liquid, cither a drug solution or saline, from a first graduated syringe through the closed fluid circuit and into a second graduated syringe allows the operator to initially confirm and subsequently monitor seal integrity of the treatment chamber 35 by comparing input and output volumes. In some implementations, the drug solution 30 may be gravity fed through the closed fluid circuit. That is, the saline and/or liquid drug solution 30 may be supplied by a reservoir (e.g. a bag) and fed through the catheter 10 to the treatment chamber 35 via gravity. That is, the pressure due to the height of the drug container causes the drug solution to fill the interstitial space of the treatment chamber 35 until the treatment chamber is filled with drug solution.

Alternatively, connecting ports 15, 16 may be connected to input and output ports of a pump (e.g., pump 67, shown in FIGS. 6, 15 and 16) thereby forming a closed-loop fluid circuit. Herein, a “closed-loop fluid circuit” is considered to be a subset of closed fluid circuits. In this arrangement, a treatment session may be conducted by recirculating the liquid drug solution 30 through the closed-loop fluid circuit to maintain a homogeneous concentration of the drug throughout the treatment chamber 35. Herein, “recirculating” means causing liquid drug solution 30 to continuously flow (e.g., via pump 67) through a closed-loop fluid circuit without intentional loss of the liquid solution either inside or outside of the patient. As used herein, “circulating” and “recirculating” are used interchangeably.

To conduct a treatment session safely and effectively in accordance with an embodiment of the present technology, it may be useful to predetermine a desired dose of drug to permeate or be dispensed or absorbed into the target tissue, and to measure, monitor, calculate or otherwise estimate attainment or progress towards that pharmacokinetic goal during or at the end of a treatment session. Surprisingly, due to the drug solution being in liquid form, the tissues of the organ may be treated without the hollow organ absorbing an amount of the drug that exceeds the FDA's approved systemic levels for the particular drug. Additionally, as discussed in greater detail below, certain embodiments heat the administered drug to a treatment temperature prior to and/or during delivery of the drug to further improve the safety and effectiveness of the treatment session. However, testing has shown that treating a hollow organ with heated or unheated drug solution does not impact tolerance of the treatment by the patient.

To predetermine the desired dose, it may be useful to estimate the volume of tissue targeted for saturation with the drug molecules from drug solution 30. Target tissue volume may be estimated based on the surface area of the tissue comprising the treatment chamber 35 in a given patient. To predetermine the desired dose, it may also be useful to know or estimate the rate of transfer of the drug (heated or otherwise) through the wall of the natural lumen and into the target tissue area.

One parameter that may be used to calculate the exposed tissue surface area may be the liquid capacity of the treatment chamber 35 as measured by the volume of liquid pumped into the fluid circuit during the air purging step. For example, drug solution 30 or sterile saline may be admitted by a graduated syringe to the ingress port via one of connecting ports 15 or 16 shown in FIG. 6, and the volume of admitted liquid drug solution 30 is measured when the liquid begins to appear at the other of connecting ports 15 or 16 in fluid communication with the purge port.

Other parameters that may be used to calculate the exposed tissue surface area may be a known distance between the pair of expandable members, a diameter of at least one of the expandable members, a distance from the natural orifice of the natural lumen to the two or more expandable members, an analysis of current and/or previous medical images of the natural lumen extending through the target tissue area of the internal body organ of the patient, and a statistical analysis of historical data regarding physical dimensions of similar natural lumens extending through similar target tissue areas for a known population of patients. The diameter of at least one of the expandable members may be measured from a medical image or the expandable member may be an inflatable elastic balloon and a diameter of the balloon is determined based at least in part on a volume of a fluid or air used to inflate the balloon into sealing contact with the inner wall of the natural lumen.

A treatment session may be terminated when the desired drug dose has been delivered to the target tissue. The amount of drug delivered via the treatment chamber 35 may be estimated using parameters including the volume of the closed-loop fluid circuit, the volume of the target tissue, and the change in concentration of the drug in recirculating liquid drug solution 30. Thus, the amount of drug calculated as missing from the volume of liquid in the closed-loop fluid circuit is presumed to have permeated into the target tissue.

An alternative method of estimating the amount of drug delivered during a treatment session may be based on elapsed time and parameters such as a known permeability rate for a given concentration of drug in a given tissue type. Such parameters may be drawn from data regarding a general population rather than requiring data from the current patient. In this method, the size of the surface area of target tissue may or may not be useful to determine whether the desired drug dose has been delivered to the target tissue.

Another method in accordance with an embodiment of the present technology may continue recirculating liquid drug solution 30 (heated or otherwise) through the closed-loop fluid circuit beyond the point of saturating target tissue with a selected anti-cancer drug. The drug may permeate the target tissue, enter and activate the lymphatic system 22 or interstitial space, all of which may act as a conduit or reservoir for the drug to continue eluting drug into cancerous tissue after the session has been terminated and the catheter is removed from the patient.

Another method in accordance with an embodiment of the present technology is to fill the treatment chamber 35 with liquid drug solution 30 (heated or otherwise) of a known, e.g., calculated drug concentration for a selected period of time without circulation or recirculation. That is, liquid drug solution 30 carries a measured amount of the drug and remains stationary in the treatment chamber 35 for a duration that is expected to achieve the desired drug dosing.

Another method in accordance with an embodiment of the present technology is to heat liquid drug solution 30 to a treatment temperature (e.g., between 37° C. and 44° C., such as 42° C.) prior to circulation or recirculation. Yet another method in accordance with an embodiment of the present technology is to heat liquid drug solution 30 to a treatment temperature prior to circulation or recirculation and then maintain the liquid drug solution 30 at the treatment temperature during the treatment session (e.g., during circulation or recirculation of the drug solution 30). That is, according to certain embodiments herein, when liquid drug solution 30 is raised to a treatment temperature, the liquid drug solution 30 that has been raised to the treatment temperature surprisingly encourages better tissue uptake of the drug over conventional therapies. For certain applications, such as chemotherapy, pneumonia treatment, etc., raising the liquid drug solution 30 to treatment temperature increases the apoptosis of cancer cells (or generally reduces and/or eliminates the pathogen-such as a bacterial or viral infection-causing a disease) when compared to the same dosage at room temperature.

Alternatively, a treatment session may be terminated when an amount of drug (heated or otherwise) measured in the patient's blood reaches a predetermined level, which may be selected to be a level indicating that the desired drug dosage has been delivered to the target tissue. A predetermined threshold of drug concentration in the blood may also be set such that drug concentration in the blood above that level may be considered to be approaching a toxic condition. The amount of drug detected in the patient's bloodstream may indicate that the selected anti-cancer drug has been absorbed from the non-vascular natural hollow body lumen, has saturated the target tissue, and has begun entering the vasculature.

Measuring drug concentration in a patient's blood during treatment may be a particularly sensitive and useful monitoring technique in treatments where the target tissue is highly vascularized, for example in the lungs. Monitoring of a patient's blood plasma or serum drug level may be done by intermittent blood sampling, e.g., via a venipuncture or an indwelling arterial or central venous line. Alternatively, blood plasma or serum drug level may be monitored continuously in real time by circulating the patient's blood through a measuring device such as console 62 below, and associated components similar to pump 67 and osmometer 68. In such an arrangement, console 62 can notify a clinician and/or terminate treatment if an amount of drug measured in the patient's blood reaches a predetermined level. Similarly, measuring the temperature of liquid drug solution 30 is used in certain embodiments described below and may be done with temperature gauges (e.g., thermometers) taking measurements at different places of the treatment system. Such embodiments are beneficial where the target tissue is highly vascularized, for example in the lungs.

In previous animal studies, applicant discovered that for chemotherapy via a non-circulatory route as disclosed herein, tissue dosing can be predicted using known pharmacokinetic data for systemic chemotherapy. Systemic delivery achieves tissue dosing by saturating organ tissue via the circulatory system. Maximum organ tissue concentration (MTC) is directly correlated to maximum plasma concentration (MPC) in systemic delivery but varies by drug according to each drug's unique pharmacokinetics.

It was concluded that the absorption of drug by organ tissue is equivalent between systemic and non-systemic routes of delivery. Thus, similar lung tissue concentration TC can be achieved regardless of method to saturate lung tissue, i.e., systemic delivery via the circulatory system or local delivery directly to the lung tissue, as long as the MPC and MSSC are similar and the dwell time for the selected method is sufficient to ensure tissue saturation. This observation allows data from systemic drug delivery (oral, IV, etc.) to be used to set soaking solution concentration and predict lung tissue dosing via local drug delivery (endoluminal, ultrasound, needle, etc.). The reverse is also possible: Data from non-systemic local drug administration can be used for systemic therapy to set plasma drug concentration targets and predict resulting tissue dosing.

$$T {C}_{L ocal} ≅ {TC}_{Systemic} when {MSSC}_{Systemic} ≅ {MSSC}_{Local} @ tissue saturation or when {MPC}_{Systemic} ≅ {MSSC}_{L ocal} @ tissue saturation$$

If tissue saturation is not achieved with local delivery due to insufficient penetration of drug, e.g., insufficient drug dwell time, target tissue too far from drug delivery site, etc., then it can be assumed that the tissue concentration will be less than the projected value.

As shown in the examples below, local delivery of drugs that did not result in significant systemic effects was achieved in animal experiments with diclofenac, sodium bicarbonate and dexamethasone sodium phosphate indicating that this methodology may work for any pharmaceutical. Additionally, it is expected that this experience in the lung is applicable to any bodily organ, including but not limited to colon, rectum, stomach, esophagus, bladder, liver, kidney, and pancreas.

The ability to directly target a diseased organ in a subject with local administration of a therapeutic solution (heated or otherwise) provides several advantages over traditional systemic treatment methods. In embodiments where the drug is a chemotherapeutic agent, local administration of the therapeutic solution (heated or otherwise) provides the advantage of delivering the drug directly to the organ where it is needed, as opposed to circulating the drug throughout the body as is done with systemic administration. This allows the chemotherapeutic agent-which is highly efficient at preventing cell growth and/or killing cells—to be directly applied to the cancerous tissues without attacking normal tissues in other parts of the body. In embodiments, an advantage to targeted local administration of the therapeutic solution (heated or otherwise) is the reduction or near elimination of side effects of the chemotherapeutic or other therapeutic agent. In embodiments, these side effects include one or more of nausea, vomiting, diarrhea, hair loss, loss of appetite, fatigue, fever, mouth sores, pain, constipation, easy bruising or bleeding. In embodiments, these side effects of chemotherapeutic agents may require the subject to shorten their course of chemotherapy or may cause the subject to be contraindicated from administration of chemotherapy all together.

The terms “drug” and “agent” are used interchangeably, and “chemotherapeutic drug” and “chemotherapeutic agent” are used interchangeably.

In embodiments, an advantage to targeted local administration of a therapeutic solution (heated or otherwise) is the ability to achieve effective results with a lower dose of drug than is required to achieve effective results with systemic delivery. As a systemically administered drug spreads throughout the body via the bloodstream and may be removed from the bloodstream when filtered through the kidneys and liver, larger doses are required in order to deliver the needed amount of drug to a specific organ. An “effective dose,” as used herein, means the dose required to achieve an improvement in, remission or elimination of the disease state. In embodiments where the drug is a chemotherapeutic agent, an “effective dose,” as used herein, means the dose required to achieve a reduction in growth rate of cancer cells, a reduction in the total number of cancer cells present in the organ, or an elimination of all of the cancer cells in the organ.

In embodiments, the therapeutic agent is dosed at a concentration that allows for a “reservoir effect” where the therapeutic agent is maintained in the tissue for a period of time after contact of the therapeutic agent is discontinued. In embodiments, the therapeutic agent is dosed at a higher concentration than may otherwise be used in order to achieve a reservoir effect. In some embodiments, it may be particularly desirable to achieve a reservoir effect when treating disorders such as inflammation, pain, lung injury, lung damage, chemical poisoning and smoke inhalation.

In embodiments, local administration can achieve an effective dose at a concentration that is about 5 to about 100 fold lower than the effective dose concentration for systemic administration. In embodiments, local administration can achieve an effective dose at a concentration that is about 10 to about 50 fold lower than the effective dose concentration for systemic administration. In embodiments, local administration can achieve an effective dose at a concentration that is about 20 to about 40 fold lower than the effective dose concentration for systemic administration. In embodiments, local administration can achieve an effective dose at a concentration that is about 30 to about 40 fold lower than the effective dose concentration for systemic administration. In embodiments, local administration can achieve an effective dose at a concentration that is about 33 to about 38 fold lower than the effective dose concentration for systemic administration. In embodiments, local administration can achieve an effective dose at a concentration that is about 36 fold lower than the effective dose concentration for systemic administration. In embodiments, local administration can achieve an effective dose at a concentration that is about 37 fold lower than the effective dose concentration for systemic administration. In embodiments, local administration can achieve an effective dose at a concentration that is about 5, 10, 15, 20, 25, 30, 35, 40, 45, 50, 55, 60, 65, 70, 75, 80, 85, 90, 95 or 100 fold lower than the effective dose concentration for systemic administration. In embodiments, administering a lower effective dose leads to reduced side effects of the drug.

In embodiments, an advantage to targeted local administration is the ability to achieve effective results in a shorter treatment time. In embodiments, the local administration treatment time is for about 3, 5, 7, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 minutes. In embodiments, the local administration treatment time is for about 10, 20, 30, 40, 50, 60, 70, 80, 90, 100, 110 or 120 minutes. In embodiments, the local administration treatment time is for about 20 minutes. In embodiments, the local administration treatment time is shorter than the administration time for systemic delivery, which can be 1, 2, 3, 4, 5 hours or longer. In embodiments, the shorter treatment time leads to greater patient comfort and greater patient compliance.

In embodiments, the disclosure provides a method for treating lung cancer in a subject, including administering a liquid formulation having a therapeutic agent directly to a lung tissue in the subject. In embodiments, the methods comprise directly administering the therapeutic agent using a catheter as described herein. In embodiments, the methods include administering the liquid formulation by filling all or part of the lung with the liquid formulation.

In embodiments, the disclosure provides a method for treating lung cancer in a subject, including administering a liquid formulation having a therapeutic agent directly to a lung tissue in the subject; wherein the concentration of chemotherapeutic agent in the liquid formulation is from about 10000 ng/ml to about 500000 ng/mL. In embodiments of the method, the concentration of chemotherapeutic agent in the liquid formulation is from about 20000 ng/ml to about 200000 ng/mL. In embodiments of the method, the concentration of chemotherapeutic agent in the liquid formulation is from about 25000 ng/ml to about 100000 ng/ml. In embodiments of the method, the concentration of chemotherapeutic agent in the liquid formulation is from about 30000 ng/ml to about 50000 ng/mL. In embodiments of the method, the concentration of chemotherapeutic agent in the liquid formulation is from about 2000 ng/mL to about 20000 ng/mL. In embodiments of the method, the concentration of chemotherapeutic agent in the liquid formulation is from about 2000 ng/ml to about 15000 ng/ml. In embodiments of the method, the concentration of chemotherapeutic agent in the liquid formulation is from about 2000 ng/ml to about 10000 ng/mL. In embodiments of the method, the concentration of chemotherapeutic agent in the liquid formulation is about 1000, 1500, 2000, 2500, 3000, 4000, 5000, 7500 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000 or 50000 ng/ml. In embodiments of the method, the concentration of chemotherapeutic agent in the liquid formulation is about 30000 ng/mL. In embodiments of the method, the concentration of chemotherapeutic agent in the liquid formulation is about 31000 ng/ml. In embodiments of the method, the concentration of chemotherapeutic agent in the liquid formulation is about 32000 ng/ml. In embodiments of the method, the concentration of chemotherapeutic agent in the liquid formulation is about 33000 ng/ml. In embodiments of the method, the concentration of chemotherapeutic agent in the liquid formulation is about 34000 ng/ml. In embodiments of the method, the concentration of chemotherapeutic agent in the liquid formulation is about 35000 ng/mL.

In embodiments, the disclosure provides a liquid formulation having a chemotherapeutic agent for use in treating lung cancer in a subject, wherein the concentration of chemotherapeutic agent in the liquid formulation is from about 10000 ng/mL to about 500000 ng/ml. In embodiments of the liquid formulation, the concentration of chemotherapeutic agent in the liquid formulation is from about 20000 ng/ml to about 200000 ng/mL. In embodiments of the liquid formulation, the concentration of chemotherapeutic agent in the liquid formulation is from about 25000 ng/mL to about 100000 ng/mL. In embodiments of the liquid formulation, the concentration of chemotherapeutic agent in the liquid formulation is from about 30000 ng/ml to about 50000 ng/mL. In embodiments of the liquid formulation, the concentration of chemotherapeutic agent in the liquid formulation is from about 2000 ng/mL to about 20000 ng/ml. In embodiments of the liquid formulation, the concentration of chemotherapeutic agent in the liquid formulation is from about 2000 ng/ml to about 15000 ng/ml. In embodiments of the liquid formulation, the concentration of chemotherapeutic agent in the liquid formulation is from about 2000 ng/ml to about 10000 ng/mL. In embodiments of the liquid formulation, the concentration of chemotherapeutic agent in the liquid formulation is about 1000, 1500, 2000, 2500, 3000, 4000, 5000, 7500 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000 or 50000 ng/mL. In embodiments of the liquid formulation, the concentration of chemotherapeutic agent in the liquid formulation is about 30000 ng/mL. In embodiments of the liquid formulation, the concentration of chemotherapeutic agent in the liquid formulation is about 31000 ng/mL. In embodiments of the liquid formulation, the concentration of chemotherapeutic agent in the liquid formulation is about 32000 ng/ml. In embodiments of the liquid formulation, the concentration of chemotherapeutic agent in the liquid formulation is about 33000 ng/ml. In embodiments of the liquid formulation, the concentration of chemotherapeutic agent in the liquid formulation is about 34000 ng/mL. In embodiments of the liquid formulation, the concentration of chemotherapeutic agent in the liquid formulation is about 35000 ng/mL.

In embodiments, the disclosure provides a method for treating lung damage or injury in a subject, including administering a liquid formulation having a anti-inflammatory agent directly to a lung tissue in the subject; wherein the concentration of the anti-inflammatory agent in the liquid formulation is from about 1 mcg/mL to about 100 mcg/mL. In embodiments of the method, the concentration of the anti-inflammatory agent in the liquid formulation is from about 5 mcg/mL to about 90 mcg/mL. In embodiments of the method, the concentration of the anti-inflammatory agent in the liquid formulation is from about 10 mcg/mL to about 80 mcg/mL. In embodiments of the method, the concentration of the anti-inflammatory agent in the liquid formulation is from about 25 mcg/mL to about 75 mcg/mL. In embodiments of the method, the concentration of the anti-inflammatory agent in the liquid formulation is from about 30 mcg/mL to about 70 mcg/mL. In embodiments of the method, the concentration of the anti-inflammatory agent in the liquid formulation is from about 40 mcg/mL to about 60 mcg/mL. In embodiments of the method, the concentration of the anti-inflammatory agent in the liquid formulation is from about 45 mcg/mL to about 55 mcg/mL. In embodiments of the method, the concentration of the anti-inflammatory agent in the liquid formulation is about 1, 5, 10, 15, 20, 25, 30, 35, 40, 45, 50, 55, 60, 65, 70, 75, 80, 85, 90, 95 or 100 mcg/mL. In embodiments of the method, the concentration of the anti-inflammatory agent in the liquid formulation is about 40 mcg/mL. In embodiments of the method, the concentration of the anti-inflammatory agent in the liquid formulation is about 45 mcg/mL. In embodiments of the method, the concentration of the anti-inflammatory agent in the liquid formulation is about 50 mcg/mL. In embodiments of the method, the concentration of the anti-inflammatory agent in the liquid formulation is about 55 mcg/mL. In embodiments of the method, the concentration of the anti-inflammatory agent in the liquid formulation is about 65 mcg/mL. Example anti-inflammatory agents are provided herein.

In embodiments, the disclosure provides a method for treating lung inflammation in a subject, including administering a liquid formulation comprising diclofenac directly to a lung tissue in the subject; wherein the concentration of diclofenac in the liquid formulation is from about 1 mcg/mL to about 100 mcg/mL. In embodiments of the method, the concentration of diclofenac in the liquid formulation is from about 5 mcg/mL to about 90 mcg/mL. In embodiments of the method, the concentration of diclofenac in the liquid formulation is from about 10 mcg/mL to about 80 mcg/mL. In embodiments of the method, the concentration of diclofenac in the liquid formulation is from about 25 mcg/mL to about 75 mcg/mL. In embodiments of the method, the concentration of diclofenac in the liquid formulation is from about 30 mcg/mL to about 70 mcg/mL. In embodiments of the method, the concentration of diclofenac in the liquid formulation is from about 40 mcg/mL to about 60 mcg/mL. In embodiments of the method, the concentration of diclofenac in the liquid formulation is from about 45 mcg/mL to about 55 mcg/mL. In embodiments of the method, the concentration of diclofenac in the liquid formulation is about 1, 5, 10, 15, 20, 25, 30, 35, 40, 45, 50, 55, 60, 65, 70, 75, 80, 85, 90, 95 or 100 mcg/mL. In embodiments of the method, the concentration of diclofenac in the liquid formulation is about 40 mcg/mL. In embodiments of the method, the concentration of diclofenac in the liquid formulation is about 45 mcg/mL. In embodiments of the method, the concentration of diclofenac in the liquid formulation is about 50 mcg/mL. In embodiments of the method, the concentration of diclofenac in the liquid formulation is about 55 mcg/mL. In embodiments of the method, the concentration of diclofenac in the liquid formulation is about 65 mcg/mL. In embodiments, the local administration of diclofenac is compared to standard human dosing via intravenous infusion of 37.5 mg diclofenac per dose for treatment of lung inflammation.

In embodiments, the disclosure provides a method for treating lung damage or injury, such as from a chemical agent, in a subject, including administering a liquid formulation comprising sodium bicarbonate directly to a lung tissue in the subject; wherein the concentration of the sodium bicarbonate in the liquid formulation is from about 0.5 mg/mL to about 20 mg/mL. In embodiments of the method, the concentration of sodium bicarbonate in the liquid formulation is from about 1 mg/mL to about 19 mg/mL. In embodiments of the method, the concentration of sodium bicarbonate in the liquid formulation is from about 2 mg/mL to about 18 mg/mL. In embodiments of the method, the concentration of sodium bicarbonate in the liquid formulation is from about 3 mg/mL to about 17 mg/mL. In embodiments of the method, the concentration of sodium bicarbonate in the liquid formulation is from about 4 mg/mL to about 16 mg/mL. In embodiments of the method, the concentration of sodium bicarbonate in the liquid formulation is from about 5 mg/mL to about 15 mg/mL. In embodiments of the method, the concentration of sodium bicarbonate in the liquid formulation is from about 8 mg/mL to about 12 mg/mL. In embodiments of the method, the concentration of sodium bicarbonate in the liquid formulation is about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg/mL. In embodiments of the method, the concentration of sodium bicarbonate in the liquid formulation is about 8 mg/mL. In embodiments of the method, the concentration of sodium bicarbonate in the liquid formulation is about 9 mg/mL. In embodiments of the method, the concentration of sodium bicarbonate in the liquid formulation is about 10 mg/mL. In embodiments of the method, the concentration of sodium bicarbonate in the liquid formulation is about 11 mg/mL. In embodiments of the method, the concentration of sodium bicarbonate in the liquid formulation is about 12 mg/mL. In embodiments, the local administration of sodium bicarbonate is compared to standard human dosing via inhalation of 4 mL of a 4.2% solution of sodium bicarbonate per dose for treatment of lung damage.

In embodiments, the disclosure provides a method for treating shock in a subject, including administering a liquid formulation having an agent for treating shock directly to a lung tissue in the subject; wherein the concentration of the agent for treating shock in the liquid formulation is from about 1 mcg/mL to about 200 mcg/mL. In embodiments of the method, the concentration of the agent for treating shock in the liquid formulation is from about 5 mcg/mL to about 190 mcg/mL. In embodiments of the method, the concentration of the agent for treating shock in the liquid formulation is from about 10 mcg/mL to about 180 mcg/mL. In embodiments of the method, the concentration of the agent for treating shock in the liquid formulation is from about 20 mcg/mL to about 170 mcg/mL. In embodiments of the method, the concentration of the agent for treating shock in the liquid formulation is from about 40 mcg/mL to about 160 mcg/mL. In embodiments of the method, the concentration of the agent for treating shock in the liquid formulation is from about 50 mcg/mL to about 150 mcg/mL. In embodiments of the method, the concentration of the agent for treating shock in the liquid formulation is from about 75 mcg/mL to about 125 mcg/mL. In embodiments of the method, the concentration of the agent for treating shock in the liquid formulation is from about 80 mcg/mL to about 120 mcg/mL. In embodiments of the method, the concentration of the agent for treating shock in the liquid formulation is from about 90 mcg/mL to about 110 mcg/mL. In embodiments of the method, the concentration of the agent for treating shock in the liquid formulation is from about 95 mcg/mL to about 105 mcg/mL. In embodiments of the method, the concentration of the agent for treating shock in the liquid formulation is about 0.5, 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mcg/mL. In embodiments of the method, the concentration of the agent for treating shock in the liquid formulation is about 80 mcg/mL. In embodiments of the method, the concentration of the agent for treating shock in the liquid formulation is about 90 mcg/mL. In embodiments of the method, the concentration of the agent for treating shock in the liquid formulation is about 100 mcg/mL. In embodiments of the method, the concentration of the agent for treating shock in the liquid formulation is about 110 mcg/mL. In embodiments of the method, the concentration of the agent for treating shock in the liquid formulation is about 120 mcg/mL. Examples of agents for treating shock are provided herein.

In embodiments, the disclosure provides a method for treating shock in a subject, including administering a liquid formulation comprising dexamethasone sodium phosphate directly to a lung tissue in the subject; wherein the concentration of dexamethasone sodium phosphate in the liquid formulation is from about 1 mcg/mL to about 200 mcg/mL. In embodiments of the method, the concentration of dexamethasone sodium phosphate in the liquid formulation is from about 5 mcg/mL to about 190 mcg/mL. In embodiments of the method, the concentration of dexamethasone sodium phosphate in the liquid formulation is from about 10 mcg/mL to about 180 mcg/mL. In embodiments of the method, the concentration of dexamethasone sodium phosphate in the liquid formulation is from about 20 mcg/mL to about 170 mcg/mL. In embodiments of the method, the concentration of dexamethasone sodium phosphate in the liquid formulation is from about 40 mcg/mL to about 160 mcg/mL. In embodiments of the method, the concentration of dexamethasone sodium phosphate in the liquid formulation is from about 50 mcg/mL to about 150 mcg/mL. In embodiments of the method, the concentration of dexamethasone sodium phosphate in the liquid formulation is from about 75 mcg/mL to about 125 mcg/mL. In embodiments of the method, the concentration of dexamethasone sodium phosphate in the liquid formulation is from about 80 mcg/mL to about 120 mcg/mL. In embodiments of the method, the concentration of dexamethasone sodium phosphate in the liquid formulation is from about 90 mcg/mL to about 110 mcg/mL. In embodiments of the method, the concentration of dexamethasone sodium phosphate in the liquid formulation is from about 95 mcg/mL to about 105 mcg/mL. In embodiments of the method, the concentration of dexamethasone sodium phosphate in the liquid formulation is about 0.5, 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mcg/mL. In embodiments of the method, the concentration of dexamethasone sodium phosphate in the liquid formulation is about 80 mcg/mL. In embodiments of the method, the concentration of dexamethasone sodium phosphate in the liquid formulation is about 90 mcg/mL. In embodiments of the method, the concentration of dexamethasone sodium phosphate in the liquid formulation is about 100 mcg/mL. In embodiments of the method, the concentration of dexamethasone sodium phosphate in the liquid formulation is about 110 mcg/mL. In embodiments of the method, the concentration of dexamethasone sodium phosphate in the liquid formulation is about 120 mcg/mL. In embodiments, the local administration of dexamethasone sodium phosphate is compared to standard human dosing via intravenous infusion of 40 mg of dexamethasone sodium phosphate per dose for treatment of shock.

Drug cocktails are a promising strategy for diseases such as cancer and infections because cocktails can be more effective than individual drugs and can overcome problems of drug resistance. However, finding the best cocktail comprising a given set of drugs is challenging because of the large number of experiments needed, which grows exponentially with the number of drugs. In some embodiments, the disclosure provides a drug cocktail that can be locally delivered by any of the methods of treatment disclosed herein to treat a variety of conditions. In some embodiments, the drug cocktail is a combination of drugs used to treat a disease that would not respond adequately to any of them given alone. In some embodiments, the combination of drugs in the drug cocktail increases the efficacy of treatment of conditions.

In embodiments, provided is a drug cocktail comprising diclofenac, sodium bicarbonate and dexamethasone sodium phosphate at concentrations as provided above. In embodiments, provided is a drug cocktail comprising about 5 to about 100 mcg/mL diclofenac, about 1 to about 20 mg/mL sodium bicarbonate and about 10 to about 200 mcg/mL dexamethasone sodium phosphate. In embodiments, provided is a drug cocktail comprising about 25 to about 75 mcg/mL diclofenac, about 5 to about 15 mg/mL sodium bicarbonate and about 50 to about 150 mcg/mL dexamethasone sodium phosphate. In embodiments, provided is a drug cocktail comprising about 50 mcg/mL diclofenac, about 10 mg/mL sodium bicarbonate and about 100 mcg/mL dexamethasone sodium phosphate. In embodiments, the drug cocktail further comprises albuterol. In embodiments, the drug cocktail further comprises albuterol at a concentration of less than 2 mcg/mL, e.g. about 0.1, 0.5, 1.0 or 1.5 mcg/mL.

In embodiments, the disclosure provides a method of treatment in a subject, wherein the administration of the agent results in a maximum plasma concentration (Cₘₐₓ) of the agent that is very low. In embodiments, the administration results in a plasma Cₘₐₓ for the therapeutic agent that is less than about 60, 70, 80, 90 or 100 ng/mL. In embodiments, the administration results in a plasma Cₘₐₓ for the therapeutic agent that is less than about 50 ng/ml. In embodiments, the administration results in a plasma Cₘₐₓ for the therapeutic agent that is less than about 40 ng/mL. In embodiments, the administration results in a plasma Cₘₐₓ for the therapeutic agent that is less than about 30 ng/mL. In embodiments, the administration results in a plasma Cₘₐₓ for the therapeutic agent that is less than about 29 ng/mL. In embodiments, the administration results in a plasma Cₘₐₓ for the therapeutic agent that is less than about 28 ng/ml. In embodiments, the administration results in a plasma Cₘₐₓ for the therapeutic agent that is less than about 27 ng/mL. In embodiments, the administration results in a plasma Cₘₐₓ for the therapeutic agent that is less than about 26 ng/mL. In embodiments, the administration results in a plasma Cₘₐₓ for the therapeutic agent that is less than about 25 ng/mL. In embodiments, the administration results in a plasma Cₘₐₓ for the therapeutic agent that is less than about 20 ng/ml.

In embodiments, the disclosure provides a method for treating a subject by directly administering a therapeutic agent to the lung tissue of the subject, wherein the plasma Cₘₐₓ of the agent is significantly lower than the plasma Cₘₐₓ that results from systemic administration of the agent (e.g., agent delivered by intravenous administration, parenteral administration or oral administration, etc.). In embodiments, the administration results in a plasma Cₘₐₓ for the therapeutic agent that is less than 20% of the plasma Cₘₐₓ for the therapeutic agent from systemic administration. In embodiments, the administration results in a plasma Cₘₐₓ for the therapeutic agent that is less than 15% of the plasma Cₘₐₓ for the therapeutic agent from systemic administration. In embodiments, the administration results in a plasma Cₘₐₓ for the therapeutic agent that is less than 12% of the plasma Cₘₐₓ for the therapeutic agent from systemic administration. In embodiments, the administration results in a plasma Cₘₐₓ for the therapeutic agent that is less than 6% of the plasma Cₘₐₓ for the therapeutic agent from systemic administration. In embodiments, the administration results in a plasma Cₘₐₓ for the therapeutic agent that is less than 5% of the plasma Cₘₐₓ for the therapeutic agent from systemic administration. In embodiments, the administration results in a plasma Cₘₐₓ for the therapeutic agent that is less than 3% of the plasma Cₘₐₓ for the therapeutic agent from systemic administration. In embodiments, the administration results in a plasma Cₘₐₓ for the therapeutic agent that is less than 1% of the plasma Cₘₐₓ for the therapeutic agent from systemic administration.

In embodiments of the method, the administration results in a plasma Cₘₐₓ for the therapeutic agent that is less than 0.5% of the plasma Cₘₐₓ for the therapeutic agent from systemic administration. In embodiments, the administration results in a plasma Cₘₐₓ for the therapeutic agent that is less than 0.1% of the plasma Cₘₐₓ for the therapeutic agent from systemic administration. In embodiments, the administration results in a plasma Cₘₐₓ for the therapeutic agent that is less than 0.05% of the plasma Cₘₐₓ for the therapeutic agent from systemic administration.

In embodiments, the disclosure provides a method for treating a subject by directly administering a therapeutic agent to the lung tissue of the subject, wherein the plasma Cₘₐₓ of the agent is about 2000-fold lower than the plasma Cₘₐₓ that results from systemic administration of the agent. In embodiments, the plasma Cₘₐₓ of the agent is about 1500-fold lower than the plasma Cₘₐₓ that results from systemic administration of the agent. In embodiments, the plasma Cₘₐₓ of the agent is about 1000-fold lower than the plasma Cₘₐₓ that results from systemic administration of the agent. In embodiments, the plasma Cₘₐₓ of the agent is about 500-fold lower than the plasma Cₘₐₓ that results from systemic administration of the agent. In embodiments, the plasma Cₘₐₓ of the agent is about 250-fold lower than the plasma Cₘₐₓ that results from systemic administration of the agent. In embodiments, the plasma Cₘₐₓ of the agent is about 100-fold lower than the plasma Cₘₐₓ that results from systemic administration of the agent. In embodiments, the plasma Cₘₐₓ of the agent is about 50-fold lower than the plasma Cₘₐₓ that results from systemic administration of the agent. In embodiments, the plasma Cₘₐₓ of the agent is about 10-fold lower than the plasma Cₘₐₓ that results from systemic administration of the agent. In embodiments, the plasma Cₘₐₓ of the agent is about 5-fold lower than the plasma Cₘₐₓ that results from systemic administration of the agent.

In embodiments of the method, the liquid formulation further includes one or more pharmaceutically acceptable excipients suitable for local delivery to lung tissue. In embodiments, the liquid formulation further includes dextrose, sodium chloride, potassium chloride, calcium chloride, sodium bicarbonate or combinations thereof. In embodiments, the liquid formulation further includes saline solution. In embodiments, the liquid formulation further includes Ringer's solution. In embodiments, the liquid formulation has a pH from about 6.0 to about 8.0. In embodiments, the liquid formulation has a pH of about 7.0. In embodiments, the liquid formulation has a pH of about 7.5. In embodiments, the liquid formulation has a pH of about 7.7.

In embodiments, the disclosure provides a method for treating lung cancer in a subject by directly administering a chemotherapeutic agent to the lung tissue of the subject, wherein the chemotherapeutic agent is vinblastine, vinorelbine, irinotecan, paclitaxel, docetaxel, epirubicin, doxorubicin, capecitabine, etoposide, topotecan, pemetrexed, carboplatin, fluorouracil, gemcitabine, oxaliplatin, cisplatin, trastuzumab, ramucirumab, bevacizumab or combinations thereof. In embodiments, the chemotherapeutic agent is paclitaxel, cisplatin, levofloxacin or combinations thereof. In embodiments, the chemotherapeutic agent is paclitaxel. In embodiments, the chemotherapeutic agent is cisplatin. In embodiments, the chemotherapeutic agent is levofloxacin.

In some embodiments, the disclosure provides a method for treating lung damage or injury in a subject by directly administering a drug for inflammation management (e.g., an anti-inflammatory agent) to target tissue of the subject, wherein the drug includes but is not limited to nonsteroidal anti-inflammatory drugs (NSAIDs), such as ibuprofen, naproxen, and diclofenac, and opioids such as fentanyl, hydromorphone, morphine, oxycodone, oxymorphone and tramadol. In some embodiments, the disclosure provides a method for treating shock in a subject by directly administering a drug for shock to target tissue of the subject, wherein the drug for treating shock includes but is not limited to centhaquine, angiotensin II, dopamine, dobutamine, epinephrine, levosimendan, norepinephrine, nitroprusside, nitroglycerine and dexamethasone sodium phosphate. In some embodiments, the anti-inflammatory agent is a corticosteroid. In some embodiments, the corticosteroid is hydrocortisone, hydrocortisone acetate, cortisone acetate, tixocortol pivalate, prednisolone, methylprednisolone, or prednisone. In some embodiments, the corticosteroid is amcinonide, budesonide, desonide, fluocinolone acetonide, fluocinonide, halcinonide, triamcinolone acetonide, Deflazacort beclometasone, betamethasone, dexamethasone, fluocortolone, halometasone, or mometasone. In some embodiments, the corticosteroid is alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.

In some embodiments, the disclosure provides a method for treating lung damage and/or chlorine gas inhalation in a subject by directly administering a drug to lung tissue of the subject, wherein the drug for includes but is not limited to sodium bicarbonate, rituximab, alpha-1 antitrypsin (AAT), nintedanib, pirfenidone, saracatinib, dupixent, warfarin, HER2-directed drugs and mucolytics.

In embodiments, the methods of treating lung damage or injury disclosed are advantageous of prior method as they provide greatly improved treatment and long-term survivability. As an embodiment, the methods provided herein are advantageous because: 1) the initial application of the liquid formulation to the lung tissue flushes debris and toxic or harmful agents from the lungs; 2) the direct contact of the active agent immediately treats the initial symptoms of the lung damage or injury through an application of a high concentration of active agent without the risk for systemic side effects as described elsewhere herein; and 3) the delivery of a active agent can cause a reservoir effect where active agent is maintained in the lung tissue even after the treatment liquid is evacuated. In embodiments, the fact that active agent is maintained in the lung tissue after treatment is especially important to survival of the lung damage or injury, as inflammation, edema and mucous build up following an initial treatment can lead to severe complications or death. When active agent, such an anti-inflammatory agents, are maintained in the lung tissue, these complications after initial treatment can be avoided, leading to improved long term outcomes.

In accordance with an embodiment of the disclosure, a method for administering a therapeutic substance to a target tissue within a hollow organ in a subject is provided that includes forming a treatment chamber having the target tissue in a non-vascular lumen of the hollow organ, heating a liquid formulation, in a holding container, to a treatment temperature during a treatment session, the liquid formulation including the therapeutic substance, and circulating the liquid formulation through the treatment chamber to continually contact the target tissue in the non-vascular lumen of the hollow organ with the liquid formulation during the treatment session.

In accordance with an embodiments of the disclosure, a method for administering a therapeutic substance to a target tissue within a hollow organ in a subject is provided that includes heating a liquid formulation, in a holding container, to a treatment temperature prior to commencement of a treatment session, the liquid formulation having the therapeutic substance, forming a treatment chamber including the target tissue in a non-vascular lumen of the hollow organ, and passively flowing the liquid formulation after heating into the treatment chamber to thereby contact the target tissue in the non-vascular lumen of the hollow organ with the liquid formulation during the treatment session.

In embodiments, the disclosure provides a method for treating a cancer target tissue within a hollow organ of a subject that includes heating a liquid formulation, in a holding container, to a treatment temperature prior to commencement of a treatment session, the liquid formulation having a therapeutic drug; forming a treatment chamber including the cancer target tissue in a non-vascular lumen of the hollow organ; and passively flowing the liquid formulation after heating into the treatment chamber to thereby contact the cancer target tissue in the non-vascular lumen of the hollow organ with the liquid formulation during the treatment session.

In embodiments, the disclosure provides a method for local delivery of a heated liquid drug solution to a target tissue area surrounding a natural lumen extending through an internal body organ of a patient, the method comprising: heating, prior to commencement of a treatment session, a liquid drug solution stored in a holding container, exterior to the patient, to a treatment temperature; inserting a distal region of an elongate flexible catheter through a natural orifice into the natural lumen to a location proximate to the target tissue area; transforming an expandable member on the catheter from a collapsed delivery configuration to an expanded configuration that sealingly engages a wall of the natural lumen proximal to the target tissue area to thereby create a treatment chamber defined by the portion of the natural lumen distal of the expandable member; and circulating the heated liquid drug solution for the duration of the treatment session through the treatment chamber, wherein the step of circulating the heated liquid drug solution comprises permitting air to exit the treatment chamber through an egress lumen extending from a proximal end of the catheter to an egress port located distal of the expandable member, and passively flowing the heated liquid formulation into the treatment chamber.

When the desired drug dosing has been achieved and the treatment session is terminated, the treatment chamber 35 may be evacuated by pumping a flushing fluid therethrough, in similar fashion to the air purging step described above. A non-toxic flushing fluid such as air, saline, or other gases or liquids may be used to clear liquid drug solution 30 from the treatment chamber 35, leaving the flushing fluid therein. Clearing the anti-cancer drug from the treatment chamber 35 may prevent target tissue from being exposed to the drug for a longer time than desired, and/or may prevent non-target tissue from being exposed to the drug when the treatment chamber 35 is broken down by returning expandable members 11, 12 to the collapsed delivery configuration to permit removal of catheter 10 from the patient.

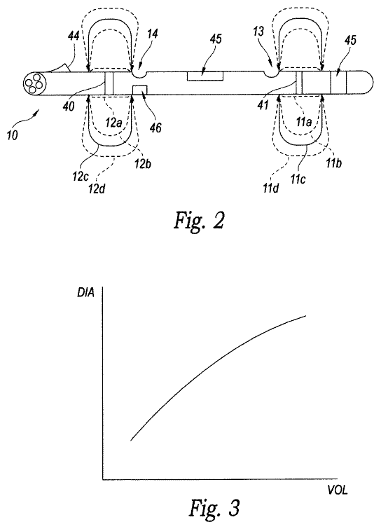

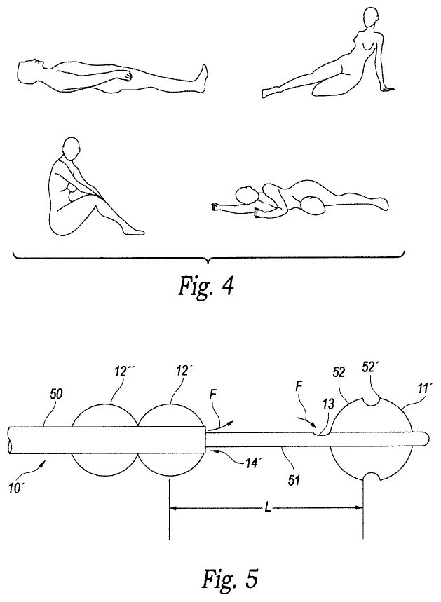

FIG. 2. illustrates a distal region of another catheter 10 configured in accordance with an embodiment of the present technology. Expandable members 11, 12 are shown with variable diameters including fully collapsed respective configurations 11a, 12a, and increasingly larger configurations 11b, 12b; 11c, 12c; and 11d, 12d. The variability in diameter of expandable members 11, 12 allows each member to be selectively expanded into sealing engagement with a natural lumen such as the colon illustrated in FIG. 1. Expandable members 11, 12 may be inflatable clastic balloons, wherein each balloon has a diameter that corresponds in a known relationship to either a gas pressure or a liquid volume, as illustrated in FIG. 3. The selected balloon may be characterized as compliant, non-compliant, elastic or inelastic, depending on its diameter-to-volume or diameter-to-pressure properties. In an example, a catheter having an inelastic balloon may be selected in cases where the known inflated diameter of the balloon can be expected to create an effective treatment chamber 35 seal at the intended anatomic location. In the case of expandable members 11, 12 comprising two balloons, the balloons may be inflated together or separately, to the same or different diameters, and via common or separate inflation lumens 17, 18 shown in FIG. 6, as would be known to those of skill in the field of balloon catheters. Ports 13, 14 are illustrated as being positioned as close to expandable members 11, 12 as possible. The shape of the expanded members 11, 12 and the very adjacent location of the ports thereto can be selected to optimize purging of air from the treatment chamber 35. For example, a balloon may be mounted to catheter 10 with an inverted neck (not shown) to permit locating a port closer to the expandable body of the balloon. Additionally, or alternatively, the expandable member may have a concave or invaginated surface facing towards the treatment chamber 35 to enhance air purging by directing air away from the lumen wall and towards the egress port in the catheter shaft.

The embodiment of catheter 10 shown in FIG. 2 has the following optional features. Fiducial markers 40, 41 may be associated with expandable members 11, 12. In order to assist in locating the treatment chamber 35 with respect to a target area, markers 40, 41 may be visible under medical imaging, e.g., radiopaque markers for visualization under fluoroscopy or sensors (like electromagnetic coils) for use in navigation systems. Orientation sensor 43 may be located proximate the distal region of catheter 10 to inform the operator of the angle of catheter 10 with respect to gravity. The axis of the distal region of catheter 10 is expected to be generally coaxial with the treatment chamber 35 due to the centering effect of expandable members 11, 12. Orientation sensor 43 may be an accelerometer adapted to communicate with an electronic console exterior to the patient.

Orientation sensor 43 may alternatively be an inertial measurement unit (IMU), which is an electronic device that measures and reports an object's specific acceleration, angular rate, and magnetic field surrounding the object, using a combination of accelerometers, gyroscopes, and magnetometers. An IMU works by detecting linear acceleration, rotational rate, and heading reference. When applied to each axis, an IMU can provide pitch, roll, and yaw as well as linear movement. When incorporated into Inertial Navigation Systems, the raw IMU measurement data are utilized to calculate attitude, angular rates, linear velocity and position relative to a global reference frame. IMU data allows a computer to track an object's position, using a method known as dead reckoning or the process of calculating one's current position by using a previously determined position, or fix, and advancing that position based upon known or estimated speeds over elapsed time and course. IMU navigation can suffer accuracy limitations from accumulated error or drift. This error is expected to be reduced in the present technology by combining IMU data with image data generated by camera 44 such that each subsequent image serves as both a new and a cumulative navigational reference. Associating each image frame or a sampling of image frames with a discrete distal IMU pose data point to create a discrete image pose datum is expected to allow navigation errors to be removed.

Camera 44 may be located proximate the distal region of catheter 10 to assist in locating the treatment chamber 35 with respect to a target area. The camera may use optical coherence tomography (OCT) or other small medical camera technologies. Pressure sensor 45 may be located between expandable members 11, 12 to provide data regarding fluid pressure within the treatment chamber 35. The pressure sensor may utilize the piezoelectric effect or other technologies, with the pressure data being useful to monitor and/or maintain safe and effective pressure within the treatment chamber 35 and to potentially detect leakage from the chamber. One or more electrodes 46 may be located between the expandable members and positioned as close as possible thereto. Electrode 46 may be used to monitor electrical impedance, which may be useful to detect when the treatment chamber 35 has filled with liquid or monitor changes in drug concentration.

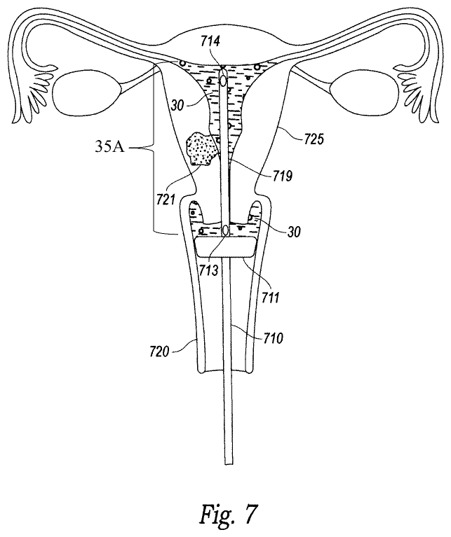

The embodiment of catheter 10′ shown in FIG. 5 has the following optional features. Catheter 10′ comprises a first catheter shaft 50 having an expandable member 12′ mounted at the distal end thereof. A second catheter shaft 51 is slidably disposed within a lumen through shaft 50 and extends distally therefrom. Expandable member 11′ is mounted about a distal region of shaft 51. The operator may adjust how much of shaft 51 extends from shaft 50 to selectively define the length L of the treatment chamber 35 formable between expandable members 11′, 12′. Port 14′ may be an annular clearance space at the terminus of the lumen in shaft 50 that slidably receives shaft 51. Ports 13 and 14′ may function as shown in previous embodiments, including their reversibility, as described above.

Catheter 10′ also comprises a second expandable member 12″ mounted adjacent expandable member 12′ to provide additional sealing capability against a luminal wall beyond that provided by member 12′ alone. This additional, adjacent balloon could serve as a redundant safety feature should sealing of one of the balloons fail. Additional sensors (electrodes, cameras, pressure monitors, etc.) may be placed between these balloons to monitor for fluids indicating a failed seal. Expandable member 11′ comprises multiple lobes 52, 52′ that may also provide additional scaling capability against a luminal wall. A plurality of expandable members, balloons, or lobes may thus be provided to form one or both ends of a treatment chamber (e.g., treatment chamber 35) in accordance with embodiments of the present technology.

FIG. 6 illustrates a drug delivery system 60 configured in accordance with an embodiment of the present technology. Drug delivery system 60 includes catheters 10 or 10′ operably coupled to a console 62. Alternatively, drug delivery system 60 may include other catheters in accordance with the present technology, such as catheters 710, 810, 910, 1010, 1110, 1210, etc., described below. Catheter 10 includes selected features of the catheter embodiments described above, and further includes a flexible elongate shaft 63, a proximal portion 64, and a distal portion 65. Fluid connecting ports 15, 16 are in fluid communication with ports 13, 14 at distal region 65 and may be attached directly or via extension tubes (shown in broken lines) to console 62. Inflation lumens 17, 18 are in fluid communication with expandable members at distal region 65 and may be in communication with separate inflation devices (not shown) or may be connected to console 62 in an embodiment where inflation devices are incorporated therein.

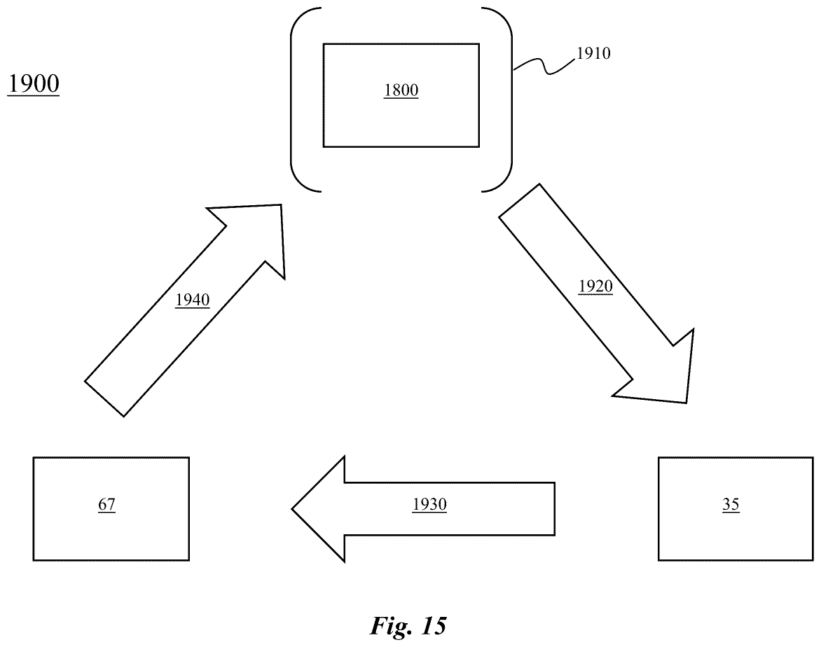

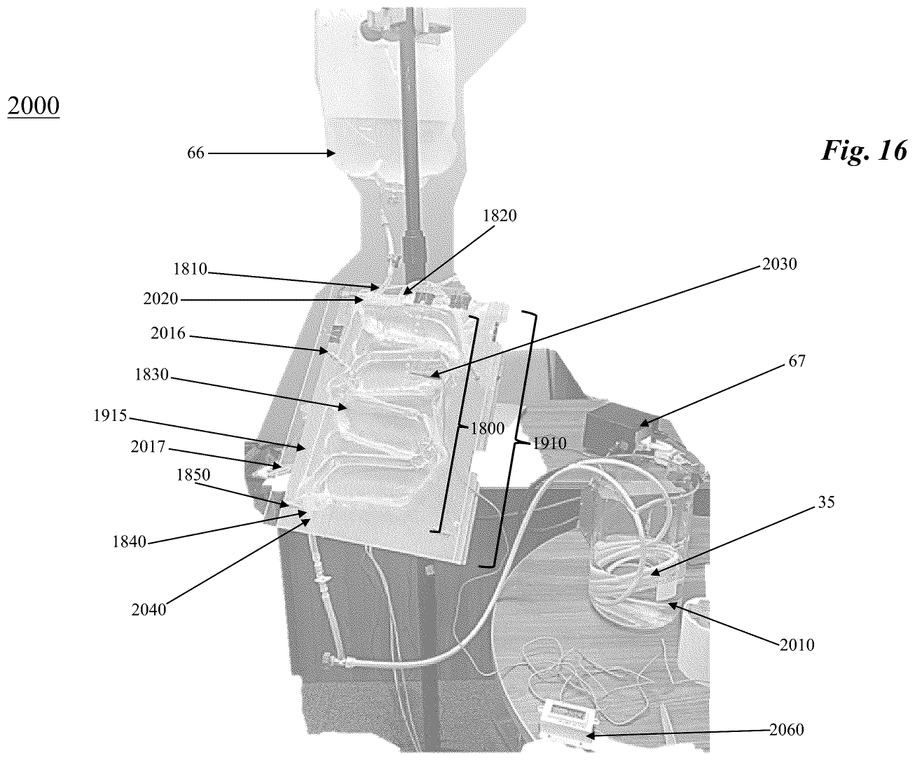

The console 62 may incorporate or be operably coupled to several components adapted to serve different functions as follows. A reservoir 66 (e.g., the holding container 1800 shown in FIG. 14) may contain liquid drug solution 30; a heater 1910 may heat the liquid drug solution 30 in the holding container 1800 (as shown in FIGS. 15 and 16); a pump 67 may recirculate the liquid drug solution 30 via the catheter fluid connecting ports 15, 16; thermometers 72 may monitor the temperature of the liquid drug solution 30; and an osmometer 68 may monitor the concentration of the drug in the circulating liquid drug solution 30. A pressure sensor 69 may electronically communicate with the pressure sensor 45 shown in FIG. 2, or may directly measure pressure in recirculating liquid drug solution 30 within the console 62. Control unit 70 may operate pump 67 based at least in part on one or more inputs selected from measured temperature of therapeutic solution, elapsed time, instantaneous pressure in the closed-loop recirculating fluid circuit, amount of the liquid drug solution 30 added to the fluid circuit, instantaneous drug concentration of the liquid drug solution 30 occupying the closed-loop recirculating fluid circuit, and manual data entered by an operator, e.g., by a keypad 71 on the console 62. The pressure in the closed-loop recirculating fluid circuit may be established, maintained, and changed by the pump 67. For example, the pump 67 may provide a partial vacuum, a.k.a. negative pressure to the egress lumen and egress port to help evacuate the treatment chamber 35 in preparation for administering the liquid drug solution 30 at the beginning of a treatment session or for clearing the treatment chamber 35 of the liquid drug solution 30 at the end of a treatment session. According to an embodiment, the pump 67 is a peristaltic pump. The pump 67 may also maintain pressure of liquid drug solution 30 in the treatment chamber 35 at a selected elevated level, e.g., above atmospheric pressure or above patient blood pressure, to enhance or facilitate uptake of the drug into the target tissue while limiting the selected pressure to avoid injury to tissue or leakage of liquid drug solution 30 past the seal(s) formed by the expandable member(s) at the end(s) of the treatment chamber 35.

Alternatively, the pressure of liquid drug solution 30 in the treatment chamber 35 may be maintained at close to atmospheric pressure by the pump 67, or by a gravity-feed directly from the reservoir 66 (e.g., the holding container 1800) without the use of a pump (thereby passively flowing liquid drug solution 30 into the treatment chamber 35), as illustrated in FIG. 13. Inflation lumens 17, 18 are omitted from FIG. 13 for clarity. During initial fluid filling, most of the isolated chamber is filled with air, which is compressible. As the last of the air is expelled via an egress port when filling nears completion, the fluid in the chamber becomes mostly incompressible liquid. At any time during filling of the chamber, but especially when the liquid medium finishes filling the chamber, a pressure spike may occur therein. The fluid filling rate may exceed the rate at which the chamber can receive the fluid, e.g., the rate at which fluid can be absorbed by tissue or be expelled via an egress port. Such a pressure spike may exceed the scaling pressure in an expandable member, which may allow catheter 10 to shift out of the intended position, or permit leakage of drug solution past an expandable member into unintended areas thereby causing harm to organ tissue. A pressure spike may also cause over-pressure physiologic injury to organ tissue.

Drug delivery system 60 may include a pressure relief circuit 19 that provides a pathway for fluid to escape and thereby avoid compromising pressure spikes in isolated chamber pressures. As shown in FIG. 13, pressure relief circuit 19 is connected via element 82 to a fluid filling line 80 proximal to connector 15. Element 82 may be a simple 3-way T-connector or a 4-way stopcock in a passive system wherein circuit 19 is open to atmosphere at pressure relief circuit end 84. In such a passive system, a relief circuit fluid level 86 in relief circuit 19 will tend to be horizontal with reservoir 66 due to Pascal's Principle. Element 82 may be located anywhere along the fluid channel from reservoir 66, or pump 67 if used, including proximally or distally of connecting port 15 although proximity to distal region 65 improves the responsiveness of pressure relief circuit 19. Element 82 may alternatively be an electronic or mechanical active pressure valve and pressure relief circuit end 84 may be open or may empty into a container. The relief circuit end 84 may include a gas valve or membrane to allow gases to escape, but preventing the liquid from escaping from the pressure relief circuit end 84. Pressure relief circuit 19 can also work during evacuation of the chamber to limit or prevent excessive evacuation forces.

As depicted in FIG. 13, the reservoir 66 for supplying the treatment fluid is elevated above the treatment chamber. A fluid circuit is formed by the reservoir 66, a fluid supply line 80, the catheter 10, and a treatment chamber 35. In some implementations, a waste container may fluidly coupled to the fluid circuit via port 16 of the catheter 10. During operation, treatment fluid (e.g., one or more liquids discussed below) is gravity fed from the elevated reservoir 66 to the treatment chamber 35, filling an entirety of the treatment chamber 35 and expelling any air/gas disposed therein via an egress port 16 of the catheter 10. The pressure relief circuit 19 purges any air from the fluid supply line 80. The purged air exits out to the pressure relief circuit 19 at the circuit end 84. That is, the pressure relief circuit 19 can be used to prime the fluid supply line 80. The pressure relief circuit 19 extends above the fluid level in the elevated reservoir 66. That is, the pressure relief fluid circuit end 84 is disposed above a reservoir fluid level 66A of the elevated reservoir 66. According to Pascal's Principle, the fluid within the pressure relief circuit 19 cannot extend above the reservoir fluid level 66A. Accordingly, the fluid cannot flow past the pressure relief circuit end 84 without external pressure when the pressure relief circuit end 84 is above the reservoir fluid level 66A.

Once primed, and/or as the treatment chamber 35 is filled, the treatment liquid will also flow up the pressure relief circuit 19 according to Pascal's Principal. The treatment fluid is continually supplied from the reservoir 66 until the treatment chamber 35 is completely filled. That is, the fluid pressure from the liquid in the elevated reservoir 66 causes the treatment fluid to fill all the interstitial spaces between tissues of the treatment chamber 35. A difference between the reservoir fluid level 66A and the relief circuit fluid level 86 serves as a barometer for the treatment chamber 35. For example, the relief circuit fluid level 86 being below the reservoir fluid level 66A is an indication that the treatment chamber 35 is not completely filled and that the treatment fluid is continuing to seep into all of the interstitial spaces of the treatment chamber 35 (e.g., all of the free spaces between the tissues of the portion of the organ defining the treatment chamber). Once the relief circuit fluid level 86 in the pressure relief circuit 19 matches the reservoir fluid level 66A in the reservoir 66, the treatment chamber 35 has been fully infused with the treatment fluid. That is, all of the interstitial spaces of the organ making up the fluid chamber 35 is filled with treatment fluid. Accordingly, a user may visually inspect the reservoir 66 and the pressure fluid circuit 19 to determine whether the treatment fluid has completely filled the treatment chamber 35.

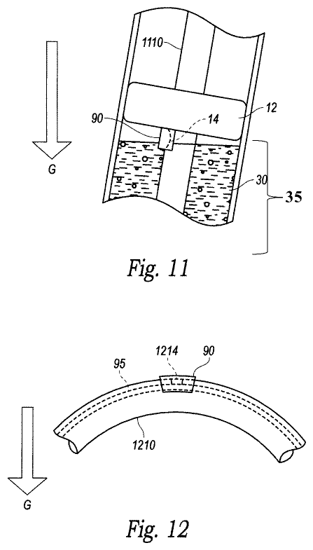

When the treatment chamber 35 is substantially or completely filled with liquid medium, a continued attempt to fill the chamber with liquid can encounter increased resistance, with or without a spike in pressure, resulting in liquid flowing from reservoir 66 being diverted into relief circuit 19. The relief circuit 19 may further serve as a gas vent to avoid fluid lock or air lock within a supply tube 80. The increased resistance to liquid medium flow can be caused by liquid-impermeable membrane 90 covering egress port 14 as described below with respect to the embodiment shown in FIG. 11. Alternatively, the increased resistance to liquid filling when air has been purged from the treatment chamber 35 can be the result of the relatively higher viscosity liquid being forced into the egress lumen. Further, if the operator selects a catheter without an egress feature for purging air from the treatment chamber 35, then an increased resistance to liquid filling can indicate that air in the chamber has been compressed to a small volume, or that air has leaked past an expandable member defining the chamber. The appearance of liquid medium in relief circuit 19 can be a useful indication to the operator that the treatment chamber 35 is full of liquid drug 30 and filling can be stopped. Another useful indication to the operator that the treatment chamber 35 is full of liquid drug solution 30 and filling can be stopped is the appearance of liquid in the egress line(s) associated with e.g., connecting port 16 in FIG. 13.

Furthermore, instead of using a powered pump 67 or gravity feed, liquid drug solution 30 may be circulated through the closed fluid circuit by pushing a fixed volume of liquid drug solution 30 between first and second external reservoirs, e.g., first and second syringes connected to connecting ports 15, 16, as mentioned above.

In some implementations, the drug delivery system 60 may further include a holding container 1800 and a heater 1910 that may be configured to heat a liquid solution held in the holding container 1800, as illustrated in FIG. 15. In certain embodiments, the heater 1910 is thermally coupled (either directly or indirectly) to holding container 1800. For example, in an embodiment, the heater 1910 is thermally coupled directly to the holding container 1800 by a heating circuit (e.g., a flex or flexible heating circuit) coupled to at least one metal plate and the metal plate(s) are directly coupled to the holding container 1800, thereby transferring heat through the direct thermal coupling of the heated metal plates and the holding container 1800. According to another embodiment disclosed herein, the heater 1910 provides microwave heating of the contents of the holding container 1800 such that the heater 1910 need not be thermally coupled to or in direct contact with the holding container 1800. According to other embodiments, the heater 1910 is a flash heater that is configured to heat the liquid drug solution 30.

In embodiments, the heater 1910 heats the liquid drug solution 30 prior to a treatment session or during a treatment session. For example, the heater 1910 may heat the liquid drug solution 30 to a treatment temperature (e.g., between 37° C. and 44° C., such as 42° C.) prior to forming a treatment chamber 35 or any time prior to commencement of the treatment session. According to other embodiments, the heater 1910 may slowly bring the liquid drug solution 30 to the treatment temperature (e.g., between 37° C. and 44° C., such as 42° C.) during the treatment session (e.g., during recirculation, as described above). In embodiments where the heater 1910 heats the liquid drug solution 30, such embodiments may further include monitoring the temperature of the liquid drug solution 30 (e.g., via the thermometers 72 taking measurements and the control unit 70 reading such measurements) and/or maintaining the treatment temperature of the liquid drug solution 30 (e.g., via the control unit 70 adjusting or otherwise controlling the heater 1910 in response to temperature measurements taken by the thermometers 72 and read by the control unit 70).

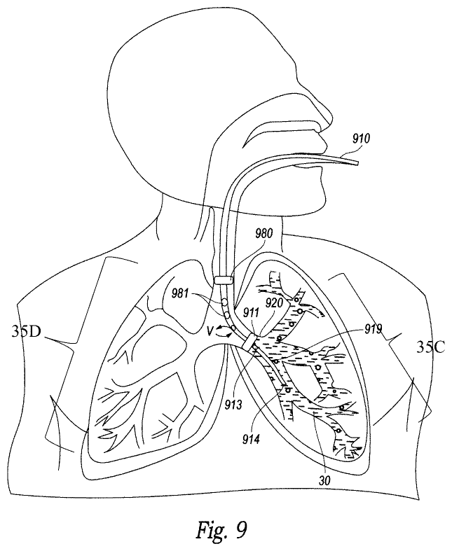

FIG. 7 illustrates a catheter 710 in accordance with another embodiment of the present technology. Catheter 710 is shown in a deployed configuration within a target region of a female genital tract including a portion of a vagina 720 and uterus 725. An expandable member 711 is mounted about a distal region 719 of catheter 710, and is adapted to be expanded into sealing contact with the inner wall of uterus 725, or, as illustrated, with the inner wall of vagina 720. A treatment chamber 35A is defined as the portion of the natural lumen or genital tract distal of expandable member 711. In the illustrated embodiment, the treatment chamber 35A includes a uterine cancer 721. Catheter distal region 719 extends distally of expandable member 711 by a length that may be fixed and selectively pre-designed or that may be variable and selectively adjusted by the operator, similar to length L in catheter 10′ described above and shown in FIG. 5. Port 713 is disposed distally adjacent expandable member 711 and port 714 is disposed at or adjacent the distal end of distal region 719. In the illustrated embodiment, expandable member 711 extends to the fundus of the uterus and thereby positions port 714 at or near the distalmost extent of the desired treatment chamber. Ports 713, 714 fluidly communicate the treatment chamber 35A with respective lumens (not shown) that extend proximally through the catheter to terminate at respective connectors located at the proximal end thereof, comparable to connectors 15, 16 shown in FIG. 6. This embodiment demonstrates a treatment chamber (e.g., treatment chamber 35A) that is defined by the force of gravity and the location of the egress port 714. The proximal end of the treatment chamber 35A may or may not be defined by an expandable member, as described below.

Once catheter 710 has been deployed as shown in FIG. 7, the operator may assess the orientation of the treatment chamber 35A with respect to gravity G, and may reposition the patient, if necessary, to orient the treatment chamber 35A as close to vertical as possible, as described above with respect to catheter 10 in FIG. 1. In the treatment illustrated in FIG. 7, port 714 may be defined as the egress port and port 713 may be defined as the ingress port. After the treatment chamber 35A is oriented with respect to gravity, a liquid drug solution 30 is admitted or forced into the chamber via the ingress port, i.e., port 713 in FIG. 7. As liquid drug solution 30 fills the treatment chamber 35A from bottom to top, air is purged from the chamber via the egress port, i.e., port 714 until the liquid drug solution reaches port 714. In this way, the treatment chamber 35A is filled with liquid drug solution 30, leaving only a small air bubble or preferably no bubble at all. The avoidance of air bubbles or air pockets may ensure that all of the inner wall of female genital tract in the treatment chamber 35 is bathed in liquid drug solution 30. Liquid drug solution 30 may be circulated or recirculated through the treatment chamber 35A between ingress port 713 and egress port 714 as described above with respect to the embodiment shown in FIG. 1. Alternatively, the treatment chamber 35A may be filled with liquid drug solution 30 of a known, e.g., calculated drug concentration for a selected period of time without circulation or recirculation. The treatment chamber 35A may also be evacuated, as described above, by forcing a flushing fluid therethrough using ports 713, 714.

The extent of the treatment chamber 35 formed in the hollow anatomical space may be controlled by limiting the volume or pressure of liquid drug solution 30 admitted or forced into the treatment chamber 35 via catheter 710. In the example illustrated in FIG. 7, liquid drug solution 30 has not been forced into the fallopian tubes from the uterus, although extending the treatment chamber 35A into these spaces may be desirable for treatment of cancer in the fallopian tubes or the ovaries. Since the ostia of the fallopian tubes are proximate to but not in direct connection with the respective ovaries, any liquid drug solution 30 that is forced all the way through one or both fallopian tubes may enter and begin to fill the peritoneal cavity and may result in either intentional or unintentional intraperitoneal therapy. Catheter 710 may be selectively designed or placed such that expandable member 711 creates the proximal end of a treatment chamber 35A in the uterus, or in the vagina, as illustrated. Thus, cancer in various locations throughout the female genital tract may be treated by exposing target tissue to liquid drug solution 30. Alternatively, catheters 10, 10′ may be adapted to create a treatment chamber (e.g., treatment chamber 35) bounded at its ends by two or more expandable members selectively spaced apart and positioned along the female genital tract from the vaginal vestibule, through the cervix, to the fundus of the uterus. In another alternative in accordance with an embodiment of the present technology (not shown), a catheter or catheters may be adapted to operate solely or in combination to create a female genital tract treatment chamber wherein at least distal portions of one or both fallopian tubes are excluded therefrom by expandable member(s) deployed within the respective fallopian tube(s).

Catheter 710 features a single expandable member and two spaced-apart ports disposed distally thereof such that a treatment chamber (e.g., treatment chamber 35A) for use in therapy can be created distally of the expandable member. Although not illustrated, it will be apparent to persons skilled in the relevant art that the scope of the present technology includes catheters, systems and methods wherein two ports are disposed proximally of a single expandable member such that a treatment chamber for use in therapy can be created proximally of the expandable member.

Furthermore, it will be apparent to persons skilled in the relevant art that the scope of the present technology includes catheters, systems and methods wherein a catheter having two spaced-apart ports but without any expandable member can seal within the cervix and thereby form a treatment chamber distally thereof, including the uterus. Such a balloon-less catheter may be a modification of any catheter disclosed herein, for example catheter 10′ of FIG. 5 wherein shaft 50 may be modified to fit sealably within the cervix, especially the cervix of a nulliparous female patient having a very small cervical opening.

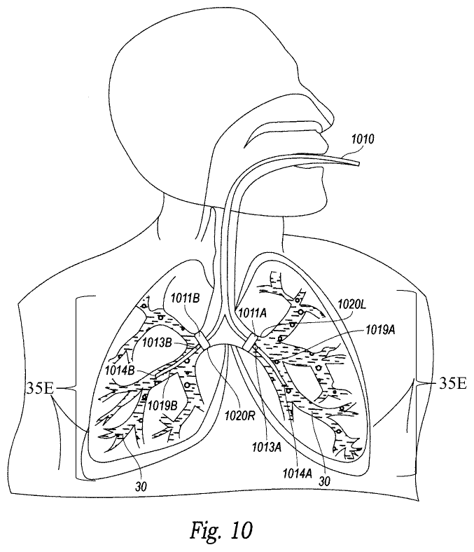

FIG. 8 illustrates a catheter 810 configured in accordance with another embodiment of the present technology. Catheter 810 is shown in a deployed configuration within a target region of a respiratory tract. Catheter distal region 819 extends distally of expandable member 811 by a length that may be fixed and selectively pre-designed or that may be variable and selectively adjusted by the operator, similar to length L in catheter 10′ described above and shown in FIG. 5. Expandable member 811 is mounted about distal region 819 of catheter 810, and is adapted to be expanded into sealing contact with the inner wall of a segmental bronchus 820, as shown. Port 813 is disposed distal and very adjacent expandable member 811 and port 814 is disposed at or adjacent the distal end of distal region 819. A treatment chamber (e.g., treatment chamber 35B) is defined as the portion of the natural lumen or respiratory tract distal of expandable member 811. Catheter 810 may be adapted, if necessary, for creating treatment chambers in different parts of the bronchi of the respiratory tract. The structure and use of catheter 810 are comparable to that of catheter 710 described above and shown in FIG. 7. The patient may be repositioned as described above to optimize orientation of the treatment chamber 35B for purging air therefrom, and ports 813, 814 may be used in similar fashion to ports 13, 14, 713, 714 as described above to purge air and to circulate or recirculate liquid drug solution 30. The treatment chamber 35B may also have air evacuated therefrom before treatment, and/or liquid drug solution cleared out by forcing a flushing fluid therethrough using ports 813, 814.

FIG. 9 illustrates a catheter 910 configured in accordance with another embodiment of the present technology. Catheter 910 is shown in a deployed configuration within a target region of a respiratory tract. Catheter distal region 919 extends distally of expandable member 911 by a length that may be fixed and selectively pre-designed or that may be variable and selectively adjusted by the operator, similar to length L in catheter 10′ described above and shown in FIG. 5. Expandable member 911 is mounted about distal region 919 of catheter 910 and is adapted to be expanded into scaling contact with the inner wall of a main bronchus, e.g., a left main bronchus 920 as shown. Port 913 is disposed distal and very adjacent expandable member 911 and port 914 is disposed at or adjacent the distal end of distal region 919. A treatment chamber (e.g., treatment chamber 35C) is defined as the portion of the natural lumen or respiratory tract distal of expandable member 911. Catheter 910 may be adapted, if necessary, for creating treatment chambers in different parts, e.g., one or more branches of a bronchus of the respiratory tract.

The structure and use of catheter 910 are comparable to those of catheter 810 described above with the addition of inflatable cuff 980 disposed proximally of expandable member 911 such that inflatable cuff 980 can be located above the carina of the trachea. Catheter 910 also has one or more ventilation ports 981 located between inflatable cuff 980 and expandable member 911. Ventilation ports 981 may fluidly communicate with a conventional medical ventilator machine via one or more dedicated lumens (not shown) through catheter 910. While the treatment chamber is bathed in liquid drug solution 30, inflatable cuff 980 may be inflated to seal against the trachea and permit ventilation V of the non-treated lung, e.g., the right lung as shown in FIG. 9, via ventilation ports 981. The patient may be repositioned as described above to optimize orientation of the treatment chamber 35C for purging air therefrom, and ports 913, 914 may be used in similar fashion to ports 13, 14, 813, 814 as described above to purge air and to circulate or recirculate liquid drug solution 30. The treatment chamber 35C may also have air evacuated therefrom before treatment, and/or liquid drug solution cleared out by forcing a flushing fluid therethrough using ports 913, 914. Catheter 910 may be repositioned to permit sequential treatment of left and right respiratory tracts or super-selective treatment of individual bronchi or bronchioles. Alternatively, a similar treatment may be performed by using catheter 810 as described above and simultaneously placing a conventional endotracheal tube such that the inflatable cuff 980 thereof seals around the shaft of catheter 810 and against the trachea to permit ventilation of the non-treated lung.

Catheter 910 may be modified for simultaneous bilateral treatment of the lungs. Instead of ventilation ports 981 being in communication with a ventilator for ventilating the non-treated lung, ports disposed between inflatable cuff 980 and expandable member 911 may be located and connected to perform ingress and egress functions similar to ports 13, 14, 713, 714 as described above to purge air and to circulate or recirculate liquid drug solution 30. Thus, while one treatment chamber (e.g., treatment chamber 35C) receives drug treatment, e.g., a portion of the left lung distal of expandable member 911, the entire respiratory tract of the contralateral lung, e.g., right lung can become a second treatment chamber (e.g., treatment chamber 35D) in fluid communication with the space between inflatable cuff 980 and expandable member 911.

FIG. 10 illustrates a catheter 1010 configured in accordance with another embodiment of the present technology. Bifurcated catheter 1010 is shown in a deployed configuration suitable for simultaneous bilateral treatment of target regions, e.g., left and right, of a respiratory tract. Alternatively, the bilateral treatments may be performed sequentially or overlapping in time without having to replace or reposition catheter 1010. Catheter distal region 1019A of a first catheter branch extends distally of expandable member 1011A by a length that may be fixed and selectively pre-designed or that may be variable and selectively adjusted by the operator, similar to length L in catheter 10′ described above and shown in FIG. 5. Expandable member 1011A is mounted about distal region 1019A of catheter 1010, and is adapted to be expanded into scaling contact with the inner wall of a segmental bronchus 1020L, as shown. Port 1013A is disposed distal and very adjacent expandable member 1011A and port 1014A is disposed at or adjacent the distal end of distal region 1019A. A treatment chamber (e.g., treatment chamber 35E) is defined as the portion of the natural lumen or respiratory tract distal of expandable member 1011A. Similarly, catheter distal region 1019BA of a second catheter branch extends distally of expandable member 1011B by a length that may be fixed and selectively pre-designed or that may be variable and selectively adjusted by the operator, similar to length L in catheter 10′ described above and shown in FIG. 5. Expandable member 1011B is mounted about distal region 1019B of catheter 1010, and is adapted to be expanded into sealing contact with the inner wall of a segmental bronchus 1020R, as shown. Port 1013B is disposed distal and very adjacent expandable member 1011B and port 1014B is disposed at or adjacent the distal end of distal region 1019B. A treatment chamber (e.g., treatment chamber 35E) is defined as the portion of the natural lumen or respiratory tract distal of expandable member 1011B. Catheter 1010 may be adapted, if necessary, for creating treatment chambers in different parts of the bronchi of the respiratory tract. The structure and use of catheter 1010 are comparable to that of catheter 810 described above and shown in FIG. 8 with the addition of a second distal catheter branch. The patient may be repositioned as described above to optimize orientation of the treatment chambers for purging air therefrom, and ports 1013A, 1013B, 1014A, and 1014B may be used in similar fashion to ports 13, 14, 813, 814 as described above to purge air and to circulate or recirculate liquid drug solution 30. The bilateral treatment chambers may also have air evacuated therefrom before treatment, and/or liquid drug solution cleared out by forcing a flushing fluid therethrough using ports 1013A, 1013B, 1014A, and 1014B.

FIG. 11 illustrates a catheter 1110 in accordance with an embodiment of the disclosure wherein exemplary egress port 14 incorporates a membrane 90 to facilitate the procedure of filling a treatment chamber (e.g., treatment chamber 35). Catheter 1110 is otherwise similar to catheter 10, and if the functions of ports 13 and 14 are expected to be reversed, then membrane 90 may be located at port 13 instead of port 14. Membrane 90 is permeable to gases but is impermeable to liquid. For example, membrane 90 will allow air to pass, but not drug solutions for therapy, hormonal therapy or targeted drug/biologic therapy usable with the technology of the disclosure. As described above with regard to the embodiment of FIG. 1, liquid drug solution 30 fills the treatment chamber 35 from the bottom to the top with respect to gravity G, and air is purged from the chamber via the egress port, i.e., port 14 until the liquid drug solution reaches membrane 90 at port 14. See flow arrows F in FIG. 1. Filling the treatment chamber 35 of catheter 1110 via an ingress port 13 can automatically stop when liquid drug solution 30 reaches membrane 90 at port 14. This self-limiting filling feature may be particularly useful for treatments where the drug solution will not be circulated or re-circulated, but instead will remain stationary in the treatment chamber 35 for a duration that is expected to achieve the desired drug dosing. Alternatively, the port on catheter 1110 can serve as the primary air evacuation route as the chamber is filled and liquid may be replaced intermittently by evacuation and refilling via ingress port 13. During evacuation of liquid drug solution 30 from the treatment chamber 35 via ingress port 13, air or other gases may be admitted to the chamber via egress port 14 and membrane 90.

As illustrated in FIG. 6, ports 13, 14 of catheter 10 fluidly communicate the treatment chamber 35 with respective lumens (not shown) that extend proximally through the catheter to terminate at respective connecting ports 15, 16 located at the proximal end thereof. However, the egress port of catheter 1110 need not communicate all the way to the proximal end of catheter 1110. Air or gases purged through membrane 90 and egress port 14 may be exhausted from any point on catheter shaft 10 proximal to the treatment chamber 35. For example, catheter 910 of FIG. 9 may be modified such that egress port 913 may vent purged air through a membrane 90 as shown in FIG. 11 from the treatment chamber 35 through a short catheter lumen that terminates in a modified port 98 located in the patient's trachea. Similarly, for treatment in any natural non-vascular body lumen, gases may be evacuated from the treatment chamber 35 through a gas-permeable membrane and a port located at a chamber high point with respect to gravity, the gases passing through a dedicated vent lumen that terminates in an exhaust port on the catheter located anywhere outside of the treatment chamber 35.

FIG. 12 illustrates a catheter 1210 in accordance with an embodiment of the disclosure wherein the membrane 90 is associated with vent port 1214. Lumen 95 is part of a fluid circuit carrying liquid drug solution through catheter 1210. As shown, catheter 1210 is positioned such that vent port 1214 is located at a high point in catheter 1210 with respect to gravity G. This condition may occur or be caused to occur regardless of where vent port 1214 is located on catheter 1210 outside of the treatment chamber 35. Vent port 1214 may be located anywhere in fluid communication with the fluid circuit, including external connecting lines shown in FIG. 6. Catheter 1210 permits membrane degasification of the liquid drug solution during circulation or recirculation of the drug solution through the treatment chamber 35, a function not provided by catheter 1110. During the purging process, or at any time air or gases are present in lumen 95 proximate to vent port 1214, the air or gases can be expelled via membrane 90 while retaining liquid drug in lumen 95. Membrane degasification of the liquid drug solution requires a pressure gradient across membrane 90, which can be provided by pressurizing the liquid drug solution and/or by vacuating the external surface of membrane 90 to ensure air or gases exit through the membrane 90.

Membrane 90 may comprise a biocompatible porous hydrophobic material such as, but not limited to polypropylene (PP), polytetrafluoroethylene (PTFE), polyvinylidene fluoride (PVDF), or ultra-high molecular weight polyethylene (UHMWPE). Membrane 90 may be adhered or welded to a variety of suitable catheter materials and may form a patch or cover over the egress port or may surround the entire catheter shaft proximate the egress port. Membrane 90 may selectively be applied over any of the ports in the catheter embodiments disclosed herein.

The following chemotherapeutic drugs are considered to be usable with the technology of the disclosure, but are merely given as examples, and not by way of limitation: vinblastine (VELBE), vinorelbin (NAVELBINE), irinotecan (CAMPTOSAR), paclitaxel (TAXOL), docetaxel (TAXOTERE), epirubicin (ELLENCE), doxorubicin (ADRIAMYCIN), capecitabine (XELODA), etoposide (ETOPOPHOS), topotecan (HYCAMTIN), pemetrexed (ALIMTA), carboplatin (PARAPLATIN), fluorouracil (ADRUCIL), gemcitabine (GEMZAR), oxaliplatin (ELOXATIN), cisplatin (PLATINOL), levofloxacin (LEVAQUIN), trastuzumab (HERCEPTIN), ramucirumab (CYRAMZA), and bevacizumab (AVASTIN).

### EXAMPLES

1. A catheter for delivery of a drug to a target tissue area of an internal body organ of a patient, the catheter having an elongate flexible shaft and two longitudinally spaced-apart expandable members disposed about a catheter shaft distal region, the expandable members being transformable between a collapsed delivery configuration and an expanded configuration for sealing against a natural lumen extending through the target tissue area to form a closed treatment chamber defined between the two expandable members and the wall of the natural lumen, the catheter further having first and second drug-delivery lumens extending from a catheter proximal end to respective first and second ports disposed between the expandable members.

2. The catheter of example 1, further comprising an orientation sensor mounted at the catheter shaft distal region.

3. The catheter of any of examples 1 or 2 wherein the two expandable members comprise respectively two compliant balloons wherein each balloon is inflatable to varying diameters, the catheter further having one or more inflation lumens extending from the catheter proximal end to the expandable members for inflating each of the compliant balloons either together or independently.

4. The catheter of any of examples 1-3, further comprising a navigation camera disposed adjacent the distal region.

5. The catheter of any of examples 1-4, further comprising two fiducial markers for referencing the respective locations of the two expandable members when the catheter is viewed using a medical imaging system or a navigation system.

6. The catheter of any of examples 1-5 wherein the two longitudinally spaced-apart expandable members are configured for forming a closed treatment chamber within a lumen of a gastrointestinal tract, a female genital tract, a urinary tract, or a respiratory tract.

7. A catheter for local delivery of a drug to a target tissue area of an internal body organ of a patient, the catheter comprising:
an elongate flexible shaft;
first and second longitudinally spaced-apart expandable members disposed about a distal region of the flexible shaft, the expandable members each being transformable between a collapsed delivery configuration and an expanded configuration for sealing against the wall of a natural lumen extending through the target tissue area to form a closed treatment chamber defined between the first and second expandable members and the wall of the natural lumen;
a liquid ingress lumen extending from a shaft proximal end to a liquid ingress port located between the expandable members; and
a liquid egress lumen extending from a shaft proximal end to a liquid egress port located between the expandable members,
wherein the functions of the ingress and egress ports are reversible such that either port can be a high point of the formed treatment chamber with respect to gravity.

8. The catheter of example 7, further comprising an orientation sensor mounted at the shaft distal region and operable to indicate to an operator the orientation of the shaft distal region with respect to gravity.

9. The catheter of example 8 wherein the orientation sensor is an accelerometer adapted to communicate with an electronic console exterior to the patient.

10. The catheter of any of examples 7-9 wherein the liquid ingress port is located very adjacent the first expandable member and the liquid egress port is located very adjacent the second expandable member.

11. The catheter of any of examples 7-10 wherein both the first and second expandable members are compliant balloons inflatable to varying diameters, the shaft further having one or more inflation lumens configured for inflating the compliant balloons either simultaneously or independently.

12. The catheter of any of examples 7-11, further comprising a navigation camera disposed adjacent the distal region.

13. The catheter of any of examples 7-12, further comprising one or more fiducial markers for referencing the respective locations of the first and second expandable members when the catheter is viewed using a medical imaging system or a navigation system.

14. The catheter of any of examples 7-13 wherein the first and second longitudinally spaced-apart expandable members are configured for forming a closed treatment chamber within a lumen of a gastrointestinal tract, a female genital tract, a urinary tract or a respiratory tract.

15. The catheter of any of examples 7-14, further comprising one or more electrodes disposed between the first and second longitudinally spaced-apart expandable members.

16. The catheter of example 15 wherein the electrodes are configured and located to provide an impedance indication when liquid reaches the high point of the formed treatment chamber with respect to gravity.

17. The catheter of example 15 wherein the electrodes are configured and located to provide an impedance indication of a concentration of the drug in the drug solution.

18. A method for local delivery of a drug to a target tissue area of an internal body organ of a patient, the method comprising:
inserting a distal region of an elongate flexible catheter shaft through a natural orifice into a natural lumen extending through the target tissue area;
transforming two expandable members on the shaft distal region from a collapsed delivery configuration to an expanded configuration in sealing engagement with a wall of the natural lumen to thereby form a closed treatment chamber defined between the two expandable members and the wall of the natural lumen; and
circulating a liquid drug solution for the duration of a treatment session through a closed fluid circuit that comprises the treatment chamber and two drug-delivery lumens that both extend through the catheter shaft from two respective connecting ports exterior to the patient to two respective chamber ports disposed in the shaft distal region between the expandable members.

19. The method of example 18, further comprising:
purging air from the treatment chamber before circulating a liquid drug solution, the purging comprising:
determining the orientation of the shaft distal region with respect to gravity; repositioning the patient, if necessary, such that a one of the chamber ports is located at a high point of the treatment chamber with respect to gravity and defining the port so located as a purge port;
defining the other chamber port located below the purge port in the treatment chamber as a fill port; and
filling the treatment chamber with the liquid drug solution through the fill port while permitting air to exit through the purge port.

20. The method of example 19, further comprising applying negative pressure to the drug-delivery lumen extending from the purge port to enhance purging of air from the treatment chamber.

21. The method of example 19 wherein the purge port is located very adjacent to one of the expandable members.

22. The method of any of examples 18-21, further comprising: terminating the treatment session; and evacuating the treatment chamber of liquid drug solution after terminating the treatment session.

23. The method of any of examples 18-22, further comprising:
measuring a change in a drug concentration in the circulating drug solution over at least a portion of the treatment session;
measuring an elapsed treatment session time; and
calculating an amount of the drug that is dispensed from the treatment chamber based at least in part on the measured change in drug concentration, the measured elapsed treatment session time and a known permeability rate for a given concentration of the drug in a given tissue type.

24. The method of example 23, further comprising terminating the treatment session if the calculated amount of the drug dispensed from the treatment chamber equals or exceeds a predetermined maximum threshold amount.

25. The method of example 23, further comprising terminating the treatment session if the calculated amount of the drug dispensed from the treatment chamber is within a predetermined therapeutic window.

26. The method of example 18, further comprising:
measuring a drug concentration in the circulating drug solution during the treatment session; and
terminating the treatment session if the measured drug concentration is equal to or less than a predetermined minimum threshold amount.

27. The method of example 25 wherein maximum and minimum drug dosage values define the therapeutic window, and the drug dosage values are calculated based at least in part on a desired amount of the drug to be absorbed and an estimated surface area of the wall of the natural lumen in the treatment chamber.

28. The method of example 27 wherein the surface area of the luminal wall in the treatment chamber is estimated based at least in part on one or more of the following parameters:
a known distance between the two expandable members;
a diameter of at least one of the two expandable members;
a distance from the natural orifice of the natural lumen to one of the two expandable members;
an analysis of current and/or previous medical images of the natural lumen extending through the target tissue area of the internal body organ of the patient;
a liquid capacity of the treatment chamber measured when filling the treatment chamber before circulating a liquid drug solution; and
a statistical analysis of historical data regarding physical dimensions of similar natural lumens extending through similar target tissue areas for a known population of patients.

29. The method of example 28 wherein the diameter of at least one of the expandable members is measured from a medical image or the at least one of the expandable members is an inflatable elastic balloon and a diameter of the balloon is determined based at least in part on a volume used to inflate the balloon.

30. The method of examples 28 or 29, further comprising:
estimating the volume of the target tissue area based at least in part on one or more of the following parameters:
   a known distance between the two expandable members;
   a diameter of at least one of the two expandable members;
   a distance from the orifice of the natural lumen to one of the two expandable members;
   an analysis of current and/or previous medical images of the natural lumen extending through the target tissue area of the internal body organ of the patient;
   a liquid capacity of the treatment chamber measured when filling the treatment chamber before recirculating a liquid drug solution; and
   a statistical analysis of historical data regarding physical dimensions of similar natural lumens extending through similar target tissue areas for a known population of patients; and
calculating a desired amount of the circulating liquid drug to be delivered based at least in part on one or more inputs selected from the estimated surface area of the treatment chamber, the estimated volume of the target tissue area, and a known rate of transfer of the drug through the wall of the natural lumen and into the target tissue area.

31. The method of example 23 wherein measuring a change in the drug concentration in the circulating drug solution is performed using an osmometer.

32. The method of any of examples 18-31 wherein circulating the liquid drug solution achieves homogeneous concentration of the drug in the drug solution within in the treatment chamber.

33. The method of any of examples 18-32 wherein transforming two expandable members further comprises adjusting a longitudinal distance between the expandable members such that the length of the closed treatment chamber corresponds with a length of the target tissue area.

34. The method of any of examples 18-33 wherein circulating the liquid drug solution further comprises continuing to circulate the liquid drug solution until the drug has saturated the target tissue area and passed therethrough into the surrounding interstitial space or the proximate lymphatic system of the patient, all of which may act as a conduit or reservoir for the drug.

35. A method for local delivery of a drug to a target tissue area of an internal body organ of a patient, the method comprising:
inserting a distal region of an elongate flexible catheter shaft through a natural orifice into a natural lumen extending through the target tissue area;
transforming two expandable members on the shaft distal region from a collapsed delivery configuration to an expanded configuration in sealing engagement with a wall of the natural lumen to thereby form a closed treatment chamber defined between the two expandable members and the wall of the natural lumen; and
recirculating a liquid drug solution for the duration of a treatment session through a closed-loop fluid circuit that comprises the treatment chamber and two drug-delivery lumens that both extend through the catheter shaft from two respective connecting ports exterior to the patient to two respective chamber ports disposed in the shaft distal region between the expandable members.

36. The method of example 35, further comprising:
purging air from the treatment chamber before recirculating a liquid drug solution, the purging comprising:
determining the orientation of the shaft distal region with respect to gravity; repositioning the patient, if necessary, such that a one of the chamber ports is
located at a high point of the treatment chamber with respect to gravity and defining the port so located as a purge port;
defining the other chamber port located below the purge port in the treatment chamber as a fill port; and
filling the treatment chamber with the liquid drug solution through the fill port while permitting air to exit through the purge port.

37. The method of example 36, further comprising applying negative pressure to the drug-delivery lumen extending from the purge port to enhance purging of air from the treatment chamber.

38. The method of example 36 wherein the purge port is located very adjacent to one of the expandable members.

39. The method of any of examples 35-38, further comprising: terminating the treatment session; and evacuating the treatment chamber of liquid drug solution after terminating the treatment session.

40. The method of any of examples 35-39, further comprising:
measuring a change in a drug concentration in the recirculating drug solution over at least a portion of the treatment session;
measuring an elapsed treatment session time; and
calculating an amount of the drug that is dispensed from the treatment chamber based at least in part on the measured change in drug concentration, the measured elapsed treatment session time and a known permeability rate for a given concentration of the drug in a given tissue type.

41. The method of example 40, further comprising terminating the treatment session if the calculated amount of the drug dispensed from the treatment chamber equals or exceeds a predetermined maximum threshold amount.

42. The method of example 40, further comprising terminating the treatment session if the calculated amount of the drug dispensed from the treatment chamber is within a predetermined therapeutic window.

43. The method of example 35, further comprising:
measuring a drug concentration in the recirculating drug solution during the treatment session; and
terminating the treatment session if the measured drug concentration is equal to or less than a predetermined minimum threshold amount.

44. The method of example 42 wherein maximum and minimum drug dosage values define the therapeutic window, and the drug dosage values are calculated based at least in part on a desired amount of the drug to be absorbed and an estimated surface area of the wall of the natural lumen in the treatment chamber.

45. The method of example 44 wherein the surface area of the luminal wall in the treatment chamber is estimated based at least in part on one or more of the following parameters:
a known distance between the two expandable members;
a diameter of at least one of the two expandable members;
a distance from the orifice of the natural lumen to one of the two expandable members;
an analysis of current and/or previous medical images of the natural lumen extending through the target tissue area of the internal body organ of the patient;
a liquid capacity of the treatment chamber measured when filling the treatment chamber before recirculating a liquid drug solution; and
a statistical analysis of historical data regarding physical dimensions of similar natural lumens extending through similar target tissue areas for a known population of patients.

46. The method of example 45 wherein the diameter of at least one of the expandable members is measured from a medical image or the at least one of the expandable members is an inflatable elastic balloon and a diameter of the balloon is determined based at least in part on a volume used to inflate the balloon.

47. The method of example 45, further comprising:
estimating the volume of the target tissue area based at least in part on one or more of the following parameters:
   a known distance between the two expandable members;
   a diameter of at least one of the two expandable members;
   a distance from the orifice of the natural lumen to one of the two expandable members;
   an analysis of current and/or previous medical images of the natural lumen extending through the target tissue area of the internal body organ of the patient;
   a liquid capacity of the treatment chamber measured when filling the treatment chamber before recirculating a liquid drug solution; and
   a statistical analysis of historical data regarding physical dimensions of similar natural lumens extending through similar target tissue areas for a known population of patients; and
calculating a desired amount of the circulating liquid drug to be delivered based at least in part on one or more inputs selected from the estimated surface area of the treatment chamber, the estimated volume of the target tissue area, and a known rate of transfer of the drug through the wall of the natural lumen and into the target tissue area.

48. The method of example 40 wherein measuring a change in the drug concentration in the recirculating drug solution is performed using an osmometer.

49. The method of example 40 wherein the steps of recirculating a liquid drug solution, measuring a change in a drug concentration in the recirculating drug solution, and calculating the amount of the drug absorbed from the treatment chamber are performed by a system comprising a pump, an osmometer, and a control unit configured to operate the pump based at least in part on one or more inputs selected from elapsed time, instantaneous pressure in the closed-loop fluid circuit, amount of the drug solution added to the fluid circuit, instantaneous drug concentration of the drug solution occupying the closed-loop fluid circuit, and manual data entered by an operator.

50. The method of example 35, further-comprising: monitoring a fluid pressure in the closed-loop fluid circuit.

51. The method of example 50, further comprising maintaining the fluid pressure in the closed-loop fluid circuit within a predetermined pressure range.

52. The method of example 51 wherein the predetermined pressure range includes a positive pressure sufficient to enhance uptake of drug into the target tissue area.

53. The method of example 51 wherein if the monitored fluid pressure exceeds the predetermined pressure range, then a pumping pressure is reduced by a recirculating pump in the closed-loop fluid circuit.

54. The method of example 51 wherein if the monitored fluid pressure is below the predetermined pressure range, then a pumping pressure is increased by a recirculating pump in the closed-loop fluid circuit and/or additional drug solution or solvent is added to the closed-loop fluid circuit.

55. The method of example 50, further comprising terminating the recirculating of a drug solution if a leak in the treatment chamber is indicated by one or more of the following conditions:
the fluid pressure in the closed-loop fluid circuit drops below a predetermined minimum pressure;
a calculated rate of pressure change in the closed-loop fluid exceeds a predetermined rate of change, and
a medical image of the patient shows that one or both of the expandable members is not sufficiently sealing against the wall of the natural lumen.

56. The method of example 50 wherein the fluid pressure in the closed-loop fluid circuit is monitored by a pressure sensor mounted on the catheter in the treatment chamber or a pressure sensor located in an electronic console exterior to the patient and in fluid communication with the closed-loop fluid circuit.

57. The method of example 35, further comprising flushing the drug solution from the closed-loop fluid circuit at the end of the treatment session.

58. The method of example 35 wherein recirculating the liquid drug solution further comprises pumping the liquid drug solution from a pump through one of the two drug-delivery lumens to the treatment chamber while permitting the liquid drug solution to return from the treatment chamber to the pump via the other of the two drug-delivery lumens.

59. A method for local delivery of a liquid drug to a target tissue area surrounding a natural lumen extending through a female genital tract or a respiratory tract or a urinary tract or gastrointestinal tract of a patient, the method comprising:
inserting a distal region of an elongate flexible catheter through a natural orifice into the natural lumen to a location proximate to the target tissue area;
transforming an expandable member on the catheter from a collapsed delivery configuration to an expanded configuration that sealingly engages a wall of the natural lumen proximal to the target tissue area to thereby create a treatment chamber defined by the portion of the natural lumen distal of the expandable member; and
circulating a liquid drug solution for the duration of a treatment session through a closed fluid circuit that comprises the treatment chamber and two drug-delivery lumens that both extend through the catheter shaft from two respective connecting ports exterior to the patient to two respective chamber ports disposed in the shaft region distal of the expandable member.

60. The method of example 59 wherein transforming an expandable member further comprises adjusting a length of the catheter region distal of the expandable member to correspond with a length of the target tissue area.

61. The method of any of examples 59-60 wherein circulating a liquid drug solution comprises delivering a known liquid drug concentration with a known tissue permeability of the drug concentration at a selected flow rate for a selected period of time.

62. The method of any of examples 59-61 wherein circulating a liquid drug solution further comprises pushing a liquid other than the liquid drug through the catheter drug-delivery lumen to force the liquid drug from the catheter drug-delivery lumen into the treatment chamber.

63. The method of any of examples 59-62 wherein the two respective chamber ports in the catheter region distal of the expandable member are longitudinally spaced-apart.

64. The method of example 63, further comprising adjusting the distance that the chamber ports are spaced-apart to correspond with a length of the target tissue area.

65. The method of any of examples 59-64, further comprising evacuating the treatment chamber before circulating a liquid drug solution.

66. The method of any of examples 59-65, further comprising evacuating the treatment chamber of the liquid drug solution after terminating the treatment session.

67. The method of any of examples 59-66, further comprising:
measuring a change in a drug concentration in the circulating drug solution over at least a portion of the treatment session;
measuring an elapsed treatment session time; and
calculating an amount of the drug that is dispensed from the treatment chamber based at least in part on the measured change in drug concentration, the measured elapsed treatment session time and a known permeability rate for a given concentration of the drug in a given tissue type.

68. The method of any of examples 59-67, further comprising terminating the treatment session if the calculated amount of the drug dispensed from the treatment chamber equals or exceeds a predetermined maximum threshold amount.

69. The method of any of examples 59-67, further comprising terminating the treatment session if the calculated amount of the drug dispensed from the treatment chamber is within a predetermined therapeutic window.

70. The method of any of examples 59-67, further comprising:
measuring a drug concentration in the circulating drug dose during the treatment session; and
terminating the treatment session if the measured drug concentration is equal to or less than a predetermined minimum threshold amount.

71. The method of example 69 wherein maximum and minimum drug dosage values define the therapeutic window, and the drug dosage values are calculated before the drug solution is circulated based at least in part on a desired amount of the drug to be absorbed and an estimated surface area of the wall of the natural lumen in the treatment chamber.

72. The method of example 71 wherein the surface area of the luminal wall in the treatment chamber is estimated based at least in part on one or more of the following parameters:
a diameter of the expandable member;
a distance from the orifice of the natural lumen to the expandable member;
a distance from the expandable member to the chamber port located most distally therefrom;
an analysis of current and/or previous medical images of the natural lumen extending through the target tissue area of the internal body organ of the patient;
a liquid capacity of the treatment chamber measured when filling the treatment chamber before circulating a liquid drug solution; and
a statistical analysis of historical data regarding physical dimensions of similar natural lumens extending through similar target tissue areas for a known population of patients.

73. The method of example 72 wherein the diameter of the expandable member is measured from a medical image or the expandable member is an inflatable elastic balloon, and a diameter of the balloon is determined based at least in part on a volume of a fluid used to inflate the balloon.

74. The method of example 72, further comprising:
estimating the volume of the target tissue area based at least in part on one or more of the following parameters:
   a diameter of the expandable member;
   a distance from the orifice of the natural lumen to the expandable member;
   a distance from the expandable member to the chamber port located most distally therefrom;
   an analysis of current and/or previous medical images of the natural lumen extending through the target tissue area of the internal body organ of the patient;
   a liquid capacity of the treatment chamber measured when filling the treatment chamber before circulating a liquid drug solution; and
   a statistical analysis of historical data regarding physical dimensions of similar natural lumens extending through similar target tissue areas for a known population of patients; and
calculating a desired amount of the circulating liquid drug to be delivered based at least in part on one or more inputs selected from the estimated surface area of the treatment chamber, the estimated volume of the target tissue area, and a known rate of transfer of the drug through the wall of the natural lumen and into the target tissue area.

75. The method of example 70 wherein measuring a change in the drug concentration in the circulating drug solution is performed using an osmometer.

76. The method of example 59 wherein circulating a liquid drug solution through a closed fluid circuit further comprises recirculating the liquid drug solution through a closed-loop fluid circuit and the steps of recirculating a liquid drug solution, measuring a change in a drug concentration in the recirculating drug solution, and calculating the amount of the drug absorbed from the treatment chamber are performed by a system comprising a pump, an osmometer, and a control unit configured to operate the pump based at least in part on one or more inputs selected from elapsed time, instantaneous fluid pressure in the closed-loop fluid circuit, amount of the drug solution added to the fluid circuit, instantaneous drug concentration of the drug solution occupying the closed-loop fluid circuit, and manual data entered by an operator.

77. The method of example 76, further comprising monitoring a fluid pressure in the closed-loop fluid circuit.

78. The method of example 77, further comprising maintaining the fluid pressure in the closed-loop fluid circuit within a predetermined pressure range.

79. The method of example 78 wherein the predetermined pressure range includes a positive pressure sufficient to enhance uptake of drug into the target tissue area.

80. The method of example 78 wherein if the monitored fluid pressure exceeds the predetermined pressure range, then a pumping pressure is reduced by the pump in the closed-loop fluid circuit.

81. The method of example 78 wherein if the monitored fluid pressure is below the predetermined pressure range, then a pumping pressure is increased by the pump in the closed-loop fluid circuit and/or additional drug solution or solvent is added to the closed-loop fluid circuit.

82. The method of any of examples 76-81, further comprising terminating the recirculating of a drug solution if a leak in the treatment chamber is indicated by one or more of the following conditions:
the fluid pressure in the closed-loop fluid circuit drops below a predetermined minimum pressure,
a calculated rate of pressure change in the closed-loop fluid exceeds a predetermined rate of change, and
a medical image of the patient shows that one or both of the expandable members is not sufficiently sealing against the wall of the natural lumen.

83. The method of example 77 wherein the fluid pressure in the closed-loop fluid circuit is monitored by a pressure sensor mounted on the catheter in the treatment chamber or a pressure sensor located in an electronic console exterior to the patient and in fluid communication with the closed-loop fluid circuit.

84. The method of any of examples 59-83, further comprising flushing the liquid drug from the closed-loop fluid circuit at the end of the treatment session.

85. The method of any of examples 76-84 wherein recirculating the liquid drug further comprises pumping the liquid drug solution from the pump through one of the two drug-delivery lumens to the treatment chamber while permitting the liquid drug to return from the treatment chamber to the pump via the other of the two drug-delivery lumens.

86. The method of any of examples 59-85 wherein circulating the liquid drug achieves homogeneous concentration of the drug in the liquid drug within in the treatment chamber.

87. The method of any of examples 59-86 wherein circulating the liquid drug further comprises continuing to circulate the liquid drug until the drug has saturated the target tissue area and passed therethrough into the surrounding interstitial space or the proximate lymphatic system of the patient, all of which may act as a conduit or reservoir for the drug.

88. The method of any of examples 59-87 wherein the expandable member is an elastic balloon and predetermined expansion properties thereof comprise a predetermined relationship between inflation volume and diameter.

89. The method of any of examples 59-88 wherein circulating the liquid drug further comprises maintaining a fluid pressure in the treatment chamber below a pre-determined maximum pressure.

90. A catheter for delivery of a drug to a target tissue area of an internal body organ of a patient, the catheter having an elongate flexible shaft and an expandable member disposed about a catheter shaft distal region, the expandable member being transformable between a collapsed delivery configuration and an expanded configuration for sealingly engaging a natural lumen extending through the target tissue area to form a closed treatment chamber defined by the portion of the natural lumen distal of the expandable member, the catheter further having first and second drug-delivery lumens extending from a catheter proximal end to respective first and second ports spaced-apart in the shaft region distal of the expandable members.

91. The catheter of example 90 wherein the proximal port is located very adjacent the expandable member.

92. The catheter of any of examples 90-91 wherein the length between the first and second ports is selectively adjustable to correspond with a length of the target tissue area.

93. The catheter of any of examples 90-92 wherein a length of the catheter region distal of the expandable member is selectively adjustable to correspond with a length of the target tissue area.

94. The catheter of any of examples 90-93 further comprising an orientation sensor mounted at the catheter shaft distal region.

95. The catheter of any of examples 90-94 wherein the expandable member comprises a compliant balloon inflatable to varying diameters, the catheter further having an inflation lumen extending from the catheter proximal end to the expandable member for inflation thereof.

96. The catheter of any of examples 90-95, further comprising a navigation camera disposed adjacent the distal region.

97. The catheter of any of examples 90-96, further comprising a fiducial marker for referencing the location of the expandable member when the catheter is viewed using an imaging system.

98. The catheter of any of examples 90-97 wherein the expandable member is configured for forming a treatment chamber within a lumen of a gastrointestinal tract, a female genital tract, a urinary tract, or a respiratory tract.

99. A catheter for local delivery of a drug to a target tissue area of an internal body organ of a patient, the catheter comprising:
an elongate flexible shaft;
an expandable member disposed about a distal region of the flexible shaft and being transformable between a collapsed delivery configuration and an expanded configuration for sealing against the wall of a natural lumen extending through the target tissue area to form a treatment chamber defined by the wall of the natural lumen distal of the expandable member;
a liquid ingress lumen extending from a shaft proximal end to a liquid ingress port located distal of the expandable member; and
a liquid egress lumen extending from a shaft proximal end to a liquid egress port located distal of the expandable member,
wherein the functions of the ingress and egress ports are reversible such that either port can be a high point of the formed treatment chamber with respect to gravity.

100. The catheter of example 99, further comprising an orientation sensor mounted at the shaft distal region and operable to indicate to an operator the orientation of the shaft distal region with respect to gravity.

101. The catheter of example 100 wherein the orientation sensor is an accelerometer adapted to communicate with an electronic console exterior to the patient.

102. The catheter of any of examples 99-101 wherein one of the liquid ingress port and the liquid egress port is located very adjacent the expandable member.

103. The catheter of any of examples 99-102 wherein a length of the catheter region distal of the expandable member is selectively adjustable to correspond with a length of the target tissue area.

104. The catheter of any of examples 99-103 wherein the expandable member is a compliant balloon inflatable to varying diameters, the shaft further having an inflation lumen configured for inflating the compliant balloon.

105. The catheter of any of examples 99-104, further comprising a navigation camera disposed adjacent the distal region.

106. The catheter of any of examples 99-105, further comprising a fiducial marker for referencing the location of the expandable member when the catheter is viewed using a medical imaging system or navigation system.

107. The catheter of any of examples 99-106 wherein the expandable member is configured for forming a treatment chamber within a lumen of a gastrointestinal tract, a urinary tract, a female reproductive tract, or a respiratory tract.

108. The catheter of any of examples 99-107, further comprising one or more spaced-apart electrodes disposed distally of the expandable member.

109. The catheter of example 108 wherein the electrodes are configured and located to provide an impedance indication when liquid reaches the high point of the formed treatment chamber with respect to gravity.

110. The catheter of example 108 wherein the electrodes are configured and located to provide an impedance indication of a concentration of the drug in the drug solution.

111. A catheter for delivery of a drug to a target tissue area of a lung of a patient, the catheter having:
an elongate flexible shaft and an expandable member disposed about a catheter shaft distal region, the expandable member being transformable between a collapsed delivery configuration and an expanded configuration for sealingly engaging a bronchus extending through the target tissue area to form a closed treatment chamber defined by the portion of the bronchus distal of the expandable member;
first and second drug-delivery lumens extending from a catheter proximal end to respective first and second ports spaced-apart in the shaft region distal of the expandable member;
an inflatable cuff disposed about the catheter shaft proximally of the expandable member and being transformable between a collapsed delivery configuration and an expanded configuration for sealingly engaging a trachea at a location above a tracheal carina of the patient;
one or more ventilation ports located between the cuff and the expandable member; and
a ventilation lumen extending from a catheter proximal end to the one or more ventilation ports.

112. The catheter of example 111 wherein the proximal port is located very adjacent the expandable member.

113. The catheter of any of examples 111-112 wherein the length between the first and second ports is selectively adjustable to correspond with a length of the target tissue area.

114. The catheter of any of examples 111-113 wherein a length of the catheter region distal of the expandable member is selectively adjustable to correspond with a length of the target tissue area.

115. The catheter of any of examples 111-114 further comprising an orientation sensor, an accelerometer, or an IMU mounted at the catheter shaft distal region.

116. The catheter of any of examples 111-116, further comprising a navigation camera disposed adjacent the distal region.

117. The catheter of any of claims 111-116, further comprising at least one port configured for membrane degasification of a liquid drug solution carried by the first and second drug-delivery lumens.

118. The catheter of example 117 wherein the at least one membrane degasification port comprises a membrane that is gas-permeable but is not liquid permeable; and wherein the at least one membrane degasification port is one of the first and second ports distal of the expandable member or the at least one membrane degasification port is located proximal to the expandable member.

119. A catheter for local delivery of a drug to a target tissue area of a lung of a patient, the catheter comprising:
an elongate flexible shaft;
an expandable member disposed about a distal region of the flexible shaft and being transformable between a collapsed delivery configuration and an expanded configuration for sealing against the wall of a bronchus extending through the target tissue area to form a treatment chamber defined by the wall of the bronchus distal of the expandable member;
a liquid ingress lumen extending from a shaft proximal end to a liquid ingress port located distal of the expandable member; and
a liquid egress lumen extending from a shaft proximal end to a liquid egress port located distal of the expandable member; and
an inflatable cuff disposed about the catheter shaft proximally of the expandable member and being transformable between a collapsed delivery configuration and an expanded configuration for sealingly engaging a trachea of the patient;
one or more ventilation ports located between the cuff and the expandable member; and a ventilation lumen extending from a catheter proximal end to the one or more ventilation ports;
wherein the functions of the ingress and egress ports are reversible such that either port can be a high point of the formed treatment chamber with respect to gravity.

120. The catheter of example 119, further comprising an orientation sensor mounted at the shaft distal region and operable to indicate to an operator the orientation of the shaft distal region with respect to gravity.

121. The catheter of example 120 wherein the orientation sensor is an accelerometer or an IMU adapted to communicate with an electronic console exterior to the patient.

122. The catheter of any of examples 119-121 wherein one of the liquid ingress port and the liquid egress port is located very adjacent the expandable member. 123. The catheter of any of examples 119-122 wherein a length of the catheter region distal of the expandable member is selectively adjustable to correspond with a length of the target tissue area.

124. The catheter of any of examples 119-124, further comprising a navigation camera disposed adjacent the distal region.

125. The catheter of any of claims 119-124, further comprising at least one port configured for membrane degasification of a liquid drug solution carried by the ingress and egress lumens.

126. The catheter of example 125 wherein the at least one membrane degasification port comprises a membrane that is gas-permeable but is not liquid permeable; and wherein the at least one membrane degasification port is the egress port or the at least one membrane degasification port is located proximal to the expandable member.

127. The catheter of any of examples 119-126 further comprising one or more spaced-apart electrodes disposed distally of the expandable member.

128. The catheter of example 127 wherein the electrodes are configured and located to provide an impedance indication when liquid reaches the high point of the formed treatment chamber with respect to gravity.

129. The catheter of example 127 wherein the electrodes are configured and located to provide an impedance indication of a concentration of the drug in the drug solution.

130. A method for local delivery of a liquid drug to a target lung tissue area surrounding a bronchus of a patient, the method comprising:
inserting a distal region of an elongate flexible catheter through a natural orifice into a first bronchus to a location proximate to the target tissue area;
transforming an expandable member on the catheter from a collapsed delivery configuration to an expanded configuration that sealingly engages a wall of the first bronchus proximal to the target tissue area to thereby create a treatment chamber defined by the portion of the first bronchus distal of the expandable member;
transforming an inflatable cuff on the catheter from a collapsed delivery configuration to an expanded configuration that sealingly engages a tracheal wall at a location above a tracheal carina of the patient;
circulating a liquid drug solution for the duration of a treatment session through a closed fluid circuit that comprises the treatment chamber and two drug-delivery lumens that both extend through the catheter shaft from two respective connecting ports exterior to the patient to two respective chamber ports disposed in the shaft region distal of the expandable member; and
ventilating a second bronchus opposite the first bronchus through a ventilation lumen extending through the catheter shaft from a respective connecting port exterior to the patient to one or more ventilation ports disposed in the shaft between the inflatable cuff and the expandable member.

131. The method of example 130 wherein transforming an expandable member further comprises adjusting a length of the catheter region distal of the expandable member to correspond with a length of the target tissue area.

132. The method of any of examples 130-131 wherein circulating a liquid drug solution comprises delivering a known liquid drug concentration with a known tissue permeability of the drug concentration at a selected flow rate for a selected period of time.

133. The method of any of examples 130-132 wherein circulating a liquid drug solution further comprises pushing a liquid other than the liquid drug through the catheter drug-delivery lumen to force the liquid drug from the catheter drug-delivery lumen into the treatment chamber.

134. The method of any of examples 130-133 wherein the two respective chamber ports in the catheter region distal of the expandable member are longitudinally spaced-apart.

135. The method of any of examples 130-135, further comprising evacuating the treatment chamber before circulating a liquid drug solution.

136. The method of any of claims 130-135, further comprising degasifying liquid drug in the closed fluid circuit via at least one degasification membrane.

137. The method of example 136 wherein the at least one membrane degasification port is associated with one of the chamber ports, or the at least one membrane degasification port is located proximal to the expandable member.

138. The method of any of examples 130-137, further comprising:
measuring a change in a drug concentration in the circulating drug solution over at least a portion of the treatment session;
measuring an elapsed treatment session time; and
calculating an amount of the drug that is dispensed from the treatment chamber based at least in part on the measured change in drug concentration, the measured elapsed treatment session time and a known permeability rate for a given concentration of the drug in a given tissue type.

139. The method of any of example 138, further comprising terminating the treatment session if the calculated amount of the drug dispensed from the treatment chamber equals or exceeds a predetermined maximum threshold amount.

140. The method of any of example 138, further comprising terminating the treatment session if the calculated amount of the drug dispensed from the treatment chamber is within a predetermined therapeutic window.

141. The method of any of examples 130-137, further comprising:
measuring a drug concentration in the circulating drug dose and/or in the patient's circulating blood during the treatment session; and
terminating the treatment session if the measured drug concentration in the circulating drug dose is equal to or less than a predetermined minimum threshold amount for the circulating drug dose or if the measured drug concentration in the blood is equal to or greater than a predetermined minimum threshold amount in the patient's blood.

142. The method of example 140 wherein the maximum and minimum drug dosage values define the therapeutic window, and the drug dosage values are calculated before the drug solution is circulated based at least in part on a desired amount of the drug to be absorbed and an estimated surface area of the wall of the first bronchus in the treatment chamber.

143. The method of example 142 wherein the surface area of the first bronchus wall in the treatment chamber is estimated based at least in part on one or more of the following parameters:
a diameter of the expandable member;
a distance from the orifice of the natural lumen to the expandable member;
a distance from the expandable member to the chamber port located most distally therefrom;
an analysis of current and/or previous medical images of the first bronchus extending through the target tissue area of the lung of the patient;
a liquid capacity of the treatment chamber measured when filling the treatment chamber before circulating a liquid drug solution; and
a statistical analysis of historical data regarding physical dimensions of similar bronchi extending through similar target tissue areas for a known population of patients.

144. The method of example 143 wherein the diameter of the expandable member is measured from a medical image or the expandable member is an inflatable elastic balloon, and a diameter of the balloon is determined based at least in part on a volume of a fluid used to inflate the balloon.

145. The method of example 143, further comprising:
estimating the volume of the target tissue area based at least in part on one or more of the following parameters:
   a diameter of the expandable member;
   a distance from the orifice of the bronchus to the expandable member;
   a distance from the expandable member to the chamber port located most distally therefrom;
   an analysis of current and/or previous medical images of the bronchus extending through the target lung tissue area of the patient;
   a liquid capacity of the treatment chamber measured when filling the treatment chamber before circulating a liquid drug solution; and
   a statistical analysis of historical data regarding physical dimensions of similar bronchi extending through similar target lung tissue areas for a known population of patients; and
calculating a desired amount of the circulating liquid drug to be delivered based at least in part on one or more inputs selected from the estimated surface area of the treatment chamber, the estimated volume of the target lung tissue area, and a known rate of transfer of the drug through the wall of the bronchus and into the target lung tissue area.

146. The method of example 141 wherein measuring a change in the drug concentration in the circulating drug solution is performed using an osmometer.

147. The method of example 130 wherein circulating a liquid drug solution through a closed fluid circuit further comprises recirculating the liquid drug solution through a closed-loop fluid circuit and the steps of recirculating a liquid drug solution, measuring a change in a drug concentration in the recirculating drug solution, and calculating the amount of the drug absorbed from the treatment chamber are performed by a system comprising a pump, an osmometer, and a control unit configured to operate the pump based at least in part on one or more inputs selected from elapsed time, instantaneous fluid pressure in the closed-loop fluid circuit, amount of the drug solution added to the fluid circuit, instantaneous drug concentration of the drug solution occupying the closed-loop fluid circuit, and manual data entered by an operator.

148. The method of example 147, further comprising monitoring a fluid pressure in the closed-loop fluid circuit.

149. The method of example 148, further comprising maintaining the fluid pressure in the closed-loop fluid circuit within a predetermined pressure range.

150. The method of example 149 wherein the predetermined pressure range includes a positive pressure sufficient to enhance uptake of drug into the target lung tissue area.

151. The method of example 149 wherein if the monitored fluid pressure exceeds the predetermined pressure range, then a pumping pressure is reduced by the pump in the closed-loop fluid circuit.

152. The method of example 149 wherein if the monitored fluid pressure is below the predetermined pressure range, then a pumping pressure is increased by the pump in the closed-loop fluid circuit and/or additional drug solution or solvent is added to the closed-loop fluid circuit.

153. The method of any of examples 147-152, further comprising terminating the recirculating of a drug solution if a leak in the treatment chamber is indicated by one or more of the following conditions:
the fluid pressure in the closed-loop fluid circuit drops below a predetermined minimum pressure,
a calculated rate of pressure change in the closed-loop fluid exceeds a predetermined rate of change, and
a medical image of the patient shows that the expandable member is not sufficiently sealing against the wall of the bronchus.

154. The method of example 148 wherein the fluid pressure in the closed-loop fluid circuit is monitored by a pressure sensor mounted on the catheter in the treatment chamber or a pressure sensor located in an electronic console exterior to the patient and in fluid communication with the closed-loop fluid circuit.

155. The method of any of examples 130-154, further comprising flushing the liquid drug from the closed-loop fluid circuit at the end of the treatment session.

156. The method of any of examples 147-155 wherein recirculating the liquid drug further comprises pumping the liquid drug solution from the pump through one of the two drug-delivery lumens to the treatment chamber while permitting the liquid drug to return from the treatment chamber to the pump via the other of the two drug-delivery lumens.

157. The method of any of examples 130-156 wherein circulating the liquid drug achieves homogeneous concentration of the drug in the liquid drug within in the treatment chamber.

158. The method of any of examples 130-157 wherein circulating the liquid drug further comprises continuing to circulate the liquid drug until the drug has saturated the target lung tissue area and passed therethrough into the surrounding interstitial space or the proximate lymphatic system of the patient, all of which may act as a conduit or reservoir for the drug.

159. The method of any of examples 130-158 wherein the expandable member is an elastic balloon and predetermined expansion properties thereof comprise a predetermined relationship between inflation volume and diameter.

160. The method of any of examples 130-159 wherein circulating the liquid drug further comprises maintaining a fluid pressure in the treatment chamber below a pre-determined maximum pressure.

161. A catheter for bilateral local delivery of a drug to target tissue areas of both lungs of a patient, the catheter comprising:
an elongate bifurcated flexible shaft;
a first expandable member disposed about a first distal branch of the flexible shaft and being transformable between a collapsed delivery configuration and an expanded configuration for sealing against the wall of a first bronchus extending through the first target tissue area to form a treatment chamber defined by the wall of the first bronchus distal of the first expandable member;
a first liquid ingress lumen extending from a shaft proximal end to a first liquid ingress port located distal of the first expandable member; and
a first liquid egress lumen extending from a shaft proximal end to a first liquid egress port located distal of the first expandable member, wherein the functions of the first ingress and egress ports are reversible such that either port can be a high point of the formed treatment chamber with respect to gravity;
a second expandable member disposed about a second distal branch of the flexible shaft and being transformable between a collapsed delivery configuration and an expanded configuration for sealing against the wall of a second bronchus extending through the second target tissue area to form a treatment chamber defined by the wall of the second bronchus distal of the second expandable member;
a second liquid ingress lumen extending from a shaft proximal end to a second liquid ingress port located distal of the second expandable member; and
a second liquid egress lumen extending from a shaft proximal end to a second liquid egress port located distal of the second expandable member, wherein the functions of the second ingress and egress ports are reversible such that either port can be a high point of the formed treatment chamber with respect to gravity.

162. The catheter of example 161, further comprising one or more orientation sensors mounted at the first and/or second shaft distal branch and operable to indicate to an operator the orientation of the respective shaft distal region with respect to gravity.

163. The catheter of example 162 wherein the one or more orientation sensors are accelerometers and/or IMUs adapted to communicate with an electronic console exterior to the patient.

164. The catheter of any of examples 161-163 wherein:
the first liquid ingress port is located very adjacent the first expandable member and the first liquid egress port is spaced distally of the first liquid egress port; and
the second liquid ingress port is located very adjacent the second expandable member and the second liquid egress port is spaced distally of the second liquid egress port.

165. The catheter of any of examples 161-165, further comprising one or more navigation cameras disposed adjacent the first and/or the second distal branches.

166. The catheter of any of claims 161-165, further comprising at least one port configured for membrane degasification of a liquid drug solution carried by either the first or second liquid ingress or egress lumens.

167. The catheter of example 166 wherein the at least one membrane degasification port comprises a membrane that is gas-permeable but is not liquid permeable; and wherein the at least one membrane degasification port is one of the first or second liquid ingress or egress ports or the at least one membrane degasification port is located proximal to the first or second expandable member.

168. The catheter of any of examples 161-167, further comprising one or more electrodes disposed distally of each of the first and second expandable members.

169. The catheter of example 168 wherein the electrodes are configured and located to provide an impedance indication when liquid reaches the high point of each of the formed treatment chambers with respect to gravity.

170. The catheter of example 168 wherein the electrodes are configured and located to provide an impedance indication of a concentration of the drug in the drug solution.

171. A method for bilateral local delivery of a drug to target tissue areas of both lungs of a patient, the method comprising:
inserting a distal region of an elongate bifurcated flexible catheter through a natural orifice such that a first distal catheter branch extends into a first bronchus to a location proximate to a first target tissue area and a second distal catheter branch extends into a second bronchus to a location proximate to a second target tissue area;
transforming an expandable member on the first branch from a collapsed delivery configuration to an expanded configuration that sealingly engages a wall of the first bronchus proximal to the first target tissue area to thereby create a first treatment chamber defined by the portion of the first bronchus distal of the first expandable member;
transforming an expandable member on the second branch from a collapsed delivery configuration to an expanded configuration that sealingly engages a wall of the second bronchus proximal to the second target tissue area to thereby create a second treatment chamber defined by the portion of the second bronchus distal of the second expandable member;
circulating a liquid drug solution for the duration of a treatment session through a first closed fluid circuit that comprises the first treatment chamber and two drug-delivery lumens that both extend through the catheter shaft from two respective connecting ports exterior to the patient to two respective chamber ports disposed in the first shaft region distal of the first expandable member; and
circulating the liquid drug solution for the duration of the treatment session through a second closed fluid circuit that comprises the second treatment chamber and two drug-delivery lumens that both extend through the catheter shaft from two respective connecting ports exterior to the patient to two respective chamber ports disposed in the second shaft region distal of the second expandable member.

172. The method of example 171, further comprising:
purging air from the first and or second treatment chambers before circulating a liquid drug solution, the purging comprising:
determining the orientation of the respective distal catheter branch with respect to gravity;
repositioning the patient, if necessary, such that one of the chamber ports is located at a high point of the respective treatment chamber with respect to gravity and defining the port so located as a purge port;
defining the other chamber port of the respective treatment chamber located below the purge port in the treatment chamber as a fill port; and
filling the respective treatment chamber with the liquid drug solution through the fill port while permitting air to exit through the purge port.

173. The method of example 172, further comprising applying negative pressure to the drug-delivery lumen extending from the defined purge port to enhance purging of air from the treatment chamber.

174. The method of example 172 wherein the defined purge port is located very adjacent to one of the expandable members.

175. A method for local delivery of a liquid drug to a target tissue area surrounding a natural lumen extending through a respiratory tract of a patient, the method comprising:
inserting a distal region of an elongate flexible catheter through a natural orifice into a first bronchus of the patient to a location proximate to the target tissue area;
inserting an endotracheal tube through the natural orifice into the trachea of the patient;
transforming an inflatable cuff on the endotracheal tube from a collapsed delivery configuration to an expanded configuration that sealingly engages the catheter and a tracheal wall at a location above a tracheal carina of the patient; transforming an expandable member on the catheter from a collapsed delivery configuration to an expanded configuration that sealingly engages a wall of the first bronchus proximal to the target tissue area to thereby create a treatment chamber defined by the portion of the first bronchus distal of the expandable member;
ventilating a second bronchus opposite the first bronchus through the endotracheal tube; and
circulating a liquid drug solution for the duration of a treatment session through a closed fluid circuit that comprises the treatment chamber and two drug-delivery lumens that both extend through the catheter shaft from two respective connecting ports exterior to the patient to two respective chamber ports disposed in the shaft region distal of the expandable member.

176. The method of example 175 wherein transforming an expandable member further comprises adjusting a length of the catheter region distal of the expandable member to correspond with a length of the target tissue area.

177. The method of any of examples 175-176 wherein circulating a liquid drug solution comprises delivering a known liquid drug concentration with a known tissue permeability of the drug concentration at a selected flow rate for a selected period of time.

178. The method of any of examples 175-177 wherein circulating a liquid drug solution further comprises pushing a liquid other than the liquid drug through the catheter drug-delivery lumen to force the liquid drug from the catheter drug-delivery lumen into the treatment chamber.

179. The method of any of examples 175-178 wherein the two respective chamber ports in the catheter region distal of the expandable member are longitudinally spaced-apart.

180. The method of any of examples 175-180, further comprising evacuating the treatment chamber before circulating a liquid drug solution.

181. The method of any of claims 175-180, further comprising degasifying liquid drug in the closed fluid circuit via at least one degasification membrane.

182. The method of example 181 wherein the at least one membrane degasification port is associated with one of the chamber ports, or the at least one membrane degasification port is located proximal to the expandable member.

183. A catheter for local delivery of a drug to a target tissue area of an internal body organ of a patient, the catheter comprising:
an elongate flexible shaft;
an expandable member disposed about a distal region of the flexible shaft and being transformable between a collapsed delivery configuration and an expanded configuration for sealing against the wall of a natural lumen extending through the target tissue area to form a treatment chamber defined by the wall of the natural lumen distal of the expandable member;
a liquid ingress lumen extending from a shaft proximal end to a liquid ingress port located distal to the expandable member; and
an egress lumen extending proximally through the shaft from an egress port located distal to the expandable members,
wherein the egress port is covered by a membrane that is permeable by gases but not permeable by a liquid containing the drug.

184. The catheter of example 183 wherein the egress lumen terminates proximally in an exhaust port disposed proximal to both of the expandable member.

185. The catheter of example 183 wherein the egress port is located adjacent to the expandable member to facilitate the egress port being located at a high point of the formed treatment chamber with respect to gravity.

186. The catheter of example 183, further comprising an orientation sensor mounted at the shaft distal region and operable to indicate to an operator the orientation of the shaft distal region with respect to gravity.

187. The catheter of example 186 wherein the orientation sensor is an accelerometer or an IMU adapted to communicate with an electronic console exterior to the patient.

188. The catheter of any of examples 185-187 wherein the egress port is located very adjacent the expandable member.

189. The catheter of any of examples 185-188 wherein the expandable member is a compliant balloon inflatable to varying diameters, the shaft further having an inflation lumen configured for inflating the compliant balloon.

190. The catheter of any of examples 185-189, further comprising a navigation camera disposed adjacent the distal region.

191. The catheter of any of examples 185-190, further comprising a fiducial marker for referencing the location of the expandable member when the catheter is viewed using a medical imaging system or a navigation system.

192. The catheter of any of examples 185-191 wherein the expandable member is configured for forming a closed treatment chamber within a lumen of a gastrointestinal tract, a female genital tract, a urinary tract or a respiratory tract.

193. The catheter of any of examples 185-192, further comprising one or more electrodes disposed distally of the expandable member.

194. The catheter of example 193 wherein the electrodes are configured and located to provide an impedance indication when liquid reaches the high point of the formed treatment chamber with respect to gravity.

195. The catheter of example 193 wherein the electrodes are configured and located to provide an impedance indication of a concentration of the drug in the drug solution.

196. A method for local delivery of a drug to a target tissue area of an internal body organ of a patient, the method comprising:
inserting a distal region of an elongate flexible catheter shaft through a natural orifice into a natural lumen extending through the target tissue area; transforming an expandable member on the shaft distal region from a collapsed delivery configuration to an expanded configuration in sealing engagement with a wall of the natural lumen to thereby form a treatment chamber defined by the portion of the natural lumen distal of the expandable member;
purging air from the treatment chamber, the purging comprising:
determining the orientation of the shaft distal region with respect to gravity; repositioning the patient, if necessary, such that a purge port is located at a high
point of the treatment chamber with respect to gravity; and
filling the treatment chamber with the liquid drug solution through a fill port below the purge port while permitting air to exit the treatment chamber through a porous membrane at the purge port until the porous membrane blocks passage of the liquid drug solution through the purge port; and
holding the liquid drug solution in the treatment chamber for the duration of a treatment session.

197. The method of example 196, further comprising applying negative pressure to the drug-delivery lumen extending from the purge port to enhance purging of air from the treatment chamber.

198. The method of example 196 wherein the purge port is located very adjacent to the expandable member.

199. The method of example 196 wherein the air exiting the treatment chamber through the porous membrane at the purge port is exhausted from the catheter via an exhaust port located proximal to the expandable member.

200. The method of example 196 wherein the air exiting the treatment chamber through the porous membrane at the purge port is exhausted from a portion of the catheter located outside of the patient's body.

201. The method of any of examples 196-200, further comprising: terminating the treatment session; and
evacuating the treatment chamber of liquid drug solution after terminating the treatment session.

202. The method of any of examples 196-201, further comprising:
measuring a drug concentration in the patient's circulating blood during the treatment session; and
terminating the treatment session if the measured drug concentration in the blood is equal to or greater than a predetermined minimum threshold amount in the patient's blood.

203. A method for treating lung damage in a subject, comprising:
administering a liquid formulation comprising an anti-inflammatory agent directly to a lung tissue in the subject,
wherein the concentration of the inflammation relieving agent in the liquid formulation is from about 1 mcg/mL to about 100 mcg/mL.

204. The method of example 203, wherein the concentration of the inflammation relieving agent in the liquid formulation is from about 40 mcg/mL to about 60 mcg/mL.

205. The method of examples 203 or 24, wherein the anti-inflammatory agent is selected from nonsteroidal anti-inflammatory drugs (NSAIDs), such as ibuprofen, naproxen, and diclofenac.

206. The method of examples 203 or 24, wherein the anti-inflammatory agent is diclofenac.

207. A method for treating lung damage, such as from a chemical agent, in a subject, comprising:
administering a liquid formulation comprising sodium bicarbonate directly to a lung tissue in the subject,
wherein the concentration of sodium bicarbonate in the liquid formulation is from about 0.5 mg/mL to about 20 mg/mL.

208. The method of example 207, wherein the concentration of sodium bicarbonate in the liquid formulation is from about 8 mg/mL to about 12 mg/mL.

209. A method for treating shock in a subject, comprising:
administering a liquid formulation comprising an agent for treating inflammation directly to a lung tissue in the subject,
wherein the concentration of the anti-inflammatory agent in the liquid formulation is from about 1 mcg/mL to about 200 mcg/mL.

210. The method of example 209, wherein the concentration of the agent for treating lung inflammation in the liquid formulation is from about 90 mcg/mL to about 110 mcg/mL.

211. The method of example 209 or 210, wherein the agent for treating shock is selected from centhaquine, angiotensin II, dopamine, dobutamine, epinephrine, levosimendan, norepinephrine, nitroprusside, nitroglycerine and dexamethasone sodium phosphate.

212. The method of example 209 or 210, wherein the agent for treating inflammation shock is dexamethasone sodium phosphate.

213. A drug cocktail comprising about 5 to about 100 mcg/mL diclofenac, about 1 to about 20 mg/mL sodium bicarbonate and about 10 to about 200 mcg/mL dexamethasone sodium phosphate.

214. A drug cocktail comprising about 25 to about 75 mcg/mL diclofenac, about 5 to about 15 mg/mL sodium bicarbonate and about 50 to about 150 mcg/mL dexamethasone sodium phosphate.

215. A drug cocktail comprising about 50 mcg/mL diclofenac, about 10 mg/mL sodium bicarbonate and about 100 mcg/mL dexamethasone sodium phosphate.

216. The drug cocktail of examples 213-215, further comprising albuterol.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described exemplary embodiments but should be defined only in accordance with the appended claims and their equivalents. It will also be apparent that all hollow organs are eligible for both single and multiple balloon configurations of the devices, systems and methods described herein. It will also be understood that each feature of each embodiment discussed herein, and of each reference cited herein, can be used in combination with the features of any other embodiment.

### Example A—Local Diclofenac Drug Delivery to the Lungs in a Porcine Model

Studies were performed using a porcine model to study the distribution of diclofenac in the system when administered locally (via direct administration to the lungs).

The purpose of the study was to determine the systemic concentration of diclofenac after a maximal 30 minute local lavage, taking samples at specific time points for up to 30 minutes.

Endpoints to be evaluated are:
Endpoint 1: overall animal health (moribundity)
Endpoint 2: tissue response to treatment
Endpoint 3: diclofenac analysis

Diclofenac is a nonsteroidal anti-inflammatory drug (NSAID) used to treat mild-to-moderate pain, and helps to relieve symptoms of arthritis (eg, osteoarthritis or rheumatoid arthritis), such as inflammation, swelling, stiffness, and joint pain. The chemical name is 2-[2-(2,6-dichloroanilino)phenyl]acetic acid. Diclofenac sodium is a white or slightly yellowish crystalline powder and is sparingly soluble in water at 25° C. with the molecular formula C₁₄H₁₀Cl₁₂NNaO₂ and a molecular weight of 318.14 g/mol. The standard dosage of Diclofenac in an intravenous solution for humans is 37.5 mg/mL.

Animals tested were of the porcine species from the breed Yorkshire Cross. The animals underwent surgery to place two (2) CVL catheters, 1 for infusion and 1 for plasma collection. Each animal received ˜1 L of lavage solution. The animals were allowed to recover and stabilize. Once the animals were awake and mobile, the animals were infused with 50 mcg/mL for 30 minutes. The animal had blood drawn on baseline, every 5 minutes during infusion, and approximately 20 minutes after the start of Diclofenac infusion followed by termination and visual analysis of the animal tissue. The plasma concentrations of diclofenac following local administration in swine are summarized in Table 1.

TABLE 1
Diclofenac Plasma Concentration Following
Administration in Test Animals

| | | Plasma Concentration (ng/mL) by Time (min) | | | |
|---|---|---|---|---|---|
| | Animal ID | 0 min | 5 min | 15 min | 30 min |
| Drug | 24P0133 | BLQ | 3430 ng/mL | 3050 ng/mL | 2410 ng/mL |

BLQ: Below Limit of Quantitation

For all concentration measurements herein, the sample extracts were analyzed for diclofenac by liquid chromatography/mass spectrometry (LC/MS). Spectra were compared to the validated protocol and standard curve generated for the study.

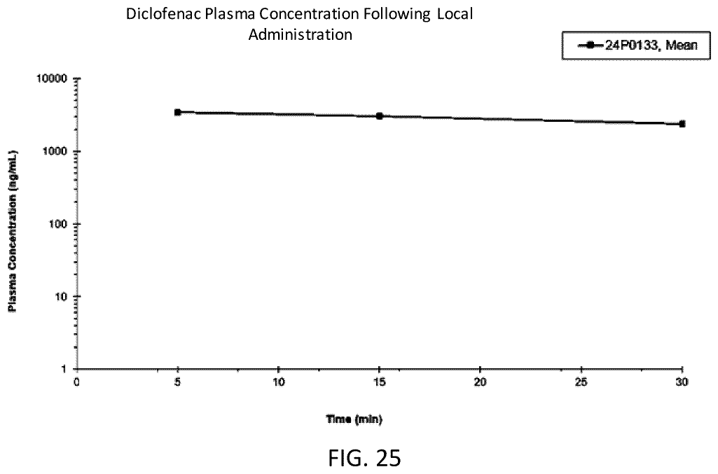

A plot of the average concentrations in the samples is shown in FIG. 25.

### Discussion/Conclusions

This diclofenac study focused on the concentration of drug in the plasma of porcine model when the drug is delivered locally.

The animals for the local drug delivery did not have any adverse events and had no abnormal clinical observations and demonstrated no signs of lung tissue damage.

The diclofenac concentration used for local lung delivery at 50 mcg/mL had a peak plasma concentration at 5 minutes post-lung infusion.

Local administration achieves equivalent levels of delivery of diclofenac to the lung tissue while having a plasma Cₘₐₓ of less than 0.05% of the plasma Cₘₐₓ resulting from systemic administration. Local administration thus leads to a plasma Cₘₐₓ that is more than 2000 fold lower than the plasma Cₘₐₓ with systemic administration. This very low level of diclofenac circulating through the animal's blood is very likely to be the reason why few side effects were seen in the animals when receiving local administration.

Animals receiving local administration had less abnormal physical observations and lower effects to the overall system as shown by plasma concentrations.

### Example B—Porcine Local Administration Studies with Dexamethasone Sodium Phosphate

Dexamethasone sodium phosphate is a sodium phosphate salt form of dexamethasone, a synthetic adrenal corticosteroid with potent anti-inflammatory properties. In addition to binding to specific nuclear steroid receptors, dexamethasone also interferes with NF-kB activation and apoptotic pathways and lacks the salt-retaining properties of other related adrenal hormones. The chemical name is disodium; [2-[(8S,9R,10S,11S,13S,14S,16R, 17R)-9-fluoro-11,17-dihydroxy-10,13,16-trimethyl-3-oxo-6,7,8, 11, 12, 14, 15, 16-octahydrocyclopenta[a]phenanthren-17-yl]-2-oxoethyl] phosphate. Dexamethasone sodium phosphate is freely soluble in water (100-1000 mg/mL) is exceedingly hygroscopic with the molecular formula C₂₂H₂₈FNa₂O₈P and a molecular weight of 516.4 g/mol. Dexamethasone Sodium Phosphate Injection is typically administered in a sterile solution for intravenous and intramuscular use. The standard dosage of Diclofenac in an intravenous solution for humans is 40 mg/mL.

Porcine studies were performed similarly to as described in Example A but using dexamethasone sodium phosphate as the drug agent locally administered to the lung. The animals that received local drug delivery of dexamethasone sodium phosphate did not have any adverse events and had no abnormal clinical observations and demonstrated no signs of lung tissue damage. The dexamethasone sodium phosphate was well tolerated with no signs of localized lung damage in the animals. This suggests that local administration of Dexamethasone sodium phosphate results in minimal systemic exposure.

### Example C—Porcine Local Administration Studies with Sodium Bicarbonate

Sodium bicarbonate is a salt that breaks down to form sodium and bicarbonate in water and is able to neutralize acid. Sodium bicarbonate's ability to neutralize acid helps treat conditions related to high acidity in bodily fluids, such as indigestion, which is caused by too much acid in the stomach. Nebulized sodium bicarbonate can be used to treat chlorine gas inhalation by neutralizing hydrochloric acid that forms when chlorine reacts with water in the respiratory system. Inhaled sodium bicarbonate can neutralize hypochlorous and hydrochloric acids, decreasing severity of lung damage or injury. Sodium bicarbonate is typically administered in a 4.2% solution for inhalation in humans.

Porcine studies with local administration to the lung were performed similarly to as described in Example A but using sodium bicarbonate as the therapeutic agent. Sodium bicarbonate was locally administered to test animals via infusion with 10 mg/mL for 30 minutes. Due to the nature of sodium bicarbonate, plasma concentration was not measured in test animals. The animals that received local drug delivery of sodium bicarbonate did not have any adverse events, had no abnormal clinical observations and demonstrated no signs of lung tissue damage.

### Drug Cocktail

Based on the above testing of individual drugs in animals, a drug “cocktail” combination that can be used in the local drug delivery to tissue was developed. The drug cocktail combination and concentrations of the individual drugs in the combination are summarized in Table 2.

TABLE 2

| Drug | Cocktail concentration | |
|---|---|---|
| Diclofenac | 50 | mcg/mL |
| Sodium bicarbonate | 10 | mg/mL |
| Dexamethasone sodium phosphate | 100 | mcg/mL |

The experiments described in Examples A, B, and C show how traditional therapeutic agents can be used for local administration without any of the complications and constraints that come with using these agents systemically. The methods described herein thus open new avenues for effective treatment of lung cancer using well characterized, highly effective, therapeutic agents.

As described above, embodiments described herein include systems and methods for delivery of a therapeutic solution to a target tissue area of an internal body organ. Such methods and systems provide for effective treatment of various diseases and are described in further detail below.

FIG. 14 is an illustration of a holding container 1800 in accordance with an embodiment of the disclosure. As shown in FIG. 14, the holding container 1800 includes two inlet ports (e.g., inlet ports 1810 and 1820) fluidly coupled to a lumen 1830. According to embodiments herein, either the inlet port 1810 and/or the inlet port 1820 may optionally be configured to allow entry of an initial drug solution, stored in reservoir 66 (e.g., liquid drug solution 30 stored at either room or ambient temperature) into the holding container 1800 and/or configured as a return for the circulated fluid (e.g., liquid drug solution 30) from the treatment chamber 35. Additionally, either the inlet port 1810 and/or the inlet port 1820 may optionally be configured to allow additional drug solution (e.g., liquid drug solution 30) to be added during a treatment session; e.g., to top off the holding container 1800 after delivery to the treatment chamber 35 commences. Moreover, either the inlet port 1810 and/or the inlet port 1820 may optionally be configured to allow saline to flush the drug delivery system 60 and the treatment chamber 35 after completion of a treatment session. Although FIG. 14 shows the holding container 1800 with two inlet ports, the positions of the inlet port 1810 and the inlet port 1820 may be swapped according to embodiments herein. Moreover, according to embodiments, the inlet port 1810 and the inlet port 1820 may be combined into a single inlet port (not shown) such that the holding container 1800 includes a single inlet port that may be in fluid communication with a T-port. As shown in FIG. 14, lumen 1830 generally forms a pathway that curves back and forth along a length of the holding container. Lumen 1830 is not limited to such a configuration and may form a pathway-such as an elongated zig-zag or up and down configuration. The lumen 1830, in turn, is fluidly coupled to an outlet port 1840. Stated another way, the lumen 1830 extends between the inlet ports 1810, 1820 and the outlet port 1840. The holding container 1800 includes a plurality of holding brackets 1850 each of which is configured to receive an anchoring mechanism (not shown) to keep the holding container 1800 in place during a treatment session. According to embodiments, the holding container 1800 is configured to permit the liquid drug solution 30 held therein to be safely and effectively heated to a treatment temperature. According to embodiments, the holding container 1800 is constructed of polyvinyl chloride (PVC), Ethylene-vinyl acetate (EVA), polypropylene (PP), polyethylene (PE) or polyolefin blend (PP+PE), Polyurethane (PU), polybutadiene (PBD), or any other plastic material suitable to safely and effectively heat liquid drug solution 30 to the treatment temperature. Moreover, according to embodiments herein, lumen 1830 includes a bag liner (not shown) and that bag liner that may be joined at the seams to the holding container 1800 with a heat seal (ultrasonic welded, induction welding, etc.), adhesives or other suitable sealing mechanism. According to embodiments herein, the shape of the lumen 1830 allows for an even distribution of heat applied to the holding container 1800 (e.g., via the heater 1910). By evenly distributing heat, the lumen 1830 beneficially accelerates the heating of the liquid drug solution 30 compared to a conventional holding container. According to certain embodiments, the holding container 1800 is further suited for safely and effectively holding the heated liquid drug solution 30 for a specific holding period, without damage to the holding container 1800. For example, the holding container 1800 may safely and effectively hold and/or bring the heated liquid drug solution 30 up to the treatment temperature for a holding period of approximately 10 minutes. While not shown in FIG. 14, the holding container 1800 may by thermally coupled to various temperature gauges (e.g., thermometers 72) to measure the temperature of the liquid drug solution 30 as it enters an inlet port (e.g., cither the inlet port 1810 or the inlet port 1820), traverses the lumen 1830 or exits via the outlet port 1840. According to embodiments, the holding container 1800 holds 0.25 to 1.0 liter of fluid and may contain between 0.25 and 1.0 liter of the liquid drug solution 30.

FIG. 15 is a schematic illustration of a treatment system in accordance with an embodiment of the disclosure. The treatment system of FIG. 15 illustrates local administration of a heated solution and may be configured to include one or more of the components described above (e.g., components described above for the drug delivery system 60). As shown in FIG. 15, treatment system 1900 includes a heater 1910 that is thermally coupled to a holding container 1800, to thereby heat a fluid in the holding container 1800. As described above, the heater 1910 optionally includes a flex heating circuit attached to at least one metal plate. According to other embodiments described herein, the heater 1910 is a flash heater and optionally heats the fluid in the holding container 1800 via microwaves directed at a fluid in the holding container 1800. As described above, according to embodiments, a therapeutic solution (e.g., liquid drug solution 30) is held in the holding container 1800 and heated to a treatment temperature (e.g., between 37° C. and 44° C., such as 42° C.) prior to commencement of the treatment session. Optionally, a therapeutic solution is held in the holding container 1800 for a certain amount of time (e.g., a holding period). In certain embodiments, the holding period is approximately 10 minutes. According to certain embodiments, the holding period takes place prior to commencement of the treatment session. Moreover, according to embodiments, the therapeutic solution may be held in the holding container 1800 and slowly heated from a base or ambient temperature to a treatment temperature (e.g., between 37° C. and 44° C., such as 42° C.). In certain embodiments, the therapeutic solution is slowly heated from a base or ambient temperature to a treatment temperature while the therapeutic solution is circulated or recirculated.

As schematically shown in FIG. 15, holding container 1800 is in fluid communication, via a first fluid communication channel 1920 (e.g., tubing, etc.), with the treatment chamber 35. According to embodiments, the therapeutic solution being held in the holding container 1800 passively flows into the treatment chamber 35. More particularly, the therapeutic solution being held in the holding container 1800 may be gravity fed into the treatment chamber 35. Alternatively, while not shown in FIG. 15, positive pressure may be used to push the therapeutic solution being held in the holding container 1800 into the treatment chamber 35. Schematically, FIG. 15 further shows the treatment chamber 35 being in fluid communication, e.g., via a second fluid communication channel 1930 (e.g., tubing, etc.), with a pump 67. As described above, the pump 67 may exert either positive or negative pressure; however, in the embodiment shown in FIG. 15, pump 67 exerts negative pressure to permit the withdrawal of the therapeutic solution from the treatment chamber 35.

According to the embodiment of treatment system 1900 schematically shown in FIG. 15, pump 67 is in fluid communication, via a third fluid communication channel 1940 (e.g., tubing, etc.), with the holding container 1800. As described above, the pump 67 may exert either positive pressure (e.g., pushing a fluid into the treatment chamber 35, not shown in FIG. 15) or negative pressure (e.g., pulling a fluid from the treatment chamber 35, as shown in FIG. 15). In either embodiment, the pump 67 is situated between the holding container 1800 and the treatment chamber 35. Furthermore, according to certain embodiments, the pump 67 is a peristaltic pump. In the embodiment shown in FIG. 15, the holding container 1800, the treatment chamber 35 and the pump 67 are in fluid communication, e.g., via the first fluid communication channel 1920, the second fluid communication channel 1930, and the third fluid communication channel 1940, with one another, thereby forming a closed fluid circuit.

FIG. 16 is an illustration of a treatment system 2000 in accordance with another embodiment of the disclosure. The treatment system 2000 of FIG. 16 illustrates local administration of a heated solution with temperature monitoring and may be configured to include one or more of the components described above (e.g., components described above for the drug delivery system 60). As shown in FIG. 16, the treatment system 2000 includes a reservoir 66 that is in fluid communication with the holding container 1800 via inlet port 1810. The holding container 1800 is thermally coupled to the heater 1910 via a direct coupling between the holding container 1800 and a metal plate 1915 of the heater 1910. While not shown, the metal plate 1915 is heated to operating temperature by a flex heating circuit that optionally includes a silicone heater pad. According to one embodiment, the metal plate 1915 is an aluminum plate and the metal plate 1915 is heated to an operating temperature via a silicone heater pad coupled to the metal plate 1915. While not shown in FIG. 16, the heater 1910 is controlled by the control unit 70 (e.g., a temperature controller) and the control unit 70 receives temperature input from the heater 1910 via a thermistor coupled to silicone heater pad. The direct thermal coupling of the heater 1910 to the holding container 1800 thereby heats a fluid in the holding container 1800. In FIG. 16, direct thermal coupling of the heater 1910 to the holding container 1800 is assisted by a holding plate 2017, where the holding plate 2017 improves contact of the holding container 1800 with the heater 1910 by pressing the holding container 1800 against the metal plate 1915. Optionally, the holding plate 2017 is securely fastened to the holding container 1800 by a plurality of anchoring mechanisms 2016 coupled to a plurality of corresponding holding brackets 1850 that together keep the holding container 1800 in place during a treatment session. According to one embodiment, the holding plate 2017 is an acrylic plate. As further shown in FIG. 16, the holding container 1800 is in fluid communication with the treatment chamber 35 via the outlet port 1840. The treatment chamber 35 is in fluid communication with the pump 67, which is in fluid communication with the holding container 1800 via the inlet port 1820. Moreover, as shown in FIG. 16, a closed fluid circuit is formed by the holding container 1800, the treatment chamber 35 and the pump 67 being in fluid communication with one another. FIG. 16 further illustrates temperature monitoring via temperature gauges 2010, 2020, 2030 and 2040, where the temperature gauge 2010 is located in the treatment chamber 35 (or a simulated treatment chamber), the temperature gauge 2020 is located near the inlet ports 1810 and 1820, the temperature gauge 2030 is located inside the lumen 1830 and the temperature gauge 2040 is located near the outlet port 1840. According to one embodiment, temperature gauges 2010, 2020, 2030 and 2040 are thermocouples (e.g., NTC thermocouples) coupled to the control unit 70 and a data acquisition device (not shown). Optionally, temperature gauges 2010, 2020, 2030 and 2040 are calibrated by comparing their respective output to a calibrated thermocouple. According to embodiments, temperature gauges 2010, 2020, 2030 and 2040 optionally include at least one LCD thermometer 2060 providing immediate temperature information to a user of treatment system 2000.

While not shown in FIG. 16, the temperature monitoring via temperature gauges operation described above permits the treatment system 2000 to maintain a treatment temperature (e.g., between 37° C. and 44° C., such as 42° C.) during a treatment session (e.g., during circulation or recirculation of a therapeutic solution). According to embodiments described above, when maintaining a treatment temperature, the control unit 70 (not shown in FIG. 16) monitors the temperature measurements of the temperature gauges 2010, 2020, 2030, 2040 and adjusts the operating temperature of the heater 1910 to operatively maintain a treatment temperature within the treatment system 2000 during a treatment session (e.g., during circulation or recirculation of a therapeutic solution). In an embodiment, the therapeutic solution may start the treatment session cold (e.g., at room or ambient temperature) at the beginning of a treatment session and then slowly be brought to a treatment temperature during the treatment session (e.g., during circulation or recirculation of therapeutic solution). According to an embodiment, the control unit 70 (not shown in FIG. 16) monitors the temperature measurements of the temperature gauges 2010, 2020, 2030, 2040 to determine when the therapeutic solution has been brought to a treatment temperature during the treatment session. Optionally, when the control unit 70 (not shown in FIG. 16) determines when the therapeutic solution has been brought to the treatment temperature, the control unit 70 disables (e.g., shuts off) the heater 1910.

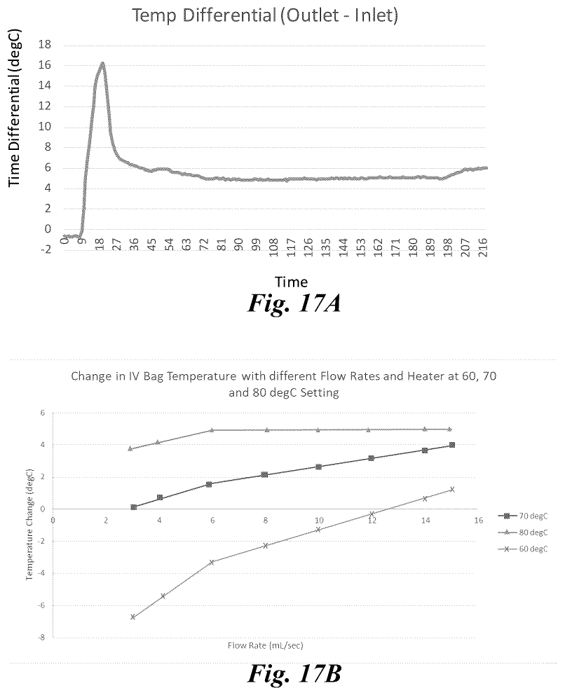

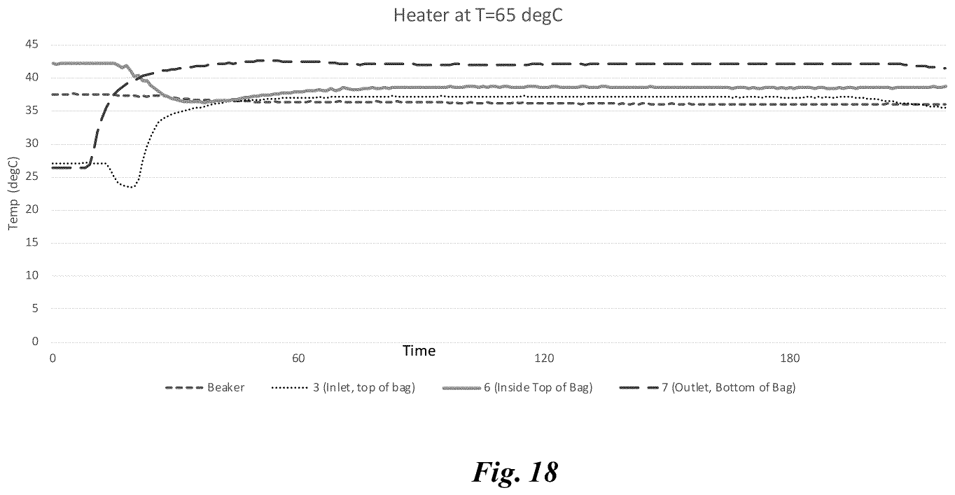

FIG. 17A is a plot of temperature differentials of a holding container as taken in the treatment system 2000 of FIG. 16 in accordance with an embodiment hereof. In particular, FIG. 17A plots the temperature differential of the treatment system 2000 between the temperature gauge 2020 and the temperature gauge 2040 as the heater 1910 (and the metal plate 1915) are heated to an operating temperature over time. As shown, the temperature differential spikes at the beginning of the treatment session, then stabilizes after approximately 72 seconds. FIG. 17B is a plot of temperature differentials of a holding container as taken in the treatment system 2000 of FIG. 16 with different heater settings and at different flow rates in accordance with an embodiment hereof. In particular, FIG. 17B plots three different settings of the heater 1910 (e.g., 60, 70, and 80 degC) and three different flow rates (e.g., 3 mL/sec, 6 mL/sec, 15 mL/sec) of a therapeutic fluid circulating through the closed fluid circuit of FIG. 16 describe above. FIG. 17B further shows the measured differences in temperature when treatment system 2000 is configured to maintain a 40 degC treatment temperature within holding container 1800 throughout a 20 minute period (e.g. a treatment session). While not shown in FIG. 17B, the ambient temperature within the simulated treatment chamber (e.g., a beaker)—as the therapeutic fluid was circulating through the chamber—was approximately 21 degC. As shown in FIG. 17B, treatment system 2000 achieved better heat exchange with a higher flow rate. Furthermore, depending on the flowrate, FIG. 17B showed that an operating temperature of the heater 1910 could be set to maintain the therapeutic fluid at a specific treatment temperature (e.g., 40 degC) during a treatment session (e.g., 20 minutes). FIG. 18 is a plot of fluid temperatures measured at different points in the treatment system 2000 of FIG. 16 in accordance with an embodiment hereof. In particular, FIG. 18 plots the temperature of a therapeutic fluid within the treatment system 2000 at the temperature gauges 2010, 2020, 2030, 2040 over time. According to FIG. 18, the temperature gauge 2010 is located in a simulated treatment chamber (e.g., a beaker), the temperature gauge 2020 is located near the inlet ports 1810 and 1820, the temperature gauge 2030 is located inside the lumen 1830, near the inlet ports 1810 and 1820, and the temperature gauge 2040 is located near the outlet port 1840. As shown, the measured temperatures fluctuate significantly at the beginning of the treatment session, then stabilize relative to each other after approximately 60 seconds.

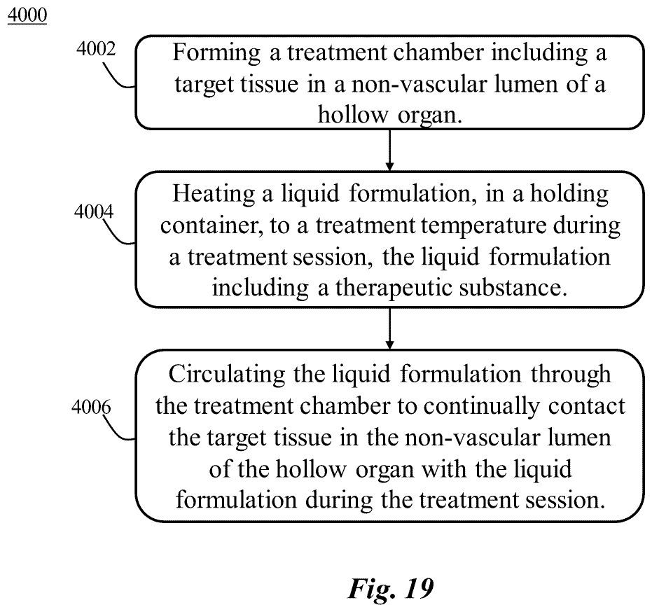

FIG. 19 provides a flow diagram that illustrates a method 4000 for delivery of a liquid formulation having a therapeutic substance to a target tissue of a hollow organ in accordance with an embodiment herein. According to FIG. 19, the method 4000 is provided for administering a therapeutic substance to a target tissue within a hollow organ in a subject. Method 4000 includes a step 4002 for forming a treatment chamber (e.g., treatment chamber 35, shown in FIG. 1) including the target tissue (e.g., polyps 21, shown in FIG. 1) in a non-vascular lumen of the hollow organ (e.g., colon 20, shown in FIG. 1). As shown in FIG. 19, a step 4004 includes heating (e.g., using heater 1910, shown in FIG. 15) a liquid formulation (e.g., liquid drug solution 30, shown in FIG. 1), in a holding container (e.g., holding container 1800, shown in FIG. 14), to a treatment temperature during a treatment session, the liquid formulation including the therapeutic substance. The method 4000 further includes circulating the liquid formulation (e.g., liquid drug solution 30) through the treatment chamber (e.g., treatment chamber 35) to continually contact the target tissue (e.g., polyps 21) in the non-vascular lumen of the hollow organ (e.g., colon 20) with the liquid formulation during the treatment session, as noted in a step 4006.

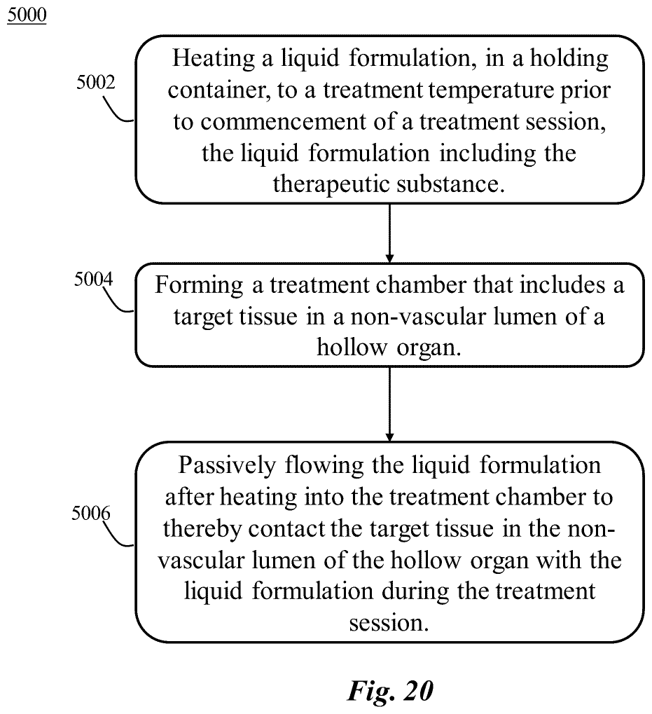

FIG. 20 provides a flow diagram that illustrates a method 5000 for delivery of a therapeutic solution to a target tissue area of an internal body organ, according to an embodiment herein. According to FIG. 20, the method 5000 is provided for administering a therapeutic substance to a target tissue within a hollow organ in a subject. The method 5000 includes a step 5002 of heating (e.g., using a heater 1910, shown in FIG. 15) a liquid formulation (e.g., liquid drug solution 30, shown in FIG. 1), in a holding container (e.g., holding container 1800), to a treatment temperature prior to commencement of a treatment session, the liquid formulation (e.g., liquid drug solution 30) including a therapeutic substance. As shown, a step 5004 provides for forming a treatment chamber (e.g., treatment chamber 35, shown in FIG. 1) that includes the target tissue (e.g., polyps 21, shown in FIG. 1) in a non-vascular lumen of the hollow organ (e.g., colon 20, shown in FIG. 1). Method 5000 further provides for passively flowing the liquid formulation (e.g., liquid drug solution 30) after heating into the treatment chamber (e.g., treatment chamber 35) to thereby contact the target tissue (e.g., polyps 21) in the non-vascular lumen of the hollow organ (e.g., colon 20) with the liquid formulation (e.g., liquid drug solution 30) during the treatment session, as a step 5006.

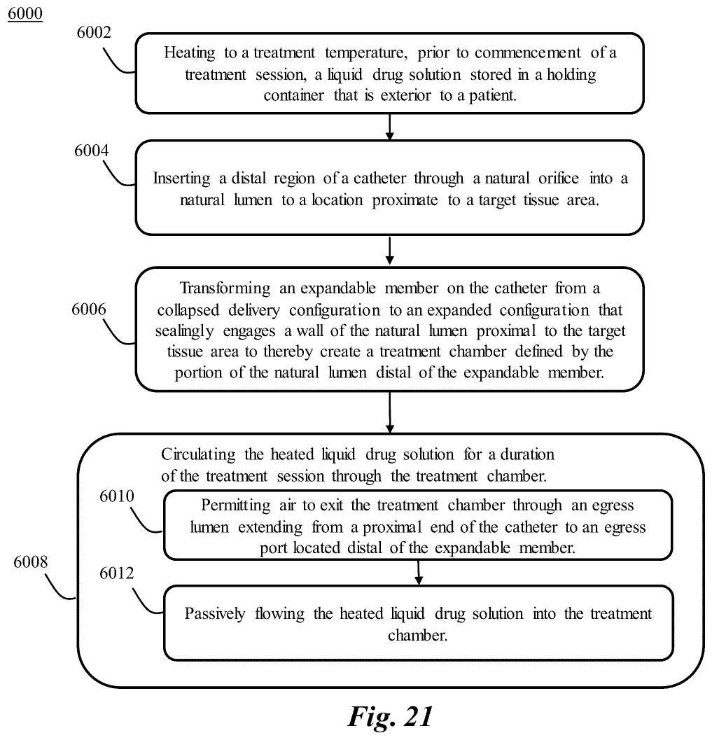

FIG. 21 provides a flow diagram that illustrates a method 6000 for delivery of a therapeutic solution to a target tissue area of an internal body organ in accordance with another embodiment herein. According to FIG. 21, the method 6000 is provided for local delivery of a heated liquid drug solution to a target tissue area surrounding a natural lumen extending through an internal body organ of a patient. The method 6000 includes a step 6002 of heating to a treatment temperature (e.g., using heater 1910, shown in FIG. 15), prior to commencement of a treatment session, a liquid drug solution (e.g., liquid drug solution 30, shown in FIG. 1) stored in a holding container (e.g., holding container 1800, shown in FIG. 14) that is positioned exterior to the patient. A step 6004 of the method 6000 includes inserting a distal region of a catheter (e.g. a catheter 810, shown in FIG. 8) through a natural orifice (e.g., patient's mouth, shown in FIG. 8) into a natural lumen (e.g., bronchus 820, shown in FIG. 1) to a location proximate to a target tissue area (e.g., walls bronchus 820, shown in FIG. 8). A step 6006 of the method 6000 includes transforming an expandable member (e.g., expandable member 811, shown in FIG. 8) on the catheter (e.g., catheter 810) from a collapsed delivery configuration (e.g., expandable members 11a, shown in FIG. 2) to an expanded configuration (e.g., expandable members 11b, 11c, and 11d, shown in FIG. 2) that sealingly engages a wall of the natural lumen (e.g., bronchus 820) proximal to the target tissue area (e.g., walls of bronchus 820) to thereby create a treatment chamber (e.g., treatment chamber 35, shown in FIG. 1) defined by a portion of the natural lumen (e.g., bronchus 820) distal of the expandable member (e.g., expandable member 811).

The method 6000 further includes circulating the heated liquid drug solution (e.g., liquid drug solution 30, shown in FIG. 1) for the duration of a treatment session through the treatment chamber (e.g., treatment chamber 35, shown in FIG. 1) as noted in a step 6008. The step 6008 includes a step 6010 for permitting air to exit the treatment chamber (e.g., treatment chamber 35) through an egress lumen extending from a proximal end of the catheter (e.g., catheter 810) to an egress port (e.g., egress port 814, shown in FIG. 8) located distal of the expandable member (e.g., expandable member 811, shown in FIG. 8). The step 6008 of the method 6000 further includes passively flowing the heated liquid drug solution (e.g., liquid drug solution 30) into the treatment chamber (e.g., treatment chamber 35) as a step 6012.

### Endobronchial Lavage

In accordance with the present disclosure, an endobronchial lavage (EBL) procedure is performed on the lungs of a subject or patient using a catheter system that utilizes a combination of balloon catheters to isolate each lung at the level of a main bronchus. In the catheter system, a first catheter is used for ventilation of a first lung while a second catheter, which may be referred to herein as a lavage delivery catheter, is used to form a treatment chamber in a second lung that is to be treated with a drug (therapeutic substance) solution by lavage, and a third catheter is positioned within a trachea to provide ventilation while the first and second catheters are being placed within the lungs. The lavage delivery catheter has multiple lumens to allow the lavage drug solution (also referred to herein as a liquid lavage solution), contained in a bag or other containment vessel, to enter the second lung while simultaneously allowing air to escape from the second lung. In a method hereof, a lung to be treated is filled to completion with the lavage drug solution until no residual air is left within the lung being treated. The lavage drug solution is circulated and allowed to be in the treatment chamber of the lung for a therapeutic period of time, for instance several minutes, to provide sufficient exposure for the drug to fully integrate into spaces between the lung tissues. In an embodiment, as the lavage solution enters and circulates through the treatment chamber of the lung, the air previously in that lung is contained, captured in a bag or otherwise filtered, preventing chemical or biologic exposure of the clinician and any staff during the EBL procedure. Finally, the lavage drug solution is removed from the treated lung into the original bag or other containment vessel, and air is reintroduced to the treated lung during the removal process. The EBL procedure may then be conducted on the other lung if desired. In an embodiment, the total length of the procedure may be less than 10 to 20 minutes per lung.

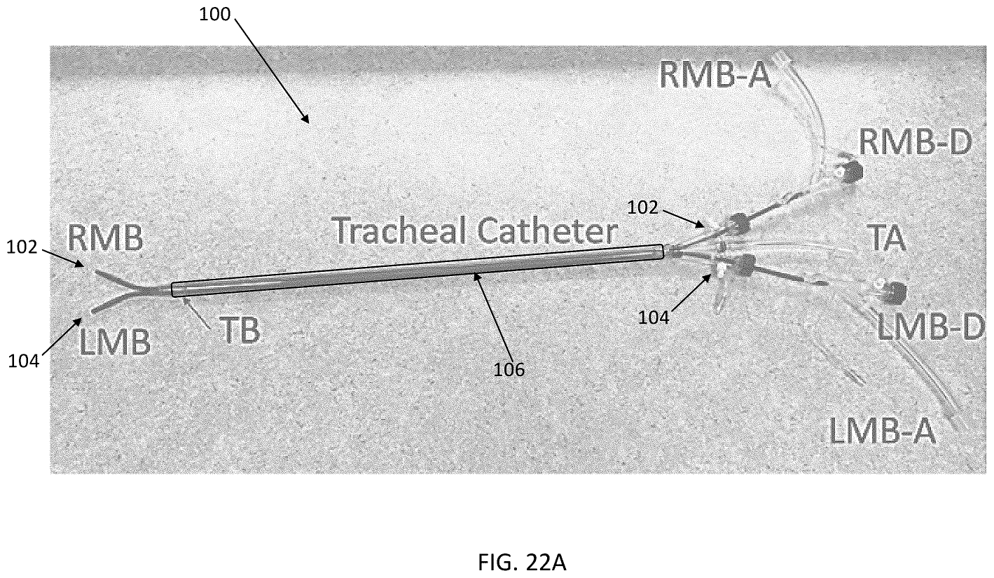

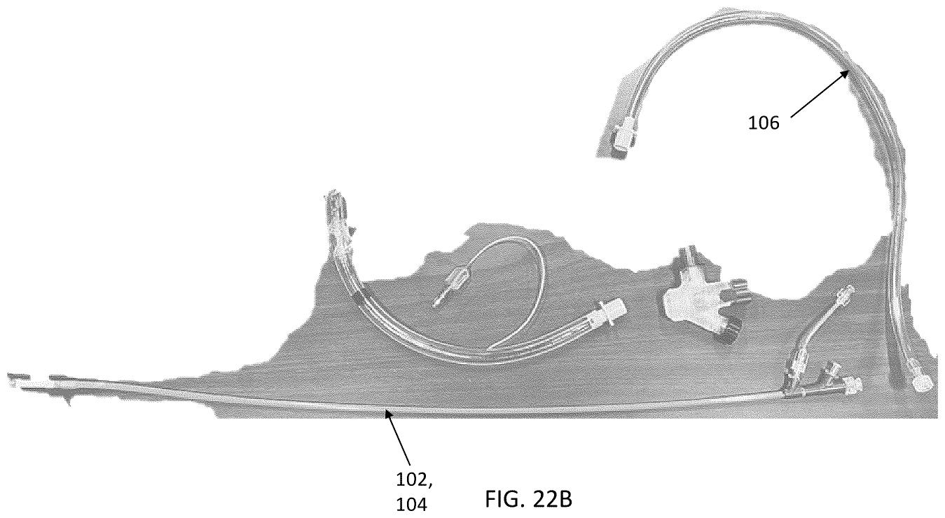

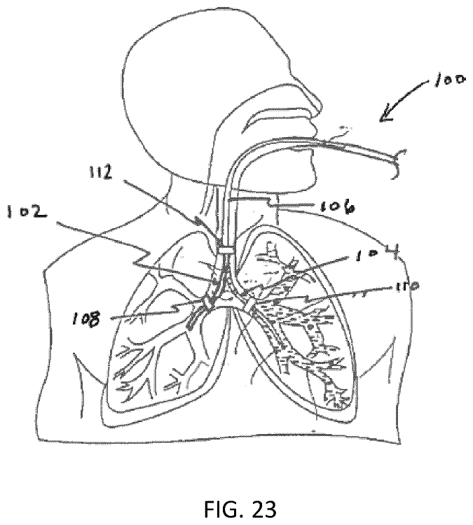

FIGS. 22A, 22B, and 23 illustrate a catheter system 100 configured in accordance with an embodiment of the present technology. FIG. 22A illustrates the catheter system 100 in an assembled state. FIG. 22B illustrates the individual components of catheter system 100 in a disassembled state. FIG. 23 illustrates the catheter system 100 during use. The catheter system 100 includes three catheter components, with first and second catheters 102, 104 being configured to be placed into the bronchus and a third catheter 106 being configured to be placed in the trachea and through which the other two catheters slidingly extend. The nested first and second catheters 102, 104 are used to occlude the left main bronchus (LMB) or right main bronchus (RMB) individually with dedicated balloons 108, 110 located along distal portions thereof proximal of their respective distal ends. The first and second catheters 102, 104 are nested and slidable within the third catheter 106 that has a balloon 112 on a distal portion thereof that is sized for expansion within the trachea to occlude the trachea for temporary ventilation during placement of the first and second catheters 102, 104. In addition, the third catheter 106 is configured to be sufficiently stable as to provide support for the first and second catheters 102, 104 during an endobronchial lavage procedure. In an embodiment, all three catheters have ventilator connectors such that each of the first, second and/or third catheters catheter 102, 104, 106 may be connected to a ventilator at any point during an interventional procedure for ventilation support.

Each of the catheters 102, 104, 106 has an elongated flexible shaft made of a biocompatible material for deployment within a target region of a patient or subject's respiratory system, which in this example is the trachea and lungs as shown in FIG. 2. A balloon 108, 110, 112 is mounted about a distal region of each of the respective catheters 102, 104, 106 such that when expanded each balloon comes into sealing contact with the inner wall of the trachea or bronchus of the lung as a specific application dictates. A balloon for use in the present technology may be a mechanically operated scaling element or other expandable member that is inflatable with a fluid that may be either a gas or a liquid. One or both of the first and second catheters 102, 104 includes a pair of ports distal of its respective balloon that fluidly communicate a treatment area of the lungs with respective lumens (not shown) of the catheter, wherein at least two such lumens extend proximally through the first and second catheters 102, 104 to terminate at respective connecting ports 114, 116, 118, 120 located at the proximal ends thereof, as shown in FIG. 22A.

More particularly, each of the first and second catheters 102, 104 for use within the lungs has a drug delivery port 114, 116, respectively, that can be used for drug delivery via its associated lumen and distal exit port (not shown). In an EBL procedure in accordance with the present disclosure and as shown in FIG. 23, the first catheter 102 is used to ventilate one lung while the second catheter 104 is used for drug delivery in the other lung to be treated. In an embodiment, at least the second catheter 104 used for drug delivery in the lung to be treated has at least two lumens that extend therethrough to allow the lavage drug solution to enter the lung via a first lumen while simultaneously allowing air to escape from the lung via a second lumen in order to avoid creating elevated lung pressures that could damage the alveoli.

In an embodiment, the catheter system 100 may include an additional catheter with a camera at a distal tip thereof that is sized to fit within any of the prior three catheters 102, 104, 106 to provide direct visualization during catheter placement and drug delivery.

In an embodiment, a liquid lavage drug solution may optionally be heated, and admitted or pumped into the lung to be treated via an ingress port, such as one of ports 116, 120 of the second catheter 104. As the liquid lavage drug solution fills the lung, preferably from bottom to top, air is purged from the lung via an egress port and dedicated lumen of the catheter (not shown). In this way, the lung is filled with the liquid lavage drug solution. a liquid drug solution may be heated to a treatment temperature prior to delivery to the lung, and the liquid drug solution may be maintained at the treatment temperature during a treatment session as it is circulated throughout the lung.

The catheter system 100 described above may be used in a method for performing an endobronchial lavage (EBL) procedure as a medical countermeasure against inhaled chemical agents, including lung injury associated with phosgene gas exposure. Additionally, the catheter system 100 may be used to mitigate and treat lung damage from other pulmonary chemical agents. An endobronchial lavage is a method of directly delivering therapeutics to the interior of the lungs, and during an EBL procedure, the entire lung of a subject or patient under anesthesia may be briefly filled with a solution containing appropriate drugs or medications to address injury resulting from an inhaled chemical agent. In an embodiment, a lavage solution including sodium bicarbonate and albuterol sulfate will be administered in a therapeutically suitable dose that is sufficient to clear debris from the lung, reduce the severity of (or cure/eliminate) the injury, and accelerate recovery while maintaining safety. In an embodiment, a therapeutically suitable dose of sodium bicarbonate and albuterol sulfate contained within a lavage solution may mitigate pulmonary edema (excess fluid in the lungs) and other acute lung injuries that could occur during a chemical attack with phosgene gas. In an embodiment, a therapeutically suitable dose of sodium bicarbonate and albuterol sulfate contained within a lavage solution may be used in an EBL procedure hereof as an effective medical countermeasure against other pulmonary chemical threats, such as chlorine, sulfur mustard, opioids, nerve agents, and/or any other inhaled chemical threat. Additionally, or alternatively, the catheter system 100 may be used to treat other lung damage, such as due to disease.

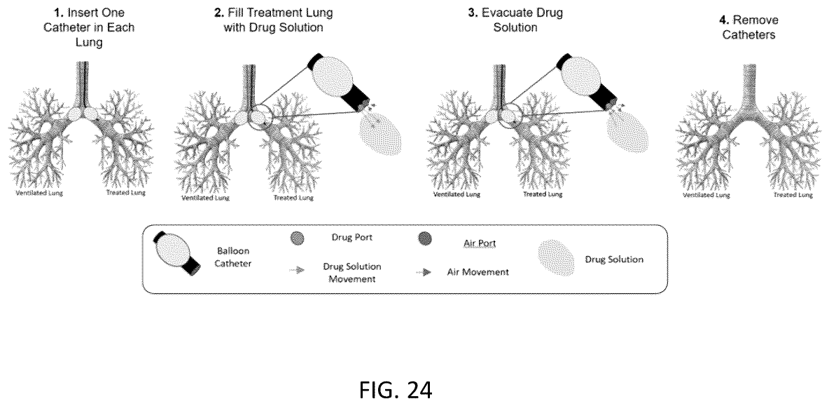

With reference to FIG. 24, an EBL procedure in accordance with the disclosure is depicted. An initial step of the EBL procedure is to advance a tracheal catheter 106 with the nested first and second catheters 102, 104 and place each of the bronchial first and second catheters 102, 104 at the level of the main bronchus in each lung. During this step, the tracheal catheter balloon 112 is inflated within the trachea and the tracheal catheter 106 is used to provide ventilation support as the bronchial first and second catheters 102, 104 are individually positioned. Providing such ventilation by the tracheal catheter 106 permits time from the initial advancement of the catheter system into the subject to ventilation to be less than one minute. The next step of the EBL procedure transitions ventilation from the tracheal catheter 106 to one of the bronchial first and second catheters 102, 104, which in FIG. 23 is the first catheter 102. The other bronchial catheter 102, 104 can then be used to create a treatment chamber within the lung, distal of a balloon thereof, for receiving the liquid lavage solution containing a therapeutic substance/drug, which in FIG. 23 is the second catheter 104 and its balloon 110. In accordance with the disclosure, a benefit of using a two bronchial catheters with a tracheal catheter instead of a double lumen endotracheal tube for this procedure provides added safety and redundancy to protect the ventilated lung should the drug delivery balloon fail. Thereby the catheter system 100 as disclosed herein ensures ventilation at all times.

With continued reference to FIG. 24, the treatment chamber of the lung is filled to completion with the lavage solution, which may be heated, until no residual air is left within the lung being treated. The lavage solution is then circulated and allowed to be in the treatment chamber of the lung for a therapeutic period of time, such as several minutes (e.g., about 10 to 20 minutes), for the drug to fully integrate into the lung tissue. Finally, the lavage solution is removed from the lung into a dedicated bag or other container, and air is reintroduced to the lung. The EBL procedure can then be conducted on the other lung if desired. In an embodiment, the total length of the EBL procedure is less than 20 minutes per lung.

In an embodiment hereof, a catheter system for EBL drug delivery may include sterile packaged disposables (catheters, and drug/air bag,), capital equipment (drug pump, circulation pump, optional heating element, image processor, and imaging screen). For instance, a heater may be used to increase the temperature of the drug solution before a lavage procedure and during drug delivery, while a drug pump may be used to pull the drug lavage solution out of the lungs back into a circulation bag or other container so it can be re-heated and delivered back into the lungs to maintain temperature. In an embodiment, a pressure relief circuit that provides a fluid relief path to avoid high fluid pressures, both of which could be damaging to lung tissue, may be utilized in an EBL procedure in accordance herewith. In some implementations, liquid may be supplied to the lung via a gravity feed, (e.g., without a pump). Additionally, the liquid may be naturally expelled by the lung, e.g., natural contraction of the lungs.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described exemplary embodiments but should be defined only in accordance with the appended claims and their equivalents. It will also be apparent that all hollow organs are eligible for both single and multiple balloon configurations of the devices, systems and methods described herein. It will also be understood that each feature of each embodiment discussed herein, and of each reference cited herein, can be used in combination with the features of any other embodiment.

Reference may be made to the spatial relationships between various components and to the spatial orientation of various aspects of components as depicted in the attached drawings. However, as will be recognized by those skilled in the art after a complete reading of the present disclosure, the devices, components, members, apparatuses, etc. described herein may be positioned in any desired orientation. Thus, the use of terms such as “above,” “below,” “upper,” “lower,” “top,” “bottom,” or other similar terms to describe a spatial relationship between various components or to describe the spatial orientation of aspects of such components, should be understood to describe a relative relationship between the components or a spatial orientation of aspects of such components, respectively, as the components described herein may be oriented in any desired direction. When used to describe a range of dimensions and/or other characteristics (e.g., time, pressure, temperature, distance, etc.) of an element, operations, conditions, etc., the phrase “between X and Y” represents a range that includes X and Y.

For example, it is to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points of reference and do not limit the present invention to any particular orientation or configuration. Further, the term “exemplary” is used herein to describe an example or illustration. Any embodiment described herein as exemplary is not to be construed as a preferred or advantageous embodiment, but rather as one example or illustration of a possible embodiment.

Further, the present disclosure may repeat reference numerals and/or letters in the various examples. This repetition is for the purpose of simplicity and clarity and does not in itself dictate a relationship between the various embodiments and/or configurations discussed.

Similarly, when used herein, the term “comprises” and its derivations (such as “comprising,” etc.) should not be understood in an excluding sense, that is, these terms should not be interpreted as excluding the possibility that what is described and defined may include further elements, steps, etc. Meanwhile, when used herein, the term “approximately” and terms of its family (such as “approximate,” etc.) should be understood as indicating values very near to those which accompany the aforementioned term. That is to say, a deviation within 10% or other reasonable limits from an exact value should be accepted, because a skilled person in the art will understand that such a deviation from the values indicated is inevitable due to measurement inaccuracies, etc. The same applies to the terms “about” and “around” and “substantially”.

As used herein, unless expressly stated to the contrary, use of the phrase “at least one of,” “one or more of,” “and/or,” variations thereof, or the like are open-ended expressions that are both conjunctive and disjunctive in operation for any and all possible combination of the associated listed items. For example, each of the expressions “at least one of X, Y and Z,” “at least one of X, Y or Z,” “one or more of X, Y and Z,” “one or more of X, Y or Z” and “X, Y and/or Z” can mean any of the following: 1) X, but not Y and not Z; 2) Y, but not X and not Z; 3) Z, but not X and not Y; 4) X and Y, but not Z; 5) X and Z, but not Y; 6) Y and Z, but not X; or 7) X, Y, and Z.

Additionally, unless expressly stated to the contrary, the terms “first,” “second,” “third,” etc., are intended to distinguish the particular nouns they modify (e.g., element, condition, node, outlet, inlet, valve, module, activity, operation, etc.). Unless expressly stated to the contrary, the use of these terms is not intended to indicate any type of order, rank, importance, temporal sequence, or hierarchy of the modified noun. For example, “first X” and “second X” are intended to designate two “X” elements that are not necessarily limited by any order, rank, importance, temporal sequence, or hierarchy of the two elements. Further as referred to herein, “at least one of” and “one or more of” can be represented using the “(s)” nomenclature (e.g., one or more element(s)).

1. A method for administering a therapeutic substance to a target tissue within a hollow organ in a subject, comprising:
forming a treatment chamber comprising the target tissue in a non-vascular lumen of the hollow organ; and
circulating a liquid formulation through the treatment chamber to continually contact the target tissue in the non-vascular lumen of the hollow organ with the liquid formulation during a treatment session, the liquid formulation comprising a therapeutic substance.

2.-20. (canceled)

21. A method for administering a therapeutic substance to a target tissue within a hollow organ in a subject, comprising:
forming a treatment chamber comprising the target tissue in a non-vascular lumen of the hollow organ; and
passively flowing a liquid formulation into the treatment chamber to thereby contact the target tissue in the non-vascular lumen of the hollow organ with the liquid formulation during a treatment session, the liquid formulation comprising the therapeutic substance.

22. The method of claim 21, wherein a treatment temperature is between 37° C. and 44° C.

23. (canceled)

24. The method of claim 21, further comprising circulating the liquid formulation through the treatment chamber to continually contact the target tissue in the non-vascular lumen of the hollow organ with the liquid formulation during the treatment session.

25.-31. (canceled)

32. The method of claim 24, further comprising holding in a holding container for a holding period prior to commencement of the treatment session, wherein the holding period is approximately 10 minutes.

33. (canceled)

34. The method of claim 21, wherein the treatment chamber is formed between a proximal balloon and a distal balloon of a catheter.

35. The method of claim 21, wherein the treatment chamber is formed distal of a balloon of a catheter.

36. The method of claim 21, wherein the hollow organ is at least a portion of one of an esophagus, a stomach, a colon, a respiratory tract, a lung, a female genital tract, a uterus and a urinary tract.

37. The method of claim 21, wherein the therapeutic substance is one or more of diclofenac, sodium bicarbonate, dexamethasone sodium phosphate, or combinations thereof.

38. The method of claim 21, wherein the hollow organ is a lung and the therapeutic substance comprises an anti-inflammatory agent.

39. The method of claim 21, wherein the step of passively flowing the liquid formulation after heating into the treatment chamber comprises gravity feeding the liquid formulation into the treatment chamber.

40. A method for treating a cancer target tissue within a hollow organ of a subject, comprising:
heating a liquid formulation, in a holding container, to a treatment temperature prior to commencement of a treatment session, the liquid formulation comprising a therapeutic drug;
forming a treatment chamber comprising the cancer target tissue in a non-vascular lumen of the hollow organ; and
passively flowing the liquid formulation after heating into the treatment chamber to thereby contact the cancer target tissue in the non-vascular lumen of the hollow organ with the liquid formulation during the treatment session.

41.-53. (canceled)

54. A method for local delivery of a heated liquid drug solution to a target tissue area surrounding a natural lumen extending through an internal body organ of a patient, the method comprising:
heating, prior to commencement of a treatment session, a liquid drug solution stored in a holding container, exterior to the patient, to a treatment temperature;
inserting a distal region of an elongate flexible catheter through a natural orifice into the natural lumen to a location proximate to the target tissue area;
transforming an expandable member on the catheter from a collapsed delivery configuration to an expanded configuration that sealingly engages a wall of the natural lumen proximal to the target tissue area to thereby create a treatment chamber defined by a portion of the natural lumen distal of the expandable member; and
circulating the heated liquid drug solution for the duration of the treatment session through the treatment chamber, wherein the step of circulating the heated liquid drug solution comprises
permitting air to exit the treatment chamber through an egress lumen extending from a proximal end of the catheter to an egress port located distal of the expandable member, and
passively flowing the heated liquid drug solution into the treatment chamber.

55.-61. (canceled)

62. An apparatus for local delivery of a liquid drug solution to a target tissue area of an internal body organ of a patient, the apparatus comprising:
a catheter having
an elongate flexible shaft,
an expandable member disposed about a distal region of the elongate flexible shaft and being transformable between a collapsed delivery configuration and an expanded configuration for sealing against a wall of a non-vascular natural lumen extending through the target tissue area to form a sealed treatment chamber defined by the wall of the non-vascular natural lumen distal of the expandable member,
an air egress lumen extending from a proximal end of the elongate flexible shaft to an air egress port disposed in the distal region of the elongate flexible shaft, wherein the air egress port is located at a distal end of the expandable member, and is immediately adjacent to the expandable member, and
a liquid flow lumen for delivery of the liquid drug solution into the sealed treatment chamber, and extending from the proximal end of the elongate flexible shaft to a liquid flow port disposed in the distal region of the elongate flexible shaft, at a location distal to the air egress port;
a holding container configured to hold the liquid drug solution, the holding container being fluidly coupled with the liquid flow lumen of the catheter external to the patient; and
a heater configured to heat the liquid drug solution held within the holding container,
wherein the air egress port is configured to facilitate air evacuation when the heated liquid drug solution is being delivered into the sealed treatment chamber,
wherein the heated liquid drug solution is heated to a treatment temperature prior to delivery of the heated liquid drug solution into the sealed treatment chamber,
wherein, after the sealed treatment chamber is filled with the heated liquid drug solution, the air egress lumen, holding container and the liquid flow lumen are configured to be fluidly coupled with each other and the sealed treatment chamber to form a closed fluid circuit, and
wherein the closed fluid circuit is configured to permit the heated liquid drug solution to circulate through the sealed treatment chamber.

63.-71. (canceled)

72. A method for administering a therapeutic substance to a target tissue within a lung in a subject for treating a lung injury due to a chemical or smoke exposure, comprising:
advancing a catheter system into a trachea of the subject to place a tracheal catheter of the catheter system within the trachea such that an expanded balloon of the tracheal catheter contacts and seals against a distal portion of the trachea;
positioning a first catheter of the catheter system, which slidingly extends through the tracheal catheter, within a first lung of the subject such that an expanded balloon of the first catheter contacts and seals against a main bronchus of the first lung;
positioning a second catheter of the catheter system, which slidingly extends through the tracheal catheter, within a second lung of the subject such that an expanded balloon of the second catheter contacts and seals against a main bronchus of the second lung;
forming a treatment chamber comprising the target tissue in one of the first and second lungs, wherein the treatment chamber is formed distal of the expanded balloon of one of the first and second catheters;
conducting an endobronchial lavage (EBL) procedure in the lung to be treated, wherein the EBL procedure includes delivering a liquid lavage solution into the treatment chamber in the lung to be treated to continually contact the target tissue with the liquid lavage solution during a treatment session.

73. The method of claim 72, wherein the therapeutic substance is a therapeutically suitable dose of one or more of the following drugs, sodium bicarbonate, dexamethasone sodium phosphate, diclofenac, and albuterol sulfate contained within the liquid lavage solution.

74. The method of claim 73, wherein following conducting the endobronchial lavage (EBL) procedure in a lung to be treated, pulmonary edema (excess fluid) and/or other lung injuries caused by the chemical exposure with a chemical agent, for e.g., phosgene gas, are reduced or eliminated or smoke inhalation.
